(19) United States
(12) Patent Application Publication
(10) Pub. No.: US 2024/0115606 A1
(43) Pub. Date: Apr. 11, 2024

(54) Cell-Based Therapeutics Targeting CD70

(71) Applicant: Universiteit Antwerpen, Antwerpen (BE)

(72) Inventors: Astrid VAN DEN EYNDE, Borgerhout (BE); Evelien SMITS, Antwerpen (BE); Patrick PAUWELS, Retie (BE); Julie JACOBS, Weelde (BE)

(21) Appl. No.: 18/267,231

(22) PCT Filed: Dec. 15, 2021
(86) PCT No.: PCT/EP2021/085976

§ 371 Date: Jun. 14, 2023

(30) Foreign Application Priority Data
Dec. 15, 2020 (EP) 20214352.5

(51) Int. Cl. A61K 35/17; A61K 39/00; A61P 35/00; C07K 14/54; C07K 16/28; C12N 5/0783

(52) U.S. Cl. CPC A61K 35/17; A61K 39/4613; A61K 39/4631; A61K 39/464429; A61K 39/46444; A61P 35/00; C07K 14/5434; C07K 14/5443; C07K 16/2875; C12N 5/0646; A61K 2239/13; A61K 2239/22; A61K 2239/50

(57) ABSTRACT

The invention provides a natural killer (NK) cell engineered to express a chimeric antigen receptor (CAR), wherein the CAR comprises the extracellular domain of CD27 or a CD70-binding portion thereof, as well as compositions comprising the engineered NK cell, methods for producing the engineered NK cell, and therapeutic applications of the engineered NK cell, such as for treating neoplastic diseases.

### FIELD

The invention is broadly applicable in the medical field and more specifically concerns immune cell-based therapeutics useful particularly in methods of treating neoplastic diseases.

### BACKGROUND

CD70 (Cluster of Differentiation 70) protein, a tumor necrosis factor (TNF)-related molecule, also denoted as CD27 ligand (CD27L), is under normal circumstances only transiently expressed on activated T and B cells and mature dendritic cells. However, constitutive expression of CD70 has been described on malignant cells in a range of solid and haematological malignancies. Through its receptor, CD27, CD70 expression on malignant cells can facilitate evasion of the immune system by inter alfa induction of T cell apoptosis, T cell exhaustion and increasing the amount of suppressive regulatory T cells (Tregs). In view of CD70 being expressed on highly activated lymphocytes, such as in T- and B-cell lymphomas, contrasted with short transient CD70 expression on healthy lymphocytes, anti-CD70 antibodies have been proposed as a potential treatment for CD70 positive malignancies.

More recently, the present inventors have characterised the expression of CD70 on cancer associated fibroblasts (CAFs) (Jacobs et al. Oncoimmunology. 2018, vol. 7(7), e1440167). CAFs are a dominant component of the tumor microenvironment (TME) and have been proposed to play a role in the proliferative and invasive behaviour of the tumor as well as in immune evasion. The authors reported that the number of CD70 positive CAFs increased with stage (T1-T4) of colorectal cancer (CRC) specimens (see FIG. 2 herein) and that CD70 expression acted as a significant negative predictor for overall survival and progression-free survival in CRC patients.

WO 2016/093878 concerns anti-CD70 chimeric antigen receptors (CARs). WO 2019/213610 concerns natural killer (NK) cells engineered to express CARs with immune checkpoint blockade.

### SUMMARY

The present invention is at least in part based on the inventors' innovative insights and experimental evaluation focusing on the feasibility and therapeutic usefulness of targeting CD70 positive cancer cells and/or CD70 positive tumor microenvironment cells such as cancer associated fibroblasts (CAFs) using natural killer (NK) cells engineered to express a chimeric antigen receptor (CAR) which comprises the extracellular domain of CD27 or a CD70-binding portion thereof.

In an aspect the invention provides a natural killer (NK) cell engineered to express a chimeric antigen receptor (CAR), wherein the CAR comprises the extracellular domain of CD27 or a CD70-binding portion thereof.

A further aspect provides a pharmaceutical composition comprising the engineered NK cell and a pharmaceutically acceptable carrier.

Another aspect provides a method for producing the engineered NK cell comprising introducing a nucleic acid encoding the CAR in an expressible format into a starting population of NK cells. Optionally and advantageously, the NK cells which then comprise said nucleic acid and which are capable of expressing the CAR may be selected and/or expanded.

An aspect provides the engineered NK cell or the pharmaceutical composition for use in therapy. A further aspect provides the engineered NK cells or the pharmaceutical composition for use in a method of treating a neoplastic disease. A related aspect provides a method for treating a subject in need thereof, in particular a subject having a neoplastic disease, the method comprising administering to the subject a therapeutically effective amount of the engineered NK cells or the pharmaceutical composition.

The present invention thus pertains to an improved immune cell-based therapeutic agent employing a specifically designed chimeric antigen receptor (CAR) having antigenic specificity for CD70, with ability to effectively eliminate CD70 positive cells, which may include cancerous and/or tumor microenvironment (TME) cells such as CAFs. Whether or not a tumor contains CD70 positive cancerous cells, eradication of CD70 positive TME cells can be of considerable therapeutic benefit, since the latter cells, such as in particular CD70 positive CAFs, can be largely responsible for the immunosuppressive character of the tumor microenvironment, and their eradication can thus make the tumor more susceptible to clearance such as by the action of the patient's own immune system or by other (immuno-)therapies.

These and further aspects and preferred embodiments of the invention are described in the following sections and in the appended claims. The subject-matter of the appended claims is hereby specifically incorporated in this specification.

### BRIEF DESCRIPTION OF DRAWINGS

FIG. 1 illustrates the molecular mechanisms believed to underlie or contribute to the therapeutic effects elicited by the immune cell-based agents according to certain embodiments of the present invention. A natural killer cell engineered to express a chimeric antigen receptor (CAR) (1), wherein the CAR comprises the extracellular domain of CD27 or a CD70-binding portion thereof (2), specifically binds to a CD70 molecule (3) exposed on the surface of a cancer associated fibroblast (CAF) (4) (upper panel) or a cancerous cell (5) (lower panel) in a tumor, thereby driving or facilitating the elimination of these CD70 positive cells and of the tumor.

FIG. 2 reproduces a graph reported by Jacobs et al. (supra) quantifying the expression of CD70 on fibroblasts (expressed as % CD70 positive fibroblasts, y axis) in normal (N), adenoma (A), in situ carcinoma (Tis) and T1-T4 colorectal cancer (CRC) specimens, graded as − (<1%), + (1-10%), ++ (11-50%), +++ (>50% of CAFs express CD70).

FIG. 3 (A, B) show overlay of representative histograms of CD27 expression on NK-92 cells 24 hours after electroporation without CAR mRNA (MOCK), with CAR mRNA containing an IL-15 cytokine cassette (CD27-CAR) and with CAR mRNA without an IL-15 cytokine cassette (CD27-CAR w/o IL-15). With further optimisation and improvement of the electroporation protocol, the histograms are even more to the right, showing increased expression of the CAR (B).

FIG. 4 shows characterization of the CD70-targeting CAR-NK-92 cells. (A-B) Graphs showing the percentage overtone (A) and delta mean fluorescence intensity (LMFI) (B) of CD27 expression on NK-92 cells 24 hours after electroporation without CAR mRNA (MOCK), with CAR mRNA without an IL-15 cytokine cassette (CD27-CAR) and with an IL-15 cytokine cassette (CD27-CAR with IL-15). (C) concentration IL-15 produced in the supernatants of NK-92 cells after electroporation with MOCK, CD27-CAR and CD27-CAR with IL-15. Dotted line represents the detection limit of the assay. Experiments were performed at least in triplicate. Error bars represent standard deviation. * P≤0.05, ΔMFI=specific CD27 mAb staining−isotype control.

FIG. 5 shows in vitro cytotoxic potential of CD70-targeting CAR-NK-92s towards CD70+ target cell lines. Graphs showing the percentage cell death of the different CD70⁺ target cell lines (Raji, PANC-1, LIM2099, and RLT-PSC) after 4 hours of co-culture with NK-92 cells electroporated without CAR mRNA (MOCK), with CAR mRNA without an IL-15 cytokine cassette (CD27-CAR) and with an IL-15 cytokine cassette (CD27-CAR with IL-15), 24 hours after electroporation. The percentage dead target cells are identified as Annexin V⁺ and/or 7-AAD⁺ cells with flow cytometry. Experiments were performed at least in triplicate. Error bars represent standard deviation. * P≤0.05.

FIG. 6 shows blocking of the antigen-recognition domain of the CAR, which demonstrates CAR-specific killing. Graphs show the percentage viable cells of the CD70⁺ Raji target cell line alone (Raji baseline) or after 4 hours of coculture with the NK-92 cells electroporated without CAR mRNA (MOCK), with CAR mRNA without an IL-15 cytokine cassette (CD27-CAR) and with an IL-15 cytokine cassette (CD27-CAR with IL-15), 24 hours after electroporation. The effector cells were incubated overnight and during the coculture with different concentrations (10 μg/mL, 50 μg/mL, or 100 μg/mL) of a neutralizing anti-CD27 monoclonal antibody or IgG1 isotype control. The percentage viable target cells are identified as Annexin V⁻ and 7-AAD⁻ cells with flow cytometry. Experiments were performed at least in triplicate. Errors bars represent standard deviation. * P≤0.05.

FIG. 7 shows stimulation of the CD70-targeting CAR-NK-92s with IL-15 or IL-21 improves cytotoxic potential. Graphs show the percentage viable cells of the different CD70⁺ target cell lines (Raji, PANC-1, LIM2099 and RLT-PSC) after 4 hours of coculture with the NK-92 cells electroporated without CAR mRNA (MOCK), with CAR mRNA without an IL-15 cytokine cassette (CD27-CAR) and with an IL-15 cytokine cassette (CD27-CAR with IL-15), 24 hours after electroporation. The CD27-CAR NK-92 cells were stimulated overnight with the effector dose 50 (ED50) of exogeneous IL-12, IL-15 and IL-21 cytokines. The percentage viable target cells are identified as Annexin V⁻ and 7-AAD⁻ cells with flow cytometry. The baseline conditions represent the viability of the CD70⁺ target cells without the effector cells. Experiments were performed at least in triplicate. Errors bars represent standard deviation. * P≤0.05.

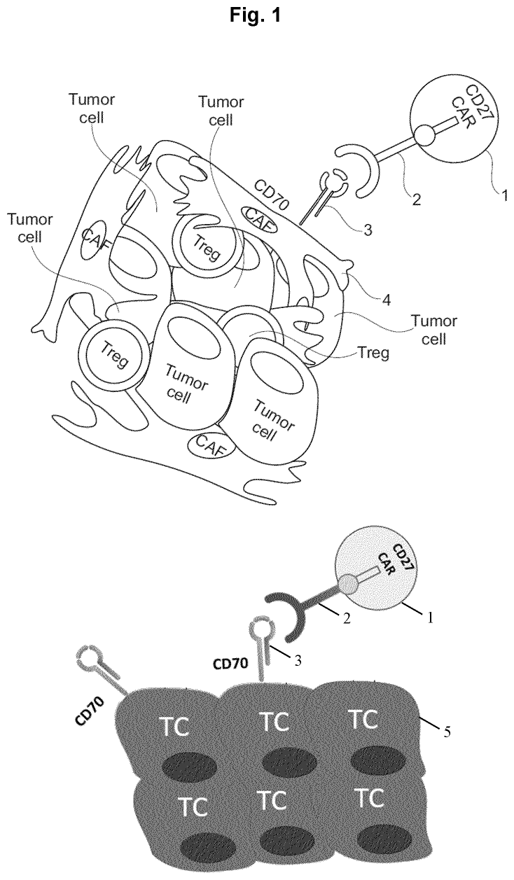

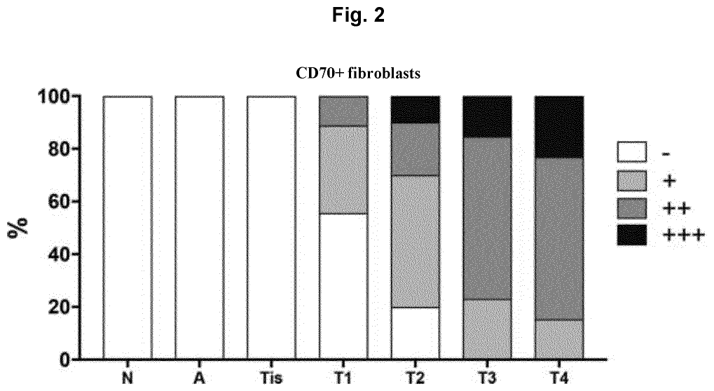

### DESCRIPTION OF EMBODIMENTS

As used herein, the singular forms “a”, “an”, and “the” include both singular and plural referents unless the context clearly dictates otherwise.

The terms “comprising”, “comprises” and “comprised of” as used herein are synonymous with “including”, “includes” or “containing”, “contains”, and are inclusive or open-ended and do not exclude additional, non-recited members, elements or method steps. The terms also encompass “consisting of” and “consisting essentially of”, which enjoy well-established meanings in patent terminology.

The recitation of numerical ranges by endpoints includes all numbers and fractions subsumed within the respective ranges, as well as the recited endpoints. This applies to numerical ranges irrespective of whether they are introduced by the expression “from . . . to . . . ” or the expression “between . . . and . . . ” or another expression.

The terms “about” or “approximately” as used herein when referring to a measurable value such as a parameter, an amount, a temporal duration, and the like, are meant to encompass variations of and from the specified value, such as variations of +/−10% or less, preferably +/−5% or less, more preferably +/−1% or less, and still more preferably +/−0.1% or less of and from the specified value, insofar such variations are appropriate to perform in the disclosed invention. It is to be understood that the value to which the modifier “about” or “approximately” refers is itself also specifically, and preferably, disclosed.

Whereas the terms “one or more” or “at least one”, such as one or more members or at least one member of a group of members, is clear per se, by means of further exemplification, the term encompasses inter alia a reference to any one of said members, or to any two or more of said members, such as, e.g., any ≥3, ≥4, ≥5, ≥6 or ≥7 etc. of said members, and up to all said members. In another example, “one or more” or “at least one” may refer to 1, 2, 3, 4, 5, 6, 7 or more.

The discussion of the background to the invention herein is included to explain the context of the invention. This is not to be taken as an admission that any of the material referred to was published, known, or part of the common general knowledge in any country as of the priority date of any of the claims.

Throughout this disclosure, various publications, patents and published patent specifications are referenced by an identifying citation. All documents cited in the present specification are hereby incorporated by reference in their entirety. In particular, the teachings or sections of such documents herein specifically referred to are incorporated by reference.

Unless otherwise defined, all terms used in disclosing the invention, including technical and scientific terms, have the meaning as commonly understood by one of ordinary skill in the art to which this invention belongs. By means of further guidance, term definitions are included to better appreciate the teaching of the invention. When specific terms are defined in connection with a particular aspect of the invention or a particular embodiment of the invention, such connotation or meaning is meant to apply throughout this specification, i.e., also in the context of other aspects or embodiments of the invention, unless otherwise defined.

In the following passages, different aspects or embodiments of the invention are defined in more detail. Each aspect or embodiment so defined may be combined with any other aspect(s) or embodiment(s) unless clearly indicated to the contrary. In particular, any feature indicated as being preferred or advantageous may be combined with any other feature or features indicated as being preferred or advantageous.

Reference throughout this specification to “one embodiment”, “an embodiment” means that a particular feature, structure or characteristic described in connection with the embodiment is included in at least one embodiment of the present invention. Thus, appearances of the phrases “in one embodiment” or “in an embodiment” in various places throughout this specification are not necessarily all referring to the same embodiment, but may. Furthermore, the particular features, structures or characteristics may be combined in any suitable manner, as would be apparent to a person skilled in the art from this disclosure, in one or more embodiments. Furthermore, while some embodiments described herein include some, but not other features included in other embodiments, combinations of features of different embodiments are meant to be within the scope of the invention, and form different embodiments, as would be understood by those in the art. For example, in the appended claims, any of the claimed embodiments can be used in any combination.

As corroborated by the experimental section, which illustrates certain representative embodiments of the present invention, the inventors have demonstrated the feasibility and therapeutic usefulness of targeting CD70 positive cancer cells and/or CD70 positive tumor microenvironment cells such as cancer associated fibroblasts (CAFs) using natural killer (NK) cells engineered to express a chimeric antigen receptor (CAR) which comprises the extracellular domain of CD27 or a CD70-binding portion thereof. Efficient eradication of CD70 positive cancer cells and/or CAFs provides a valuable therapeutic avenue for the treatment of proliferative diseases.

The present invention thus encompasses aspects as set forth in the Summary section, in particular pertaining to an NK cell engineered to express a CAR, wherein the CAR comprises the extracellular domain of CD27 or a CD70-binding portion thereof; as well as to pharmaceutical compositions comprising the so engineered NK cells, to methods for producing the engineered NK cells, and to therapeutic uses and methods employing the engineered NK cells or the pharmaceutical compositions, in particular for the treatment of neoplastic diseases, such as cancer.

The term “Chimeric Antigen Receptor” or alternatively a “CAR” refers to a recombinant polypeptide or a set of polypeptides, which, when expressed by an immune effector cell, endows the cell with specificity for a target antigen on the surface of a target cell, and with intracellular signal transduction. In some embodiments, a CAR comprises at least an extracellular antigen binding domain, a transmembrane domain and a cytoplasmic signalling domain (also referred to herein as “an intracellular signalling domain” or “an intracellular activation domain”) comprising a functional signalling domain derived from a stimulatory molecule and/or a costimulatory molecule. The term “signalling domain” refers to the functional portion of a protein which acts by transmitting information within the cell to regulate cellular activity via defined signalling pathways by generating second messengers or functioning as effectors by responding to such messengers. Typically, a CAR may comprise a chimeric fusion protein, such that for example an antigen binding domain and an intracellular signalling domain are comprised within the same polypeptide chain. In alternative embodiments, a CAR may be formed by a set of polypeptides not contiguous with each other, such that for example an antigen binding domain and an intracellular signalling domain may be provided in separate polypeptide chains, configured to heterodimerise to form the CAR. By means of illustration, the antigen binding domain and the intracellular signalling domain may each be provided with a dimerization switch that, upon the presence of a dimerization molecule, can couple the polypeptides containing said domains to one another.

While the term CAR constitutes an appropriate, convenient and well-established manner to denote receptors such as those contemplated herein, one could alternatively refer to such receptors without using this term, e.g., as a polypeptide or a set of polypeptides comprising the extracellular domain of CD27 or a CD70-binding portion thereof and an intracellular activation domain, whereby binding of the extracellular domain to CD70 on the surface of a target cell elicits, induces or leads to intracellular signal generation through the intracellular activation domain.

Alternative CAR constructs may typically be characterised as belonging to successive generations. In first-generation CARs the intracellular signalling domain contains or consists essentially of the zeta chain associated with the T cell receptor complex (CD3ζ) or the γ subunit of the immunoglobulin Fc receptor (FcRγ). The cytoplasmic signalling domain of second-generation CARs further comprises an intracellular costimulatory domain, i.e., a functional signalling domain derived from at least one costimulatory molecule, such as CD28, 4-1BB (CD137), DAP10, ICOS, or OX40 (CD134), and third-generation CARS include a combination of two or more such costimulatory endodomains.

In certain embodiments, the CAR comprises a chimeric fusion protein comprising the extracellular domain of CD27 or a CD70-binding portion thereof, a transmembrane domain and an intracellular signalling domain comprising a functional signalling domain derived from a stimulatory molecule. In certain embodiments, the CAR comprises a chimeric fusion protein comprising the extracellular domain of CD27 or a CD70-binding portion thereof, a transmembrane domain and an intracellular signalling domain comprising a functional signalling domain derived from a costimulatory molecule and a functional signalling domain derived from a stimulatory molecule. In certain embodiments, the CAR comprises a chimeric fusion protein comprising the extracellular domain of CD27 or a CD70-binding portion thereof, a transmembrane domain and an intracellular signalling domain comprising two functional signalling domains derived from one or more costimulatory molecule(s) and a functional signalling domain derived from a stimulatory molecule. In certain embodiments, the CAR comprises a chimeric fusion protein comprising the extracellular domain of CD27 or a CD70-binding portion thereof, a transmembrane domain and an intracellular signalling domain comprising at least two functional signalling domains derived from one or more costimulatory molecule(s) and a functional signalling domain derived from a stimulatory molecule.

In certain embodiments, the intracellular portion of the CAR comprises at least one intracellular activation domain. In certain embodiments, the at least one intracellular activation domain is selected from the group consisting of a CD3ζ activation domain, a FcRγ activation domain, and combinations thereof. In certain preferred embodiments, the CAR comprises the CD3ζ intracellular activation domain, such as wherein the intracellular activation domain of the CAR comprises, consists essentially of or consists of the CD3ζ intracellular activation domain. In certain embodiments, the intracellular portion of the CAR comprises at least one intracellular costimulatory domain. In certain embodiments, at least one intracellular costimulatory domain is selected from the group consisting of a CD28 costimulatory domain, a 4-1BB costimulatory domain, a DAP10 costimulatory domain, a OX40 costimulatory domain, an ICOS costimulatory domain, and combinations thereof. In certain preferred embodiments, the CAR comprises the 4-1BB intracellular co-stimulatory domain, and optionally one or more additional co-stimulatory domains. In certain preferred embodiments, the CAR comprises the 4-1BB intracellular co-stimulatory domain and does not comprise another co-stimulatory domain.

As noted, the CAR comprises the extracellular domain of CD27 or a CD70-binding portion thereof. Cluster Of Differentiation 27 (CD27) molecule, or CD27 or CD27 antigen in short, also known as CD27L receptor or tumor necrosis factor receptor superfamily member 7 (TNFRSF7 protein), is a 29-kDa single-pass type I membrane glycoprotein. Human CD27 precursor is annotated under U.S. government's National Center for Biotechnology Information (NCBI) Genbank (http://www.ncbi.nlm.nih.gov/) Gene ID no. 939. A human wild-type CD27 amino acid sequence may be as annotated under Genbank accession no: NP_001233.2 or Swissprot/Uniprot (http://www.uniprot.org/) accession no: P26842-1 (entry version 195, 7 Oct. 2020, sequence version 2, 24 Nov. 2009), the NP_001233.2 sequence reproduced here below (the signal or leader sequence, the N-terminal extracellular domain, the transmembrane or intramembrane domain, and the C-terminal intracellular domain of the CD27 molecule as annotated in the aforementioned database entry are shown in underlined, standard, bold, and italics fonts, consecutively and respectively):

(SEQ ID NO: 2)
MARPHPWWLCVLGTLVGLSATPAPKSCPERHYWAQGKLCCQMCEPGTFLV
KDCDQHRKAAQCDPCIPGVSFSPDHHTRPHCESCRHCNSGLLVRNCTITA
NAECACRNGWQCRDKECTECDPLPNPSLTARSSQALSPHPQPTHLPYVSE
MLEARTAGHMQTLADFRQLPARTLSTHWPPQRSLCSSDFIRILVIFSGMF
LVFTLAGALFLHQRRKYRSNKGESPVEPAEPCHYSCPREEEGSTIPIQED
YRKPEPACSP.

CD27 as intended herein may particularly concern human CD27. The qualifier “human” as used herein in connection with a CD27 protein may particularly refer to the amino acid sequence of the CD27 protein. For example, a CD27 protein having the amino acid sequence as a CD27 protein found in humans may also be obtained by technical means, e.g., by recombinant expression, cell-free translation, or non-biological peptide synthesis. A skilled person understands that the amino acid sequence of a given native protein such as a CD27 protein may differ between or within different individuals of the same species due to normal genetic diversity (allelic variation, polymorphism) within that species and/or due to differences in post-transcriptional or post-translational modifications. Any such variants or isoforms of the native protein are subsumed by the reference to or designation of the protein.

The extracellular domain of the human CD27 protein as annotated under Genbank accession no: NP_001233.2 is reproduced below:

(SEQ ID NO: 3)
ATPAPKSCPERHYWAQGKLCCQMCEPGTFLVKDCDQHRKAAQCDPCIPGV
SFSPDHHTRPHCESCRHCNSGLLVRNCTITANAECACRNGWQCRDKECTE
CDPLPNPSLTARSSQALSPHPQPTHLPYVSEMLEARTAGHMQTLADFRQL
PARTLSTHWPPQRSLCSSDFIR.

In certain embodiments, the extracellular domain of CD27 denotes a polypeptide comprising, consisting essentially of or consisting of an amino acid sequence at least 80% identical to SEQ ID NO: 3, such as at least 85% identical to SEQ ID NO: 3, preferably at least 90% identical to SEQ ID NO: 3, such as at least 95% identical to SEQ ID NO: 3, more preferably at least 96%, at least 97%, at least 98%, or at least 99% identical to SEQ ID NO: 3. In particularly preferred embodiments, the extracellular domain of CD27 comprises, consists essentially of or consists of the amino acid sequence set forth in SEQ ID NO: 3.

The term “sequence identity” with regard to amino acid sequences denotes the extent of overall sequence identity (i.e., including the whole or entire amino acid sequences as recited in the comparison) expressed in % between the amino acid sequences read from N-terminus to C-terminus; and with regard to nucleic acid sequences the extent of overall sequence identity (i.e., including the whole or entire nucleic acid sequences as recited in the comparison) expressed in % between the nucleic acid sequences read from 5′-terminus to 3′-terminus (optionally, complementary sequences may be used in the comparison). Sequence identity may be determined using suitable algorithms for performing sequence alignments and determination of sequence identity as know per se. Exemplary but non-limiting algorithms include those based on the Basic Local Alignment Search Tool (BLAST) originally described by Altschul et al. 1990 (J Mol Biol 215: 403-10), such as the “Blast 2 sequences” algorithm described by Tatusova and Madden 1999 (FEMS Microbiol Lett 174: 247-250), for example using the published default settings or other suitable settings (such as, e.g., for the BLASTN algorithm: cost to open a gap=5, cost to extend a gap=2, penalty for a mismatch=−2, reward for a match=1, gap x_dropoff=50, expectation value=10.0, word size=28; or for the BLASTP algorithm: matrix=Blosum62 (Henikoff et al., 1992, Proc. Natl. Acad. Sci., 89:10915-10919), cost to open a gap=11, cost to extend a gap=1, expectation value=10.0, word size=3).

An example procedure to determine the percent identity between a particular amino acid sequence and a query amino acid sequence will entail aligning the two amino acid sequences each read from N-terminus to C-terminus using the Blast 2 sequences (B12seq) algorithm, available as a web application (https://blast.ncbi.nlm nih gov/Blast.cgi?PROGRAM=blastp&PAGE_TYPE=BlastSearch&BLAST_SPEC=blast2seq&LINK_LOC=blasttab) or as a standalone executable programme (BLAST version 2.11.0+) at the NCBI web site (https://flp.ncbi.nlm.nih.gov/blast/executables/blast+/LATEST/), using suitable algorithm parameters. An example of suitable algorithm parameters includes: matrix=Blosum62, cost to open a gap=11, cost to extend a gap=1, expectation value=10.0, word size=3). If the two compared sequences share identity, then the output will present those regions of identity as aligned sequences. If the two compared sequences do not share identity, then the output will not present aligned sequences. Once aligned, the number of matches will be determined by counting the number of positions where an identical amino acid residue is presented in both sequences. The percent identity is determined by dividing the number of matches by the length of the query sequence, followed by multiplying the resulting value by 100. The percent identity value may, but need not, be rounded to the nearest tenth. For example, 78.11, 78.12, 78.13, and 78.14 may be rounded down to 78.1, while 78.15, 78.16, 78.17, 78.18, and 78.19 may be rounded up to 78.2. It is further noted that the detailed view for each segment of alignment as outputted by B12seq already conveniently includes the percentage of identities.

Where an amino acid sequence differs, varies or diverges from a certain other amino acid sequence—for example, where the former amino acid sequence is said to display a certain degree or percentage of sequence identity to the latter amino acid sequence, or where the former amino acid sequence is said to differ by a certain number of amino acids from the latter amino acid sequence—such sequence variation may be constituted by one or more amino acid additions (e.g., a single amino acid or a stretch of two or more contiguous amino acids may be added at one position of an amino acid sequence or each independently at two or more positions of an amino acid sequence), deletions (e.g., a single amino acid or a stretch of two or more contiguous amino acids may be deleted at one position of an amino acid sequence or each independently at two or more positions of an amino acid sequence), and/or or substitutions (e.g., a single amino acid or a stretch of two or more contiguous amino acids may substitute a single one or a stretch of two or more contiguous amino acids at one position of an amino acid sequence or each independently at two or more positions of an amino acid sequence).

Preferably, the one or more amino acid substitutions, in particular one or more single amino acid substitutions, may be conservative amino acid substitutions. A conservative amino acid substitution is a substitution of one amino acid for another with similar characteristics. Conservative amino acid substitutions include substitutions within the following groups: valine, alanine and glycine; leucine, valine, and isoleucine; aspartic acid and glutamic acid; asparagine and glutamine; serine, cysteine, and threonine; lysine and arginine; and phenylalanine and tyrosine. The nonpolar hydrophobic amino acids include alanine, leucine, isoleucine, valine, proline, phenylalanine, tryptophan and methionine. The polar neutral amino acids include glycine, serine, threonine, cysteine, tyrosine, asparagine and glutamine. The positively charged (i.e., basic) amino acids include arginine, lysine and histidine. The negatively charged (i.e., acidic) amino acids include aspartic acid and glutamic acid. Any substitution of one member of the above-mentioned polar, basic, or acidic groups by another member of the same group can be deemed a conservative substitution. By contrast, a non-conservative substitution is a substitution of one amino acid for another with dissimilar characteristics.

The present specification discusses certain molecules which may be peptides, polypeptides or proteins, such as for example, the extracellular domain of CD27, or the intracellular activation domain. The term “protein” generally encompasses macromolecules comprising one or more polypeptide chains. The term “polypeptide” generally encompasses linear polymeric chains of amino acid residues linked by peptide bonds. A “peptide bond”, “peptide link” or “amide bond” is a covalent bond formed between two amino acids when the carboxyl group of one amino acid reacts with the amino group of the other amino acid, thereby releasing a molecule of water. Especially when a protein is only composed of a single polypeptide chain, the terms “protein” and “polypeptide” may be used interchangeably to denote such a protein. The terms are not limited to any minimum length of the polypeptide chain. Polypeptide chains consisting essentially of or consisting of 50 or less (≤50) amino acids, such as ≤45, ≤40, ≤35, ≤30, ≤25, ≤20, ≤15, ≤10 or ≤5 amino acids may be commonly denoted as a “peptide”. In the context of proteins, polypeptides or peptides, a “sequence” is the order of amino acids in the chain in an amino to carboxyl terminal direction in which residues that neighbour each other in the sequence are contiguous in the primary structure of the protein, polypeptide or peptide. The terms may encompass naturally, recombinantly, semi-synthetically or synthetically produced proteins, polypeptides or peptides. Hence, for example, a protein, polypeptide or peptide can be present in or isolated from nature, e.g., produced or expressed natively or endogenously by a cell or tissue and optionally isolated therefrom; or a protein, polypeptide or peptide can be recombinant, i.e., produced by recombinant DNA technology, and/or can be, partly or entirely, chemically or biochemically synthesised. Without limitation, a protein, polypeptide or peptide can be produced recombinantly by a suitable host or host cell expression system and optionally isolated therefrom (e.g., a suitable bacterial, yeast, fungal, plant or animal host or host cell expression system), or produced recombinantly by cell-free translation or cell-free transcription and translation, or non-biological peptide, polypeptide or protein synthesis. The terms also encompasses proteins, polypeptides or peptides that carry one or more co- or post-expression-type modifications of the polypeptide chain(s), such as, without limitation, glycosylation, lipidation, acetylation, amidation, phosphorylation, sulphonation, methylation, pegylation (covalent attachment of polyethylene glycol typically to the N-terminus or to the side-chain of one or more Lys residues), ubiquitination, sumoylation, cysteinylation, glutathionylation, oxidation of methionine to methionine sulphoxide or methionine sulphone, signal peptide removal, N-terminal Met removal, conversion of pro-enzymes or pre-hormones into active forms, etc. Such co- or post-expression-type modifications may be introduced in vivo by a host cell expressing the proteins, polypeptides or peptides (co- or post-translational protein modification machinery may be native to the host cell and/or the host cell may be genetically engineered to comprise one or more (additional) co- or post-translational protein modification functionalities), or may be introduced in vitro by chemical (e.g., pegylation) and/or biochemical (e.g., enzymatic) modification of the isolated proteins, polypeptides or peptides.

The term “amino acid” encompasses naturally occurring amino acids, naturally encoded amino acids, non-naturally encoded amino acids, non-naturally occurring amino acids, amino acid analogues and amino acid mimetics that function in a manner similar to the naturally occurring amino acids, all in their D- and L-stereoisomers, provided their structure allows such stereoisomeric forms. Amino acids are referred to herein by either their name, their commonly known three letter symbols or by the one-letter symbols recommended by the IUPAC-IUB Biochemical Nomenclature Commission. A “naturally encoded amino acid” refers to an amino acid that is one of the 20 common amino acids or pyrrolysine, pyrroline-carboxy-lysine or selenocysteine. The 20 common amino acids are: Alanine (A or Ala), Cysteine (C or Cys), Aspartic acid (D or Asp), Glutamic acid (E or Glu), Phenylalanine (F or Phe), Glycine (G or Gly), Histidine (H or His), Isoleucine (I or Ile), Lysine (K or Lys), Leucine (L or Leu), Methionine (M or Met), Asparagine (N or Asn), Proline (P or Pro), Glutamine (Q or Gln), Arginine (R or Arg), Serine (S or Ser), Threonine (T or Thr), Valine (V or Val), Tryptophan (W or Trp), and Tyrosine (Y or Tyr). A “non-naturally encoded amino acid” refers to an amino acid that is not one of the 20 common amino acids or pyrrolysine, pyrroline-carboxy-lysine or selenocysteine. The term includes without limitation amino acids that occur by a modification (such as a post-translational modification) of a naturally encoded amino acid, but are not themselves naturally incorporated into a growing polypeptide chain by the translation complex, as exemplified without limitation by N-acetylglucosaminyl-L-serine, N-acetylglucosaminyl-L-threonine, and O-phosphotyrosine. Further examples of non-naturally encoded, un-natural or modified amino acids include 2-Aminoadipic acid, 3-Aminoadipic acid, beta-Alanine, beta-Aminopropionic acid, 2-Aminobutyric acid, 4-Aminobutyric acid, piperidinic acid, 6-Aminocaproic acid, 2-Aminoheptanoic acid, 2-Aminoisobutyric acid, 3-Aminoisobutyric acid, 2-Aminopimelic acid, 2,4 Diaminobutyric acid, Desmosine, 2,2′-Diaminopimelic acid, 2,3-Diaminopropionic acid, N-Ethylglycine, N-Ethylasparagine, homoserine, homocysteine, Hydroxylysine, allo-Hydroxylysine, 3-Hydroxyproline, 4-Hydroxyproline, Isodesmosine, allo-Isoleucine, N-Methylglycine, N-Methylisoleucine, 6-N-Methyllysine, N-Methylvaline, Norvaline, Norleucine, or Ornithine. Also included are amino acid analogues, in which one or more individual atoms have been replaced either with a different atom, an isotope of the same atom, or with a different functional group. Also included are un-natural amino acids and amino acid analogues described in Ellman et al. Methods Enzymol. 1991, vol. 202, 301-36. The incorporation of non-natural amino acids into proteins, polypeptides or peptides may be advantageous in a number of different ways. For example, D-amino acid-containing proteins, polypeptides or peptides exhibit increased stability in vitro or in vivo compared to L-amino acid-containing counterparts. More specifically, D-amino acid-containing proteins, polypeptides or peptides may be more resistant to endogenous peptidases and proteases, thereby providing improved bioavailability of the molecule and prolonged lifetimes in vivo.

In certain embodiments, the CAR may comprise a CD70-binding portion or fragment of the extracellular domain of CD27. The terms “fragment” or “portion” of a protein, polypeptide or peptide generally encompass N-terminally and/or C-terminally deleted or truncated forms of the full-length protein, polypeptide or peptide. The term encompasses fragments arising by any mechanism, such as, without limitation, by expression of a truncated form of the full-length protein, polypeptide or peptide, or by physical, chemical or enzymatic proteolysis of the full-length protein, polypeptide or peptide in vivo or in vitro. Without limitation, a fragment of a protein, polypeptide or peptide may represent at least about 50% (by amino acid number), e.g., at least about 60%, or at least about 70%, or at least about 80%, or at least about 90%, or at least about 91%, or at least about 92%, or at least about 93%, or at least about 94%, or at least about 95%, or at least about 96%, or at least about 97%, or at least about 98%, or at least about 99% of the contiguous amino acid sequence of the full-length protein, polypeptide or peptide. For example, a fragment or portion of the illustrative 172 amino acid-long CD27 extracellular domain as set forth in SEQ ID NO: 3 may include a sequence of at least about 90, or at least about 100, or at least about 110, or at least about 120, or at least about 130, or at least about 140, or at least about 150, or at least about 160, or at least about 170 consecutive amino acids of SEQ ID NO: 3.

In nature, CD27 is a cognate receptor for the CD70 molecules expressed on cells. Accordingly, the extracellular domain of CD27 comprised by the CAR as taught herein has the intrinsic capacity to specifically bind to the CD70 protein on the surface of cells in physiologically and therapeutically relevant settings. The term “specifically bind” as used throughout this specification means that an agent binds to one or more desired molecules or analytes substantially to the exclusion of other molecules which are random or unrelated, and optionally substantially to the exclusion of other molecules that are structurally related. Put differently, an agent is said to specifically bind a target molecule when it preferentially recognises its target molecule in a complex mixture of proteins and/or macromolecules.

When the CAR includes a CD70-binding portion or fragment of the extracellular domain of CD27, the capacity of such CD27 portion to bind to the CD70 protein is meaningfully similar or comparable to the ability of the full-length CD27 extracellular domain to bind to CD70, such that cells expressing CARs comprising the CD70-binding portion of the CD27 extracellular domain will exhibit a therapeutic effect meaningfully similar or comparable to the therapeutic effect achieved by cells expressing otherwise identical CARs comprising the full-length CD27 extracellular domain.

Typically, target-binding proteins (such as the extracellular domain of CD27 or the CD70-binding fragment thereof) may bind to their cognate target proteins (such as CD70) with a dissociation constant (KD) of 1×10⁻⁵ to 1×10⁻¹² moles/liter (M) or less, and preferably 1×10⁻⁷ to 1×10⁻¹² M or less, and more preferably 1×10⁻⁸ to 1×10⁻¹² M or less and even more preferably 1×10⁻⁹ to 1×10⁻¹² M or less, such as between 1×10⁻⁹ and 1×10⁻¹⁰ M, or between 1×10⁻¹⁰ and 1×10⁻¹¹ M, wherein KD=[TBP][TG]/[TBP-TG], TBP denotes the target binding protein, TP denotes the target protein, and TBP-TG denotes the complex of the two. KD values greater than 10⁻⁴ M are typically considered as indicative of non-specific binding. Hence, in certain embodiments, the CD70-binding portion of the CD27 extracellular domain may bind to CD70 with KD of 10⁻⁴ M or less, preferably 10⁻⁵ M or less, more preferably 10⁻⁶ M or less, even more preferably 10⁻⁷ M or less, still more preferably 10⁻⁸ M or less, such as 10⁻⁹ M or less, 10⁻¹⁰ M or less, or 10⁻¹¹ M or less. In certain embodiments, the CD70-binding portion of the CD27 extracellular domain may bind to CD70 with KD which is at most 2 orders of magnitude higher, preferably at most 1 order of magnitude higher, more preferably the same order of magnitude as or one or more magnitudes lower than the KD typifying the binding of the full-length CD27 extracellular domain to CD70 under otherwise substantially identical conditions. Specific binding of a target-binding protein to a target can be determined in any suitable manner known per se, including, for example, Scatchard plot analysis and/or competitive binding assays, such as radioimmunoassays (RIA), enzyme immunoassays (EIA) and sandwich competition assays, and the different variants thereof known per se in the art.

In certain embodiments, the suitability of a given portion or fragment of the CD27 extracellular domain in the therapeutic context of the present invention, may be evaluated by in vitro cell killing experiments, such as illustrated in the Examples. For instance, where NK cells expressing a CAR comprising a given portion of the CD27 extracellular domain displays at 10% of the efficiency of an NK cell expressing an otherwise identical CAR comprising the full-length CD27 extracellular domain in killing CD70 positive cells under otherwise substantially identical conditions, said portion of the CD27 extracellular domain may be deemed CD70-binding as intended herein. Preferably, the killing efficiency of the NK cells expressing the CAR comprising the CD70-binding portion of the CD27 extracellular domain may be at least 20%, such as at least 30% or at least 40%, more preferably at least 50%, such as at least 60% or at least 70%, even more preferably at least 80% such as at least 90%, or may be substantially comparable to or even higher than the killing efficiency of the NK cells expressing the CAR comprising the full-length CD27 extracellular domain.

In certain embodiments, the CAR may further comprise the intramembrane domain of CD27. The intramembrane or transmembrane domain of the human CD27 protein as annotated under Genbank accession no: NP_001233.2 is reproduced below:

| (SEQ ID NO: 4) | |
|---|---|
| | ILVIFSGMFLVFTLAGALFLH. |

In certain embodiments, the intramembrane domain of CD27 denotes a polypeptide comprising, consisting essentially of or consisting of an amino acid sequence at least 80% identical to SEQ ID NO: 4, such as at least 85% identical to SEQ ID NO: 4, preferably at least 90% identical to SEQ ID NO: 4, such as at least 95% identical to SEQ ID NO: 4, more preferably at least 96%, at least 97%, at least 98%, or at least 99% identical to SEQ ID NO: 4. In particularly preferred embodiments, the intramembrane domain of CD27 comprises, consists essentially of or consists of the amino acid sequence set forth in SEQ ID NO: 4.

A polypeptide consisting of the extracellular and intramembrane domains of the human CD27 protein as annotated under Genbank accession no: NP_001233.2 is as reproduced below:

(SEQ ID NO: 10)
ATPAPKSCPERHYWAQGKLCCQMCEPGTFLVKDCDQHRKAAQCDPCIPGV
SFSPDHHTRPHCESCRHCNSGLLVRNCTITANAECACRNGWQCRDKECTE
CDPLPNPSLTARSSQALSPHPQPTHLPYVSEMLEARTAGHMQTLADFRQL
PARTLSTHWPPQRSLCSSDFIRILVIFSGMFLVFTLAGALFLH.

In certain embodiments, the extracellular and intramembrane domains of CD27 denote a polypeptide comprising, consisting essentially of or consisting of an amino acid sequence at least 80% identical to SEQ ID NO: 10, such as at least 85% identical to SEQ ID NO: 10, preferably at least 90% identical to SEQ ID NO: 10, such as at least 95% identical to SEQ ID NO: 10, more preferably at least 96%, at least 97%, at least 98%, or at least 99% identical to SEQ ID NO: 10.

In particularly preferred embodiments, the extracellular and intramembrane domains of CD27 comprise, consist essentially of or consist of the amino acid sequence set forth in SEQ ID NO: 10.

In certain other embodiments, the CAR may comprise an intramembrane domain derived from a transmembrane protein, preferably a single-pass type I transmembrane protein, other than CD27. By means of an example and without limitation, many CAR constructs in the art use CD8α transmembrane domain, optionally with a CD8a hinge domain, and this/these can also be adopted in the present CAR molecules. In yet other embodiments, the CAR may comprise a non-naturally occurring or synthetic intramembrane domain engineered based on biophysical criteria known to apply to this type of transmembrane domains, such as, for example, high hydrophobicity and alpha-helical primary structure.

In certain embodiments, the CAR lacks all or a portion of the intracellular domain of CD27. The intracellular or cytoplasmic domain of the human CD27 protein as annotated under Genbank accession no: NP_001233.2 is reproduced below:

(SEQ ID NO: 5)
QRRKYRSNKGESPVEPAEPCHYSCPREEEGSTIPIQEDYRKPEPACSP.

In preferred embodiments, the CAR lacks all of the intracellular domain of CD27, i.e., the CAR lacks the entire intracellular domain of CD27, such as lacks all of the amino acid sequence set forth in SEQ ID NO: 5. In other embodiments the CAR may comprise a modified CD27 intracellular domain lacking at least 1, such as at least 2, at least 5, at least 10, at least 20, at least 30, or at least 40 contiguous or non-contiguous amino acids of the full-length CD27 intracellular domain. In particular embodiments, the CD27 intracellular domain may be modified such as to disable the normal CD27 signalling by said domain. Such modification may involve any amino acid sequence alteration, such as deletion and/or substitution of one or more amino acids of the CD27 intracellular domain.

Hence, in certain embodiments, the CAR comprises, consists essentially of or consists of the extracellular and intramembrane domains of CD27, and an intracellular activation domain. In other embodiments, the CAR comprises, consists essentially of or consists of the extracellular and intramembrane domains of CD27, an intracellular activation domain and an intracellular costimulatory domain. In further embodiments, the CAR comprises, consists essentially of or consists of the extracellular and intramembrane domains of CD27, an intracellular activation domain and two or more, such as precisely two, intracellular costimulatory domains. In certain embodiments, the CAR comprises, consists essentially of or consists of the extracellular and intramembrane domains of CD27, the CD3ζ intracellular activation domain, and an intracellular costimulatory domain. In certain embodiments, the CAR comprises, consists essentially of or consists of the extracellular and intramembrane domains of CD27, the CD3ζ intracellular activation domain, and two or more, such as precisely two, intracellular costimulatory domains. In particularly preferred embodiments, the CAR comprises, consists essentially of or consists of the extracellular and intramembrane domains of CD27, the CD3ζ intracellular activation domain, and the 4-1BB intracellular co-stimulatory domain.

One particular example of human CD3ζ intracellular activation domain is reproduced below:

(SEQ ID NO: 6)
RVKFSRSADAPAYKQGQNQLYNELNLGRREEYDVLDKRRGRDPEMGGKPR
RKNPQEGLYNELQKDKMAEAYSEIGMKGERRRGKGHDGLYQGLSTATKDT
YDALHMQALPPR.

In certain embodiments, the CD3ζ intracellular activation domain denotes a polypeptide comprising, consisting essentially of or consisting of an amino acid sequence at least 80% identical to SEQ ID NO: 6, such as at least 85% identical to SEQ ID NO: 6, preferably at least 90% identical to SEQ ID NO: 6, such as at least 95% identical to SEQ ID NO: 6, more preferably at least 96%, at least 97%, at least 98%, or at least 99% identical to SEQ ID NO: 6. In particularly preferred embodiments, the CD3ζ intracellular activation domain comprises, consists essentially of or consists of the amino acid sequence set forth in SEQ ID NO: 6.

One particular example of human 4-1BB intracellular co-stimulatory domain is reproduced below:

(SEQ ID NO: 7)

KRGRKKLLYIFKQPFMRPVQTTQEEDGCSCRFPEEEEGGCEL.

In certain embodiments, the 4-1BB intracellular co-stimulatory domain denotes a polypeptide comprising, consisting essentially of or consisting of an amino acid sequence at least 80% identical to SEQ ID NO: 7, such as at least 85% identical to SEQ ID NO: 7, preferably at least 90% identical to SEQ ID NO: 7, such as at least 95% identical to SEQ ID NO: 7, more preferably at least 96%, at least 97%, at least 98%, or at least 99% identical to SEQ ID NO: 7. In particularly preferred embodiments, the 4-1BB intracellular co-stimulatory domain comprises, consists essentially of or consists of the amino acid sequence set forth in SEQ ID NO: 7.

In certain particularly preferred embodiments, the CAR comprises, consists essentially of, or consists of the amino acid as set forth in SEQ ID NO: 1 below, with the extracellular and intramembrane domains of CD27 in standard font, the 4-1BB intracellular co-stimulatory domain in italics, and the CD3ζ intracellular activation domain underlined:

(SEQ ID NO: 1)
ATPAPKSCPERHYWAQGKLCCQMCEPGTFLVKDCDQHRKAAQCDPCIPGV
SFSPDHHTRPHCESCRHCNSGLLVRNCTITANAECACRNGWQCRDKECTE
CDPLPNPSLTARSSQALSPHPQPTHLPYVSEMLEARTAGHMQTLADFRQL
PARTLSTHWPPQRSLCSSDFIRILVIFSGMFLVFTLAGALFLHKRGRKKL
LYIFKQPFMRPVQTTQEEDGCSCRFPEEEEGGCELRVKFSRSADAPAYKQ
GQNQLYNELNLGRREEYDVLDKRRGRDPEMGGKPRRKNPQEGLYNELQKD
KMAEAYSEIGMKGERRRGKGHDGLYQGLSTATKDTYDALHMQALPPR

In certain embodiments the CAR comprises, consists essentially of or consists of an amino acid sequence at least 80% identical, preferably at least 85% identical, more preferably at least 90% identical, even more preferably at least 95% identical, such as particularly preferably at least 96%, at least 97%, at least 98% or at least 99% or 100% identical to SEQ ID NO: 1.

In certain embodiments the CAR comprises, consists essentially of or consists of (i) extracellular and intramembrane domains of CD27, (ii) a 4-1BB intracellular co-stimulatory domain, and (iii) a CD3ζ intracellular activation domain, wherein said domains (i)-(iii) each independently display at least 80% identity, preferably at least 85% identity, more preferably at least 90% identity, even more preferably at least 95% identity, such as particularly preferably at least 96%, at least 97%, at least 98% or at least 99% or 100% identity to (i) amino acid positions 1-193, (ii) amino acid positions 194-235, and (iii) 236-348 of SEQ ID NO: 1.

The skilled person shall appreciate that a CAR molecule as discussed here is a transmembrane protein and will typically require the inclusion of a suitable signal or leader sequence when expressed to effect the cellular membrane localisation of the protein. Such signal sequences are typically short (3-60 amino acids long) N-terminally located peptide chains, which are optionally and advantageously cleaved off or processed away by signal peptidase after the proteins are transported, such as to yield the mature protein. Signal sequences are widely known in the art and they may be applied for the expression of the CAR as taught herein. In certain embodiments, the signal sequence may be derived from a protein other than CD27. In certain preferred embodiments, the signal sequence may be derived from CD27. The native signal sequence of the human CD27 protein as annotated under Genbank accession no: NP_001233.2 is reproduced below:

| (SEQ ID NO: 8) | |
|---|---|
| | MARPHPWWLCVLGTLVGLS. |

In certain embodiments, the signal sequence comprised by the CAR molecule comprises, consists essentially of or consists of an amino acid sequence at least 80%, at least 90%, at least 95%, at least 10 96%, at least 97%, at least 98%, at least 99%, or 100% identical to SEQ ID NO: 8. In certain embodiments, such signal sequence can be N-terminally fused to any one of the CAR molecules individualised above.

Hence, by means of an illustration, in certain particularly preferred embodiments, a precursor form of the CAR comprises, consists essentially of, or consists of the amino acid as set forth in SEQ ID NO: 9 below, with the signal sequence in bold font, the extracellular and intramembrane domains of CD27 in standard font, the 4-1BB intracellular co-stimulatory domain in italics, and the CD3ζ intracellular activation domain underlined:

(SEQ ID NO: 9)
MARPHPWWLCVLGTLVGLSATPAPKSCPERHYWAQGKLCCQMCEPGTFLV
KDCDQHRKAAQCDPCIPGVSFSPDHHTRPHCESCRHCNSGLLVRNCTITA
NAECACRNGWQCRDKECTECDPLPNPSLTARSSQALSPHPQPTHLPYVSE
MLEARTAGHMQTLADFRQLPARTLSTHWPPQRSLCSSDFIRILVIFSGMF
LVFTLAGALFLHKRGRKKLLYIFKQPFMRPVQTTQEEDGCSCRFPEEEEG
GCELRVKFSRSADAPAYKQGQNQLYNELNLGRREEYDVLDKRRGRDPEMG
GKPRRKNPQEGLYNELQKDKMAEAYSEIGMKGERRRGKGHDGLYQGLSTA
TKDTYDALHMQALPPR.

Aspects of the invention also relate to any one CAR molecule as discussed in the present specification. Further aspects of the invention relate to nucleic acids encoding the CAR molecules as disclosed herein. By “encoding” is meant that a nucleic acid sequence or part(s) thereof corresponds, by virtue of the genetic code of an organism in question to a particular amino acid sequence, e.g., the amino acid sequence of one or more desired proteins or polypeptides, or to another nucleic acid sequence in a template-transcription product (e.g., RNA or RNA analogue) relationship.

The term “nucleic acid” as used herein typically refers to a polymer (preferably a linear polymer) of any length composed essentially of nucleoside units. A nucleoside unit commonly includes a heterocyclic base and a sugar group. Heterocyclic bases may include inter alia purine and pyrimidine bases such as adenine (A), guanine (G), cytosine (C), thymine (T) and uracil (U), which are widespread in naturally-occurring nucleic acids, other naturally-occurring bases (e.g., xanthine, inosine, hypoxanthine), as well as chemically or biochemically modified (e.g., methylated), non-natural or derivatised bases. Exemplary modified nucleobases include, without limitation, 5-substituted pyrimidines, 6-azapyrimidines and N-2, N-6 and 0-6 substituted purines, including 2-aminopropyladenine, 5-propynyluracil and 5-propynylcytosine. In particular, 5-methylcytosine substitutions have been shown to increase nucleic acid duplex stability. Sugar groups may include inter alia pentose (pentofuranose) groups such as preferably ribose and/or 2-deoxyribose common in naturally-occurring nucleic acids, or arabinose, 2-deoxyarabinose, threose or hexose sugar groups, as well as modified or substituted sugar groups (such as, without limitation, 2′-O-alkylated, e.g., 2′-O-methylated or 2′-O-ethylated sugars such as ribose; 2′-O-alkyloxyalkylated, e.g., 2′-O-methoxyethylated sugars such as ribose; or 2′-0,4′-C-alkylene-linked, e.g., 2′-O,4′-C-methylene-linked or 2′-O,4′-C-ethylene-linked sugars such as ribose; 2′-fluoro-arabinose, etc.). Nucleoside units may be linked to one another by any one of numerous known inter-nucleoside linkages, including inter alfa phosphodiester linkages common in naturally-occurring nucleic acids, and further modified phosphate- or phosphonate-based linkages such as phosphorothioate, alkyl phosphorothioate such as methyl phosphorothioate, phosphorodithioate, alkylphosphonate such as methylphosphonate, alkylphosphonothioate, phosphotriester such as alkylphosphotriester, phosphoramidate, phosphoropiperazidate, phosphoromorpholidate, bridged phosphoramidate, bridged methylene phosphonate, bridged phosphorothioate; and further siloxane, carbonate, sulfamate, carboalkoxy, acetamidate, carbamate such as 3′-N-carbamate, morpholino, borano, thioether, 3′-thioacetal, and sulfone internucleoside linkages. Preferably, inter-nucleoside linkages may be phosphate-based linkages including modified phosphate-based linkages, such as more preferably phosphodiester, phosphorothioate or phosphorodithioate linkages or combinations thereof. The term “nucleic acid” also encompasses any other nucleobase containing polymers such as nucleic acid mimetics, including, without limitation, peptide nucleic acids (PNA), peptide nucleic acids with phosphate groups (PHONA), locked nucleic acids (LNA), morpholino phosphorodiamidate-backbone nucleic acids (PMO), cyclohexene nucleic acids (CeNA), tricyclo-DNA (tcDNA), and nucleic acids having backbone sections with alkyl linkers or amino linkers (see, e.g., Kurreck 2003 (Eur J Biochem 270: 1628-1644)). “Alkyl” as used herein particularly encompasses lower hydrocarbon moieties, e.g., C₁-C₄ linear or branched, saturated or unsaturated hydrocarbon, such as methyl, ethyl, ethenyl, propyl, 1-propenyl, 2-propenyl, and isopropyl.

Nucleic acids as intended herein may include naturally occurring nucleosides, modified nucleosides or mixtures thereof. A modified nucleoside may include a modified heterocyclic base, a modified sugar moiety, a modified inter-nucleoside linkage or a combination thereof. The term “nucleic acid” further preferably encompasses DNA, RNA and DNA/RNA hybrid molecules, specifically including hnRNA, pre-mRNA, mRNA, cDNA, genomic DNA, amplification products, oligonucleotides, and synthetic (e.g., chemically synthesised) DNA, RNA or DNA/RNA hybrids. A nucleic acid can be naturally occurring, e.g., present in or isolated from nature, can be recombinant, i.e., produced by recombinant DNA technology, and/or can be, partly or entirely, chemically or biochemically synthesised. A “nucleic acid” can be double-stranded, partly double stranded, or single-stranded. Where single-stranded, the nucleic acid can be the sense strand or the antisense strand. In addition, nucleic acid can be circular or linear.

In certain embodiments, the extracellular and intramembrane domains of CD27, such as in particular those set forth in SEQ ID NO: 10, may be encoded by a nucleic acid comprising, consisting essentially of or consisting of the nucleic acid sequence set forth in SEQ ID NO: 11:

(SEQ ID NO: 11)
GCTACACCAGCTCCTAAGAGCTGCCCCGAGAGACACTATTGGGCCCAGGG
CAAGCTGTGCTGCCAGATGTGTGAACCTGGCACCTTCCTGGTCAAGGACT
GCGACCAGCACAGAAAGGCCGCTCAGTGCGATCCTTGTATCCCCGGCGTG
TCCTTCTCTCCCGACCACCACACAAGACCTCACTGCGAGAGCTGCAGACA
CTGCAATTCTGGACTGCTCGTGCGGAACTGCACCATCACAGCCAATGCCG
AGTGCGCCTGCAGAAATGGCTGGCAGTGCCGGGACAAAGAATGTACCGAG
TGCGACCCTCTGCCTAATCCTAGCCTGACCGCCAGAAGCAGCCAGGCTTT
GTCTCCTCATCCTCAGCCTACACATCTGCCCTACGTGTCCGAGATGCTGG
AAGCCAGAACAGCCGGCCATATGCAGACCCTGGCCGACTTTAGACAGCTG
CCCGCCAGAACACTGAGCACCCATTGGCCTCCACAGAGAAGCCTGTGCAG
CAGCGACTTCATCCGGATCCTCGTGATCTTCAGCGGCATGTTCCTGGTGT
TCACACTGGCTGGCGCCCTGTTTCTGCAT,

optionally wherein any codon in SEQ ID NO: 11 may each independently be replaced by another codon encoding the same amino acid, preferably wherein the nucleic acid sequence is thereby codon optimised for expression in cells of a desired species, such as in human cells, according to codon optimisation principles known in the art, such as wherein the extracellular and intramembrane domains of CD27 are encoded by a nucleic acid comprising, consisting essentially of or consisting of a nucleic acid sequence at least 60%, at least 70%, at least 80%, at least 90%, or at least 95% identical to SEQ ID NO: 11.

In certain embodiments, the human CD3ζ intracellular activation domain, such as in particular the one set forth in SEQ ID NO: 6, may be encoded by a nucleic acid comprising, consisting essentially of or consisting of the nucleic acid sequence set forth in SEQ ID NO: 12:

(SEQ ID NO: 12)
CGCGTGAAATTCAGCCGCAGCGCAGATGCTCCAGCCTACAAGCAGGGGCA
GAACCAGCTCTACAACGAACTCAATCTTGGTCGGAGAGAGGAGTACGACG
TGCTGGACAAGCGGAGAGGACGGGACCCAGAAATGGGCGGGAAGCCGCGC
AGAAAGAATCCCCAAGAGGGCCTGTACAACGAGCTCCAAAAGGATAAGAT
GGCAGAAGCCTATAGCGAGATTGGTATGAAAGGGGAACGCAGAAGAGGCA
AAGGCCACGACGGACTGTACCAGGGACTCAGCACCGCCACCAAGGACACC
TATGACGCTCTTCACATGCAGGCCCTGCCGCCTCGG,

optionally wherein any codon in SEQ ID NO: 12 may each independently be replaced by another codon encoding the same amino acid, preferably wherein the nucleic acid sequence is thereby codon optimised for expression in cells of a desired species, such as in human cells, according to codon optimisation principles known in the art, such as wherein the human CD3ζ intracellular activation domain is encoded by a nucleic acid comprising, consisting essentially of or consisting of a nucleic acid sequence at least 60%, at least 70%, at least 80%, at least 90%, or at least 95% identical to SEQ ID NO: 12.

In certain embodiments, the human 4-1BB intracellular co-stimulatory domain, such as in particular the one set forth in SEQ ID NO: 7, may be encoded by a nucleic acid comprising, consisting essentially of or consisting of the nucleic acid sequence set forth in SEQ ID NO: 13:

(SEQ ID NO: 13)
AAGCGCGGTCGGAAGAAGCTGCTGTACATCTTTAAGCAACCCTTCATGAG
GCCTGTGCAGACTACTCAAGAGGAGGACGGCTGTTCATGCCGGTTCCCAG
AGGAGGAGGAAGGCGGCTGCGAACTG,

optionally wherein any codon in SEQ ID NO: 13 may each independently be replaced by another codon encoding the same amino acid, preferably wherein the nucleic acid sequence is thereby codon optimised for expression in cells of a desired species, such as in human cells, according to codon optimisation principles known in the art, such as wherein the human 4-1BB intracellular co-stimulatory domain is encoded by a nucleic acid comprising, consisting essentially of or consisting of a nucleic acid sequence at least 60%, at least 70%, at least 80%, at least 90%, or at least 95% identical to SEQ ID NO: 13.

In certain embodiments, the precursor form of the CAR as taught herein, such as in particular the one set forth in SEQ ID NO: 9, may be encoded by a nucleic acid comprising, consisting essentially of or consisting of the nucleic acid sequence set forth in SEQ ID NO: 14:

(SEQ ID NO: 14)
ATGGCCAGACCTCATCCTTGGTGGCTGTGTGTGCTGGGCACACTCGTTGG
CCTGTCTGCTACACCAGCTCCTAAGAGCTGCCCCGAGAGACACTATTGGG
CCCAGGGCAAGCTGTGCTGCCAGATGTGTGAACCTGGCACCTTCCTGGTC
AAGGACTGCGACCAGCACAGAAAGGCCGCTCAGTGCGATCCTTGTATCCC
CGGCGTGTCCTTCTCTCCCGACCACCACACAAGACCTCACTGCGAGAGCT
GCAGACACTGCAATTCTGGACTGCTCGTGCGGAACTGCACCATCACAGCC
AATGCCGAGTGCGCCTGCAGAAATGGCTGGCAGTGCCGGGACAAAGAATG
TACCGAGTGCGACCCTCTGCCTAATCCTAGCCTGACCGCCAGAAGCAGCC
AGGCTTTGTCTCCTCATCCTCAGCCTACACATCTGCCCTACGTGTCCGAG
ATGCTGGAAGCCAGAACAGCCGGCCATATGCAGACCCTGGCCGACTTTAG
ACAGCTGCCCGCCAGAACACTGAGCACCCATTGGCCTCCACAGAGAAGCC
TGTGCAGCAGCGACTTCATCCGGATCCTCGTGATCTTCAGCGGCATGTTC
CTGGTGTTCACACTGGCTGGCGCCCTGTTTCTGCATAAGCGCGGTCGGAA
GAAGCTGCTGTACATCTTTAAGCAACCCTTCATGAGGCCTGTGCAGACTA
CTCAAGAGGAGGACGGCTGTTCATGCCGGTTCCCAGAGGAGGAGGAAGGC
GGCTGCGAACTGCGCGTGAAATTCAGCCGCAGCGCAGATGCTCCAGCCTA
CAAGCAGGGGCAGAACCAGCTCTACAACGAACTCAATCTTGGTCGGAGAG
AGGAGTACGACGTGCTGGACAAGCGGAGAGGACGGGACCCAGAAATGGGC
GGGAAGCCGCGCAGAAAGAATCCCCAAGAGGGCCTGTACAACGAGCTCCA
AAAGGATAAGATGGCAGAAGCCTATAGCGAGATTGGTATGAAAGGGGAAC
GCAGAAGAGGCAAAGGCCACGACGGACTGTACCAGGGACTCAGCACCGCC
ACCAAGGACACCTATGACGCTCTTCACATGCAGGCCCTGCCGCCTCGG,

optionally wherein any codon in SEQ ID NO: 14 may each independently be replaced by another codon encoding the same amino acid, preferably wherein the nucleic acid sequence is thereby codon optimised for expression in cells of a desired species, such as in human cells, according to codon optimisation principles known in the art, such as wherein the CAR is encoded by a nucleic acid comprising, consisting essentially of or consisting of a nucleic acid sequence at least 60%, at least 70%, at least 80%, at least 90%, or at least 95% identical to SEQ ID NO: 14.

To allow for expression of a nucleic acid encoding a CAR, that is for the production of the CAR protein in a suitable system, such as a host cell, driven by the genetic information contained in the nucleic acid, the nucleic acid may be provided in an expressible format, such as inserted or otherwise made a component of an expression cassette or vector, as is common in the art. Accordingly, aspects of the invention also relate to a nucleic acid encoding the CAR molecule as disclosed herein in an expressible format. Certain embodiments relate to an expression cassette or expression vector comprising the CAR-encoding nucleic acids.

The term “expression cassette” encompasses a nucleic acid molecule, typically DNA, into which a coding sequence for a protein or proteins of interest may be inserted to be expressed, wherein said nucleic acid molecule comprises one or more nucleic acid sequences operably linked to and controlling the expression of the coding sequence (regulatory sequences), non-limiting examples of which include promoter sequences and transcription terminators. An “operable linkage” is a linkage in which regulatory sequences and sequences sought to be expressed are connected in such a way as to permit said expression. For example, sequences, such as, e.g., a promoter and a coding sequence for a protein of interest, may be said to be operably linked if the nature of the linkage between said sequences does not: (1) result in the introduction of a frame-shift mutation, (2) interfere with the ability of the promoter to direct the transcription of the coding sequence, (3) interfere with the ability of the coding sequence to be transcribed from the promoter sequence. Hence, “operably linked” may mean incorporated into a genetic construct so that expression control sequences, such as a promoter, effectively control transcription/expression of a sequence of interest. The precise nature of transcriptional and translational regulatory sequences or elements required for expression may vary between expression environments, but typically transcriptional regulatory sequences include a promoter, optionally an enhancer, and a transcription terminator.

Reference to a “promoter” is to be taken in its broadest context and includes transcriptional regulatory sequences required for accurate transcription initiation and where applicable accurate spatial and/or temporal control of gene expression or its response to, e.g., internal or external (e.g., exogenous) stimuli. More particularly, “promoter” may depict a region on a nucleic acid molecule, preferably DNA molecule, to which an RNA polymerase binds and initiates transcription. A promoter is preferably, but not necessarily, positioned upstream, i.e., 5′, of the sequence the transcription of which it controls. Typically, in prokaryotes a promoter region may contain both the promoter per se and sequences which, when transcribed into RNA, will signal the initiation of protein synthesis (e.g., Shine-Dalgarno sequence). A promoter sequence can also include “enhancer regions”, which are one or more regions of DNA that can be bound with proteins (namely the transacting factors) to enhance transcription levels of genes in a gene-cluster. The enhancer, while typically at the 5′ end of a coding region, can also be separate from a promoter sequence, e.g., can be within an intronic region of a gene or 3′ to the coding region of the gene.

In certain embodiments, promoters may be constitutive or inducible. A constitutive promoter is understood to be a promoter whose expression is constant under standard culturing conditions. Inducible promoters are promoters that are responsive to one or more induction cues. For example, an inducible promoter can be chemically regulated (e.g., a promoter whose transcriptional activity is regulated by the presence or absence of a chemical inducing agent such as an alcohol, tetracycline, a steroid, a metal, or other small molecule) or physically regulated (e.g., a promoter whose transcriptional activity is regulated by the presence or absence of a physical inducer such as light or high or low temperatures). An inducible promoter can also be indirectly regulated by one or more transcription factors that are themselves directly regulated by chemical or physical cues. Non-limiting examples of promoters include T7, U6, H1, retroviral Rous sarcoma virus (RSV) LTR promoter, the cytomegalovirus (CMV) promoter, the SV40 promoter, the dihydrofolate reductase promoter, the β-actin promoter, the phosphoglycerol kinase (PGK) promoter, and the EF1α promoter.

The terms “terminator” or “transcription terminator” refer generally to a sequence element at the end of a transcriptional unit which signals termination of transcription. For example, a terminator is usually positioned downstream of, i.e., 3′ of a coding sequence encoding a polypeptide of interest. For instance, where a recombinant nucleic acid contains two or more coding sequences, e.g., successively ordered and forming together a multicistronic transcription unit, a transcription terminator may be advantageously positioned 3′ to the most downstream coding sequence.

The terms “expression vector” or “vector” as used herein refer to nucleic acid molecules, typically DNA, to which nucleic acid fragments may be inserted and cloned, i.e., propagated. Hence, a vector will typically contain one or more unique restriction sites, and may be capable of autonomous replication in a defined cell or vehicle organism such that the cloned sequence is reproducible. A vector may also preferably contain a selection marker, such as, e.g., an antibiotic resistance gene, to allow selection of recipient cells that contain the vector. Vectors may include, without limitation, plasmids, phagemids, bacteriophages, bacteriophage-derived vectors, PAC, BAC, linear nucleic acids, e.g., linear DNA, transposons, viral vectors, etc., as appropriate (see, e.g., Sambrook et al., 1989; Ausubel 1992). Viral vectors may include inter alia retroviral vectors, lentiviral vectors, adenoviral vectors, or adeno-associated viral vectors, for example, vectors based on HIV, SV40, EBV, HSV or BPV. Expression vectors are generally configured to allow for and/or effect the expression of nucleic acids or open reading frames introduced thereto in a desired expression system, e.g., in vitro, in a cell, organ and/or organism. For example, expression vectors may advantageously comprise suitable regulatory sequences.

Factors of importance in selecting a particular vector include inter alio: choice of recipient cell, ease with which recipient cells that contain the vector may be recognised and selected from those recipient cells which do not contain the vector; the number of copies of the vector which are desired in particular recipient cells; whether it is desired for the vector to integrate into the chromosome or to remain extra-chromosomal in the recipient cells; and whether it is desirable to be able to “shuttle” the vector between recipient cells of different species.

Expression vectors can be autonomous or integrative. A nucleic acid can be in introduced into a cell in the form of an expression vector such as a plasmid, phage, transposon, cosmid or virus particle. The recombinant nucleic acid can be maintained extrachromosomally or it can be integrated into the cell chromosomal DNA. Expression vectors can contain selection marker genes encoding proteins required for cell viability under selected conditions (e.g., URA3, which encodes an enzyme necessary for uracil biosynthesis, or LEU2, which encodes an enzyme required for leucine biosynthesis, or TRP1, which encodes an enzyme required for tryptophan biosynthesis) to permit detection and/or selection of those cells transformed with the desired nucleic acids. Expression vectors can also include an autonomous replication sequence (ARS). The ARS may comprise a centromere (CEN) and an origin of replication (ORD. For example, the ARS may be ARS18 or ARS68.

Integrative vectors generally include a serially arranged sequence of at least a first insertable DNA fragment, a selectable marker gene, and a second insertable DNA fragment. The first and second insertable DNA fragments are each about 200 (e.g., about 250, about 300, about 350, about 400, about 450, about 500, or about 1000 or more) nucleotides in length and have nucleotide sequences which are homologous to portions of the genomic DNA of the cell species to be transformed. A nucleotide sequence containing a nucleic acid of interest for expression is inserted in this vector between the first and second insertable DNA fragments, whether before or after the marker gene. Integrative vectors can be linearized prior to transformation to facilitate the integration of the nucleotide sequence of interest into the cell genome.

Prior to introducing the vectors into a cell of interest, the vectors can be grown (e.g., amplified) in bacterial cells such as Escherichia coli (E. coli). The vector DNA can be isolated from bacterial cells by any of the methods known in the art, which result in the purification of vector DNA from the bacterial milieu. The purified vector DNA can be extracted extensively with phenol, chloroform, and ether, to ensure that no E. coli proteins are present in the plasmid DNA preparation, since these proteins can be toxic to mammalian cells.

In certain embodiments, the nucleic acid encoding the CAR in an expressible format may be a DNA molecule configured to drive the expression of the CAR protein (transcription of the DNA CAR coding sequence into corresponding CAR messenger RNA molecule and translation of the latter into a protein by cellular translation machinery) when the DNA molecule is introduced into an animal cell, such as a mammalian cell, a human cell, such as in particular a human NK cell. In certain embodiments, the nucleic acid encoding the CAR in an expressible format may be an expression cassette or expression vector comprising a coding sequence for the CAR protein. In certain embodiments, this may concern a DNA expression cassette or expression vector comprising a DNA coding sequence for the CAR protein. In certain other embodiments, the nucleic acid encoding the CAR in an expressible format may be an RNA molecule, such as a messenger RNA molecule (mRNA), configured to drive the expression of the CAR protein (translation of the coding sequence comprised by the RNA molecule into a protein by cellular translation machinery) when the mRNA molecule is introduced into an animal cell, such as a mammalian cell, a human cell, such as in particular a human NK cell. Such mRNA may be produced by any suitable means available in the art, such as by in vitro transcription from an expression cassette or vector comprising the CAR coding sequence.

There exist various well-known methods of introducing nucleic acids into animal cells, any of which may be used herein. At the simplest, the nucleic acid can be directly injected into the target cell. Other methods include fusion of the recipient cell with bacterial protoplasts containing the nucleic acid, the use of compositions like calcium chloride, rubidium chloride, lithium chloride, calcium phosphate, DEAE dextran, cationic lipids or liposomes or methods like receptor-mediated endocytosis, biolistic particle bombardment (“gene gun” method), infection with viral vectors (i.e. derived from lentivirus, adeno-associated virus (AAV), adenovirus, retrovirus or antiviruses), electroporation, and the like. Other techniques or methods which are suitable for delivering nucleic acid molecules to target cells include the continuous delivery of an NA molecule from poly (lactic-Co-Glycolic Acid) polymeric microspheres or the direct injection of protected (stabilized) NA molecule(s) into micropumps delivering the product. Another possibility is the use of implantable drug-releasing biodegradable microspheres. Also envisaged is encapsulation of NA in various types of liposomes (immunoliposomes, PEGylated (immuno) liposomes), cationic lipids and polymers, nanoparticles or dendrimers, poly (lactic-Co-Glycolic Acid) polymeric microspheres, implantable drug-releasing biodegradable microspheres, etc.; and co-injection of NA with protective agent like the nuclease inhibitor aurintricarboxylic acid. It shall be clear that also a combination of different above-mentioned delivery modes or methods may be used.

Further to the above illustration and guidance, the phrase “a cell engineered to express” a protein of interest may thus in particular denote a cell altered or manipulated by man to comprise a nucleic acid encoding the protein of interest, such as a cell into which an exogenous nucleic acid encoding the protein of interest has been introduced or inserted by available technical means.

As already discussed, the present invention in particular envisages genetic engineering of natural killer (NK) cells to express the presently described CAR proteins. The expression level of the CAR, that is the quantity of the CAR protein produced by the NK cell, may vary within an acceptable range which may typically be determined by, on the lower end, the preference or necessity that the expression level is sufficient to endow the cell with specificity for CD70 on target cells coupled with meaningful intensity of intracellular signal generation within the NK cell such as to elicit an action on the CD70 positive target cell, such as in particular a cytotoxic action (e.g., damaging or killing the CD70 positive target cell) and, on the higher end, the preference or necessity to avoid reduction in the viability or functionality of the NK cell due to excessive overexpression of the exogenous CAR protein. Such quantitative considerations can be applied by the skilled person.

Whereas the qualifier “isolated” need not be expressly recited with relation to NK cells as intended herein, it may conveniently be included, as the present disclosure pertains to manipulation of NK cells outside of the body, in vitro or ex vivo, and subsequent therapeutic use of so-manipulated NK cells. The term “isolated” with reference to a particular component generally denotes that such component exists in separation from — for example, has been separated from or prepared and/or maintained in separation from — one or more other components of its natural environment. More particularly, the term “isolated” as used herein in relation to a cell or cell population denotes that such cell or cell population does not form part of an animal or human body, for example, the cell may be cultured, sorted or stored in vitro or ex vivo.

Native NK cells represent a distinct population of lymphocytes in terms of both phenotype and function. NK cells have a large granular lymphocyte morphology and express characteristic NK cell surface receptors, and lack both T cell receptor (TCR) rearrangement and T cell, B cell, monocyte and/or macrophage cell surface markers. The cells kill by releasing small cytoplasmic granules of proteins (perforin and granzyme) that cause the target cell to die by apoptosis. NK cells possess mechanisms distinguishing between potential “target” cells and healthy cells via a multitude of inhibitory and activating receptors that engage MHC class I molecules, MHC class I-like molecules, and molecules unrelated to MHC. Inhibitory NK cell receptors include HLA-E (CD94/NKG2A); HLA-C (group 1 or 2), KIR2DL; KIR3DL (HLA-B Bw4) and HLA-A3 or A4 + peptide. Activating NK cell receptors include HLA-E (CD94/NKG2C); KIR2DS (HLA-C) and KIR3DS (HLA-Bw4). Other receptors include the NK cell receptor protein-1 and the low affinity receptor for the Fc portion of IgG (FcyRIII; CD 16). “Activating” and “inhibitory” surface receptors regulate the native NK cell's cytotoxic activity.

NK cells can be detected by specific surface markers, such as CD16, CD56, and CD8 in humans with absence of CD3 expression.

In certain embodiments, NK cells may be isolated from a subject, such as a human subject. In certain embodiments, NK cells may be isolated from cord blood or peripheral blood of a subject. In certain embodiments, NK cells may be in vitro differentiated from a hematopoietic stem cell or from an induced pluripotent stem (iPS) cell. In certain embodiments, NK cells may be derived from human peripheral blood mononuclear cells (PBMC), unstimulated leukapheresis products (PBSC), human embryonic stem cells (hESCs), induced pluripotent stem cells (iPSCs), bone marrow, or umbilical cord blood. Methods to isolate or derive NK cells from such sources are well known in the art. In particular embodiments, NK cells may be isolated from cord blood (CB), peripheral blood (PB), bone marrow, or stem cells. In particular embodiments, NK cells may be isolated from pooled CB. The CB may be pooled from 2, 3, 4, 5, 6, 7, 8, 10, 20 or more units.

In certain embodiments, a starting population of NK cells may be obtained by isolating mononuclear cells using Ficoll density gradient centrifugation. The cell culture may be depleted of any cells expressing CD3, CD14, and/or CD19 cells and may be characterised to determine the percentage of CD56+/CD3− cells (NK cells).

In certain embodiments, the NK cells may be autologous or allogeneic relative to a subject to whom they are to be administered. In certain preferred embodiments, the NK cells may be allogeneic relative to a subject to whom they are to be administered, as NK cells can be applied in allogeneic settings without promoting graft-versus-host disease. In certain embodiments, the NK cells may be haplotype matched for the subject to whom they are to be administered.

In certain embodiments, NK cells may be from a clonal NK-cell line. NK cell lines are typically derived from NK lymphomas/leukemias as known in the art. Non-limiting examples of NK cell lines include YTS, NK92 (NK-92), NK3.3, NKL, and NK101 cell lines. In certain preferred embodiments, the NK cell is an NK-92 cell. NK-92 cells are well-characterised and frequently used in pre-clinical and clinical settings.

Primary NK cells can be isolated from patients, isolated from healthy donors, or purchased from public cell collections. By means of an example and without limitation, primary human CD56+ NK cells isolated from blood are available from American Type Culture Collection (ATCC), 10801 University Boulevard, Manassas, VA 20110, USA, under catalogue number PCS-800-019, and NK cell lines, in particular NK-92 cell lines, are available from ATCC under catalogue numbers CRL-2407 and CRL-2408 (the latter is derived from CRL-2407 and is interleukin-2 independent), as well as from the German Collection of Microorganisms and Cell Cultures GmbH (DSMZ) under catalogue numbers # ACC 488.

In certain embodiments, the NK cells may be cultured in vitro. Ways to culture, propagate and expand NK cells and modulate their properties in vitro, including ex vivo, are well characterised. Without limitation, the Examples section demonstrates culturing NK cells in standard α-MEM medium supplemented with suitable serum or sera (e.g., fetal bovine serum and/or horse serum), antibiotics mix, L-glutamine and interleukin 2 (IL-2). Further manners to culture NK cells, such as using cytokine interleukins including IL-2, 12, 15, 18 and/or 21, cocultivation with autologous accessory non-NK cells, or addition of growth-inactivated feeder cells, are described for example in Granzin et al. (Front Immunol. 2017, 8:458).

In certain embodiments, the NK cell as intended herein may be engineered to further express one or more immunostimulatory cytokine, preferably one or more immunostimulatory interleukin (IL), in particular one or more human cytokines or interleukins. Examples of suitable interleukins include IL-15, IL-12, and/or IL-21, in particular human IL-15, IL-12, and/or IL-21. Preferably, the immunostimulatory interleukin is IL-15, or IL-21, or both. Preferably, the immunostimulatory interleukin is human IL-15, or human IL-21, or both. Particularly preferably, the immunostimulatory interleukin is human IL-15. In certain embodiments, the expression of the CAR and of the optional one or more immunostimulatory cytokine is each independently constitutive or inducible.

By means of guidance and without limitation, human interleukin-15 isoform 1 preproprotein and human interleukin-15 isoform 2 preproprotein are annotated under Genbank accession no: NP_000576.1 and NP_751915.1, respectively.

One particular example of human IL-15 isoform 1 preprotein is reproduced below:

(SEQ ID NO: 15)
MRISKPHLRSISIQCYLCLLLNSHFLTEAGIHVFILGCFSAGLPKTEANW
VNVISDLKKIEDLIQSMHIDATLYTESDVHPSCKVTAMKCFLLELQVISL
ESGDASIHDTVENLIILANNSLSSNGNVTESGCKECEELEEKNIKEFLQS
FVHIVQMFINTS.

In certain embodiments, the IL-15 denotes a polypeptide comprising, consisting essentially of or consisting of an amino acid sequence at least 80% identical to SEQ ID NO: 15, such as at least 85% identical to SEQ ID NO: 15, preferably at least 90% identical to SEQ ID NO: 15, such as at least 95% identical to SEQ ID NO: 15, more preferably at least 96%, at least 97%, at least 98%, or at least 99% identical to SEQ ID NO: 15. In particularly preferred embodiments, the IL-15 comprises, consists essentially of or consists of the amino acid sequence set forth in SEQ ID NO: 15.

In certain embodiments, the human IL-15, such as in particular the one set forth in SEQ ID NO: 15, may be encoded by a nucleic acid comprising, consisting essentially of or consisting of the nucleic acid sequence set forth in SEQ ID NO: 16:

(SEQ ID NO: 16)
ATGAGAATCAGCAAGCCCCACCTGAGATCCATCAGCATCCAGTGCTACCT
GTGCCTGCTGCTGAACAGCCACTTTCTGACAGAGGCCGGCATCCACGTGT
TCATCCTGGGCTGTTTTTCTGCCGGCCTGCCTAAGACCGAGGCCAACTGG
GTTAACGTGATCAGCGACCTGAAGAAGATCGAGGACCTGATCCAGAGCAT
GCACATCGACGCCACACTGTACACCGAGAGCGACGTGCACCCTAGCTGTA
AAGTGACCGCCATGAAGTGCTTTCTGCTGGAACTGCAAGTGATCAGCCTG
GAAAGCGGCGACGCCAGCATCCACGACACCGTGGAAAACCTGATCATCCT
GGCCAACAACAGCCTGAGCAGCAACGGCAATGTGACCGAGTCCGGCTGCA
AAGAGTGCGAGGAACTGGAAGAGAAGAATATCAAAGAGTTCCTGCAGAGC
TTCGTGCACATCGTGCAGATGTTCATCAACACCAGC,

optionally wherein any codon in SEQ ID NO: 16 may each independently be replaced by another codon encoding the same amino acid, preferably wherein the nucleic acid sequence is thereby codon optimised for expression in cells of a desired species, such as in human cells, according to codon optimisation principles known in the art, such as wherein the human IL-15 is encoded by a nucleic acid comprising, consisting essentially of or consisting of a nucleic acid sequence at least 60%, at least 70%, at least 80%, at least 90%, or at least 95% identical to SEQ ID NO: 16.

By means of guidance and without limitation, human interleukin-21 isoform 1 precursor and human IL-21 isoform 2 precursor are annotated under Genbank accession no: NP_068575.1 and NP_001193935.1, respectively.

One particular example of human IL-21 isoform 1 precursor (NP_068575.1) is reproduced below:

(SEQ ID NO: 23)
MRSSPGNMERIVICLMVIFLGTLVHKSSSQGQDRHMIRMRQLIDIVDQLK
NYVNDLVPEFLPAPEDVETNCEWSAFSCFQKAQLKSANTGNNERIINVSI
KKLKRKPPSTNAGRRQKHRLTCPSCDSYEKKPPKEFLERFKSLLQKMIHQ
HLSSRTHGSEDS.

One particular example of human IL-21 isoform 2 precursor (NP_001193935.1) is reproduced below:

(SEQ ID NO: 24)
MRSSPGNMERIVICLMVIFLGTLVHKSSSQGQDRHMIRMRQLIDIVDQLK
NYVNDLVPEFLPAPEDVETNCEWSAFSCFQKAQLKSANTGNNERIINVSI
KKLKRKPPSTNAGRRQKHRLTCPSCDSYEKKPPKEFLERFKSLLQKVSTL
SFI.

In certain embodiments, the IL-21 denotes a polypeptide comprising, consisting essentially of or consisting of an amino acid sequence at least 80% identical to SEQ ID NO: 23 or 24, preferably SEQ ID NO: 23; such as at least 85% identical to SEQ ID NO: 23 or 24, preferably SEQ ID NO: 23; preferably at least 90% identical to SEQ ID NO: 23 or 24, preferably SEQ ID NO: 23; such as at least 95% identical to SEQ ID NO: 23 or 24, preferably SEQ ID NO: 23; more preferably at least 96%, at least 97%, at least 98%, or at least 99% identical to SEQ ID NO: 23 or 24, preferably SEQ ID NO: 23. In particularly preferred embodiments, the IL-21 comprises, consists essentially of or consists of the amino acid sequence set forth in SEQ ID NO: 23 or 24, preferably SEQ ID NO: 23.

In certain embodiments, the human IL-21, such as in particular the one set forth in SEQ ID NO: 23 or 24, may be encoded by a nucleic acid comprising, consisting essentially of or consisting of the nucleic acid sequence as annotated in GenBank under accession numbers NM_021803.4 or NM_001207006.2, respectively, or as set forth in SEQ ID NO: 25:

(SEQ ID NO: 25)
ATGAGATCCAGTCCTGGCAACATGGAGAGGATTGTCATCTGTCTGATGGT
CATCTTCTTGGGGACACTGGTCCACAAATCAAGCTCCCAAGGTCAAGATC
GCCACATGATTAGAATGCGTCAACTTATAGATATTGTTGATCAGCTGAAA
AATTATGTGAATGACTTGGTCCCTGAATTTCTGCCAGCTCCAGAAGATGT
AGAGACAAACTGTGAGTGGTCAGCTTTTTCCTGCTTTCAGAAGGCCCAAC
TAAAGTCAGCAAATACAGGAAACAATGAAAGGATAATCAATGTATCAATT
AAAAAGCTGAAGAGGAAACCACCTTCCACAAATGCAGGGAGAAGACAGAA
ACACAGACTAACATGCCCTTCATGTGATTCTTATGAGAAAAAACCACCCA
AAGAATTCCTAGAAAGATTCAAATCACTTCTCCAAAAGATGATTCATCAG
CATCTGTCCTCTAGAACACACGGAAGTGAAGATTCC,

optionally wherein any codon in NM_021803.4, NM_001207006.2 or in SEQ ID NO: 25 may each independently be replaced by another codon encoding the same amino acid, preferably wherein the nucleic acid sequence is thereby codon optimised for expression in cells of a desired species, such as in human cells, according to codon optimisation principles known in the art, such as wherein the human IL-21 is encoded by a nucleic acid comprising, consisting essentially of or consisting of a nucleic acid sequence at least 60%, at least 70%, at least 80%, at least 90%, or at least 95% identical to NM_021803.4, NM_001207006.2 or SEQ ID NO: 25.

By means of guidance and without limitation, human interleukin-12 subunit alpha (IL-12A) precursor is annotated under Genbank accession no: NP_000873.2 (isoform 1), NP_001341511.1 (isoform 2), NP_001341512.1 (isoform 3), and NP_001384921.1 (isoform 4). By means of guidance and without limitation, human interleukin-12 subunit beta (IL-12B) precursor is annotated under Genbank accession no: NP_002178. Together, IL-12A (p35) and IL-12B (p40) form the heterodimeric active cytokine referred to as p70.

One particular example of human IL-12A, namely isoform 1 precursor (NP_000873.2), is reproduced below:

(SEQ ID NO: 26)
MWPPGSASQPPPSPAAATGLHPAARPVSLQCRLSMCPARSLLLVATLVLL
DHLSLARNLPVATPDPGMFPCLHHSQNLLRAVSNMLQKARQTLEFYPCTS
EEIDHEDITKDKTSTVEACLPLELTKNESCLNSRETSFITNGSCLASRKT
SFMMALCLSSIYEDLKMYQVEFKTMNAKLLMDPKRQIFLDQNMLAVIDEL
MQALNFNSETVPQKSSLEEPDFYKTKIKLCILLHAFRIRAVTIDRVMSYL
NAS.

One particular example of human IL-12A subunit to be co-expressed with the CAR is reproduced below:

(SEQ ID NO: 27)
MCPARSLLLVATLVLLDHLSLARNLPVATPDPGMFPCLHHSQNLLRAVSN
MLQKARQTLEFYPCTSEEIDHEDITKDKTSTVEACLPLELTKNESCLNSR
ETSFITNGSCLASRKTSFMMALCLSSIYEDLKMYQVEFKTMNAKLLMDPK
RQIFLDQNMLAVIDELMQALNFNSETVPQKSSLEEPDFYKTKIKLCILLH
AFRIRAVTIDRVMSYLNAS.

One particular example of human IL-12B precursor (NP_002178.2) is reproduced below:

(SEQ ID NO: 28)
MCHQQLVISWFSLVFLASPLVAIWELKKDVYVVELDWYPDAPGEMVVLTC
DTPEEDGITWTLDQSSEVLGSGKTLTIQVKEFGDAGQYTCHKGGEVLSHS
LLLLHKKEDGIWSTDILKDQKEPKNKTFLRCEAKNYSGRFTCWWLTTIST
DLTFSVKSSRGSSDPQGVTCGAATLSAERVRGDNKEYEYSVECQEDSACP
AAEESLPIEVMVDAVHKLKYENYTSSFFIRDIIKPDPPKNLQLKPLKNSR
QVEVSWEYPDTWSTPHSYFSLTFCVQVQGKSKREKKDRVFTDKTSATVIC
RKNASISVRAQDRYYSSSWSEWASVPCS.

In certain embodiments, IL-12A and IL-12B denote the respective polypeptides comprising, consisting essentially of or consisting of an amino acid sequence at least 80% identical to SEQ ID NO: 26-28, such as at least 85% identical to SEQ ID NO: 26-28, preferably at least 90% identical to SEQ ID NO: 26-28, such as at least 95% identical to SEQ ID NO: 26-28, more preferably at least 96%, at least 97%, at least 98%, or at least 99% identical to SEQ ID NO: 26-28. In particularly preferred embodiments, IL-12A comprises, consists essentially of or consists of the amino acid sequence set forth in SEQ ID NO: 26 or 27. In particularly preferred embodiments, IL-12B comprises, consists essentially of or consists of the amino acid sequence set forth in SEQ ID NO: 28.

In certain embodiments, the human IL-12A precursor may be encoded by a nucleic acid comprising, consisting essentially of or consisting of the nucleic acid sequence as annotated in GenBank under accession number NM_000882.3 (isoform 1), NM_001354582.2 (isoform 2), NM_001354583.2 (isoform 3), NM_001397992.1 (isoform 4), or as set forth in SEQ ID NO: 29:

(SEQ ID NO: 29)
ATGTGTCCAGCGCGCAGCCTCCTCCTTGTGGCTACCCTGGTCCTCCTGGA
CCACCTCAGTTTGGCCAGAAACCTCCCCGTGGCCACTCCAGACCCAGGAA
TGTTCCCATGCCTTCACCACTCCCAAAACCTGCTGAGGGCCGTCAGCAAC
ATGCTCCAGAAGGCCAGACAAACTCTAGAATTTTACCCTTGCACTTCTGA
AGAGATTGATCATGAAGATATCACAAAAGATAAAACCAGCACAGTGGAGG
CCTGTTTACCATTGGAATTAACCAAGAATGAGAGTTGCCTAAATTCCAGA
GAGACCTCTTTCATAACTAATGGGAGTTGCCTGGCCTCCAGAAAGACCTC
TTTTATGATGGCCCTGTGCCTTAGTAGTATTTATGAAGACTTGAAGATGT
ACCAGGTGGAGTTCAAGACCATGAATGCAAAGCTTCTGATGGATCCTAAG
AGGCAGATCTTTCTAGATCAAAACATGCTGGCAGTTATTGATGAGCTGAT
GCAGGCCCTGAATTTCAACAGTGAGACTGTGCCACAAAAATCCTCCCTTG
AAGAACCGGATTTTTATAAAACTAAAATCAAGCTCTGCATACTTCTTCAT
GCTTTCAGAATTCGGGCAGTGACTATTGATAGAGTGATGAGCTATCTGAA
TGCTTCC,

optionally wherein any codon in NM_000882.3, NM_001354582.2, NM_001354583.2, NM_001397992.1, or in SEQ ID NO: 29 may each independently be replaced by another codon encoding the same amino acid, preferably wherein the nucleic acid sequence is thereby codon optimised for expression in cells of a desired species, such as in human cells, according to codon optimisation principles known in the art, such as wherein the human IL-12A is encoded by a nucleic acid comprising, consisting essentially of or consisting of a nucleic acid sequence at least 60%, at least 70%, at least 80%, at least 90%, or at least 95% identical to NM_000882.3, NM_001354582.2, NM_001354583.2, NM_001397992.1, or SEQ ID NO: 29.

In certain embodiments, the human IL-12B precursor may be encoded by a nucleic acid comprising, consisting essentially of or consisting of the nucleic acid sequence as annotated in GenBank under accession number NM_002187.2 or as set forth in SEQ ID NO: 30:

(SEQ ID NO: 30)
ATGTGTCACCAGCAGTTGGTCATCTCTTGGTTTTCCCTGGTTTTTCTGGC
ATCTCCCCTCGTGGCCATATGGGAACTGAAGAAAGATGTTTATGTCGTAG
AATTGGATTGGTATCCGGATGCCCCTGGAGAAATGGTGGTCCTCACCTGT
GACACCCCTGAAGAAGATGGTATCACCTGGACCTTGGACCAGAGCAGTGA
GGTCTTAGGCTCTGGCAAAACCCTGACCATCCAAGTCAAAGAGTTTGGAG
ATGCTGGCCAGTACACCTGTCACAAAGGAGGCGAGGTTCTAAGCCATTCG
CTCCTGCTGCTTCACAAAAAGGAAGATGGAATTTGGTCCACTGATATTTT
AAAGGACCAGAAAGAACCCAAAAATAAGACCTTTCTAAGATGCGAGGCCA
AGAATTATTCTGGACGTTTCACCTGCTGGTGGCTGACGACAATCAGTACT
GATTTGACATTCAGTGTCAAAAGCAGCAGAGGCTCTTCTGACCCCCAAGG
GGTGACGTGCGGAGCTGCTACACTCTCTGCAGAGAGAGTCAGAGGGGACA
ACAAGGAGTATGAGTACTCAGTGGAGTGCCAGGAGGACAGTGCCTGCCCA
GCTGCTGAGGAGAGTCTGCCCATTGAGGTCATGGTGGATGCCGTTCACAA
GCTCAAGTATGAAAACTACACCAGCAGCTTCTTCATCAGGGACATCATCA
AACCTGACCCACCCAAGAACTTGCAGCTGAAGCCATTAAAGAATTCTCGG
CAGGTGGAGGTCAGCTGGGAGTACCCTGACACCTGGAGTACTCCACATTC
CTACTTCTCCCTGACATTCTGCGTTCAGGTCCAGGGCAAGAGCAAGAGAG
AAAAGAAAGATAGAGTCTTCACGGACAAGACCTCAGCCACGGTCATCTGC
CGCAAAAATGCCAGCATTAGCGTGCGGGCCCAGGACCGCTACTATAGCTC
ATCTTGGAGCGAATGGGCATCTGTGCCCTGCAGT,

optionally wherein any codon in NM_002187.2 or in SEQ ID NO: 30 may each independently be replaced by another codon encoding the same amino acid, preferably wherein the nucleic acid sequence is thereby codon optimised for expression in cells of a desired species, such as in human cells, according to codon optimisation principles known in the art, such as wherein the human IL-12B is encoded by a nucleic acid comprising, consisting essentially of or consisting of a nucleic acid sequence at least 60%, at least 70%, at least 80%, at least 90%, or at least 95% identical to NM_002187.2 or SEQ ID NO: 30.

In certain embodiments, the CAR and the immunostimulatory cytokine such as IL-15, IL-12, and/or IL-21, such as in particular IL-15 and/or IL-21, such as even more particularly IL-15, may be encoded by two separate open reading frames (ORF). In certain embodiments, the two separate ORFs may be comprised by the same polynucleotide or by two separate polynucleotides. In certain embodiments, the two separate ORFs may be comprised by a single transcription unit or by two separate transcription units. In certain embodiments, the two separate ORFs may be comprised by a single mRNA molecule or by two separate mRNA molecules.

In certain embodiments, the CAR and the immunostimulatory cytokine such as IL-15, IL-12, and/or IL-21, such as in particular IL-15 and/or IL-21, such as even more particularly IL-15, may be comprised by a single ORF, with a peptide sequence susceptible to ribosomal skipping or to spontaneous proteolysis arranged in between the CAR and the cytokine amino acid sequences. By means of an example, a sequence encoding a 2A self-cleaving peptide may be interposed between the sequence encoding the CAR and the sequence encoding the cytokine. Examples of 2A peptides include T2A (EGRGSLLTCGDVEENPGP, SEQ ID NO: 17), P2A (ATNFSLLKQAGDVEENPGP, SEQ ID NO: 18), E2A (QCTNYALLKLAGDVESNPGP, SEQ ID NO: 19), and F2A (VKQTLNFDLLKLAGDVESNPGP, SEQ ID NO: 20). An optional GSG tripeptide may be included at the N-terminus to increase efficiency. Accordingly, in certain embodiments, the CAR and the cytokine are encoded within the same ORF with a sequence encoding a polypeptide comprising a 2A peptide interposed therebetween.

Accordingly, by means of an illustration, in certain particularly preferred embodiments, a CAR-P2A-IL15 precursor comprises, consists essentially of, or consists of the amino acid as set forth in SEQ ID NO: 21 below, with the signal sequence in bold font, the extracellular and intramembrane domains of CD27 in standard font, the 4-1BB intracellular co-stimulatory domain in italics, the CD3ζ intracellular activation domain underlined, the GSG-P2A self-cleaving peptide in bold italics, and the IL-15 bold underlined:

(SEQ ID NO: 21)
MARPHPWWLCVLGTLVGLSATPAPKSCPERHYWAQGKLCCQMCEPGTFLVKDCDQHR
KAAQCDPCIPGVSFSPDHHTRPHCESCRHCNSGLLVRNCTITANAECACRNGWQCRDKEC
TECDPLPNPSLTARSSQALSPHPQPTHLPYVSEMLEARTAGHMQTLADFRQLPARTLSTHW
PPQRSLCSSDFIRILVIFSGMFLVFTLAGALFLHKRGRKKLLYIFKQPFMRPVQTTQEEDGCSC
RFPEEEEGGCELRVKFSRSADAPAYKQGQNQLYNELNLGRREEYDVLDKRRGRDPEMGG
KPRRKNPQEGLYNELQKDKMAEAYSEIGMKGERRRGKGHDGLYQGLSTATKDTYDALH
MQALPPREF [symbol] [symbol] MRISKPHLRSISIQCYLCLLLNSHFLTEA
GIHVFILGCFSAGLPKTEANWVNVISDLKKIEDLIQSMHIDATLYTESDVHPSCKVTA
MKCFLLELQVISLESGDASIHDTVENLIILANNSLSSNGNVTESGCKECEELEEKNIKEF
LQSFVHIVQMFINTSEF.

In certain embodiments the CAR-P2A-IL15 precursor comprises, consists essentially of or consists of an amino acid sequence at least 80% identical, preferably at least 85% identical, more preferably at least 90% identical, even more preferably at least 95% identical, such as particularly preferably at least 96%, at least 97%, at least 98% or at least 99% or 100% identical to SEQ ID NO: 21.

In certain embodiments, the CAR-P2A-IL15 precursor form as taught herein, such as in particular the one set forth in SEQ ID NO: 21, may be encoded by a nucleic acid comprising, consisting essentially of or consisting of the nucleic acid sequence set forth in SEQ ID NO: 22 or 31, with the ATG start and TAA stop codons of the CAR-P2A-IL15 coding sequence in bold, an SpeI restriction site underlined, GCCACC Kozak sequence in italics, and an XhoI restriction site double underlined, and the EcoRI restriction site flanking the IL-15 cassette in bold italics (evidently, other restriction sites may be included, or none, depending on experimental convenience):

(SEQ ID NO: 22)
ACTAGTGCCACCATGGCCAGACCTCATCCTTGGTGGCTGTGTGTGCTGGGCACACTCGT
TGGCCTGTCTGCTACACCAGCTCCTAAGAGCTGCCCCGAGAGACACTATTGGGCCCAG
GGCAAGCTGTGCTGCCAGATGTGTGAACCTGGCACCTTCCTGGTCAAGGACTGCGACC
AGCACAGAAAGGCCGCTCAGTGCGATCCTTGTATCCCCGGCGTGTCCTTCTCTCCCGAC
CACCACACAAGACCTCACTGCGAGAGCTGCAGACACTGCAATTCTGGACTGCTCGTGC
GGAACTGCACCATCACAGCCAATGCCGAGTGCGCCTGCAGAAATGGCTGGCAGTGCC
GGGACAAAGAATGTACCGAGTGCGACCCTCTGCCTAATCCTAGCCTGACCGCCAGAAG
CAGCCAGGCTTTGTCTCCTCATCCTCAGCCTACACATCTGCCCTACGTGTCCGAGATGC
TGGAAGCCAGAACAGCCGGCCATATGCAGACCCTGGCCGACTTTAGACAGCTGCCCGC
CAGAACACTGAGCACCCATTGGCCTCCACAGAGAAGCCTGTGCAGCAGCGACTTCATC
CGGATCCTCGTGATCTTCAGCGGCATGTTCCTGGTGTTCACACTGGCTGGCGCCCTGTT
TCTGCATAAGCGCGGTCGGAAGAAGCTGCTGTACATCTTTAAGCAACCCTTCATGAGG
CCTGTGCAGACTACTCAAGAGGAGGACGGCTGTTCATGCCGGTTCCCAGAGGAGGAG
GAAGGCGGCTGCGAACTGCGCGTGAAATTCAGCCGCAGCGCAGATGCTCCAGCCTAC
AAGCAGGGGCAGAACCAGCTCTACAACGAACTCAATCTTGGTCGGAGAGAGGAGTAC
GACGTGCTGGACAAGCGGAGAGGACGGGACCCAGAAATGGGCGGGAAGCCGCGCAG
AAAGAATCCCCAAGAGGGCCTGTACAACGAGCTCCAAAAGGATAAGATGGCAGAAGC
CTATAGCGAGATTGGTATGAAAGGGGAACGCAGAAGAGGCAAAGGCCACGACGGACT
GTACCAGGGACTCAGCACCGCCACCAAGGACACCTATGACGCTCTTCACATGCAGGCC
CTGCCGCCTCGG [symbol] GGTTCCGGAGCCACGAACTTCTCTCTGTTAAAGCAAGCAG
GAGACGTGGAAGAAAACCCCGGTCCCATGAGAATCAGCAAGCCCCACCTGAGATCCA
TCAGCATCCAGTGCTACCTGTGCCTGCTGCTGAACAGCCACTTTCTGACAGAGGCCGG
CATCCACGTGTTCATCCTGGGCTGTTTTTCTGCCGGCCTGCCTAAGACCGAGGCCAACT
GGGTTAACGTGATCAGCGACCTGAAGAAGATCGAGGACCTGATCCAGAGCATGCACA
TCGACGCCACACTGTACACCGAGAGCGACGTGCACCCTAGCTGTAAAGTGACCGCCAT
GAAGTGCTTTCTGCTGGAACTGCAAGTGATCAGCCTGGAAAGCGGCGACGCCAGCATC
CACGACACCGTGGAAAACCTGATCATCCTGGCCAACAACAGCCTGAGCAGCAACGGC
AATGTGACCGAGTCCGGCTGCAAAGAGTGCGAGGAACTGGAAGAGAAGAATATCAAA
GAGTTCCTGCAGAGCTTCGTGCACATCGTGCAGATGTTCATCAACACCAGC [symbol] T
AACTCGAG,
(SEQ ID NO: 31)
ACTAGTGCCACCATGGCCAGACCTCATCCTTGGTGGCTGTGTGTGCTGGGCACACTCGT
TGGCCTGTCTGCTACACCAGCTCCTAAGAGCTGCCCCGAGAGACACTATTGGGCCCAG
GGCAAGCTGTGCTGCCAGATGTGTGAACCTGGCACCTTCCTGGTCAAGGACTGCGACC
AGCACAGAAAGGCCGCTCAGTGCGATCCTTGTATCCCCGGCGTGTCCTTCTCTCCCGAC
CACCACACAAGACCTCACTGCGAGAGCTGCAGACACTGCAATTCTGGACTGCTCGTGC
GGAACTGCACCATCACAGCCAATGCCGAGTGCGCCTGCAGAAATGGCTGGCAGTGCC
GGGACAAAGAATGTACCGAGTGCGACCCTCTGCCTAATCCTAGCCTGACCGCCAGAAG
CAGCCAGGCTTTGTCTCCTCATCCTCAGCCTACACATCTGCCCTACGTGTCCGAGATGC
TGGAAGCCAGAACAGCCGGCCATATGCAGACCCTGGCCGACTTTAGACAGCTGCCCGC
CAGAACACTGAGCACCCATTGGCCTCCACAGAGAAGCCTGTGCAGCAGCGACTTCATC
CGGATCCTCGTGATCTTCAGCGGCATGTTCCTGGTGTTCACACTGGCTGGCGCCCTGTT
TCTGCATAAGCGCGGTCGGAAGAAGCTGCTGTACATCTTTAAGCAACCCTTCATGAGG
CCTGTGCAGACTACTCAAGAGGAGGACGGCTGTTCATGCCGGTTCCCAGAGGAGGAG
GAAGGCGGCTGCGAACTGCGCGTGAAATTCAGCCGCAGCGCAGATGCTCCAGCCTAC
AAGCAGGGGCAGAACCAGCTCTACAACGAACTCAATCTTGGTCGGAGAGAGGAGTAC
GACGTGCTGGACAAGCGGAGAGGACGGGACCCAGAAATGGGCGGGAAGCCGCGCAG
AAAGAATCCCCAAGAGGGCCTGTACAACGAGCTCCAAAAGGATAAGATGGCAGAAGC
CTATAGCGAGATTGGTATGAAAGGGGAACGCAGAAGAGGCAAAGGCCACGACGGACT
GTACCAGGGACTCAGCACCGCCACCAAGGACACCTATGACGCTCTTCACATGCAGGCC
CTGCCGCCTCGG [symbol] GGATCTGGCGCCACCAATTTCAGCCTGCTGAAACAGGCTG
GCGACGTGGAAGAGAACCCTGGACCTATGAGAATCAGCAAGCCCCACCTGAGATCCA
TCAGCATCCAGTGCTACCTGTGCCTGCTGCTGAACAGCCACTTTCTGACAGAGGCCGG
CATCCACGTGTTCATCCTGGGCTGTTTTTCTGCCGGCCTGCCTAAGACCGAGGCCAACT
GGGTTAACGTGATCAGCGACCTGAAGAAGATCGAGGACCTGATCCAGAGCATGCACA
TCGACGCCACACTGTACACCGAGAGCGACGTGCACCCTAGCTGTAAAGTGACCGCCAT
GAAGTGCTTTCTGCTGGAACTGCAAGTGATCAGCCTGGAAAGCGGCGACGCCAGCATC
CACGACACCGTGGAAAACCTGATCATCCTGGCCAACAACAGCCTGAGCAGCAACGGC
AATGTGACCGAGTCCGGCTGCAAAGAGTGCGAGGAACTGGAAGAGAAGAATATCAAA
GAGTTCCTGCAGAGCTTCGTGCACATCGTGCAGATGTTCATCAACACCAGC [symbol] T
AACTCGAG,

optionally wherein any codon in SEQ ID NO: 22 or 31 may each independently be replaced by another codon encoding the same amino acid, preferably wherein the nucleic acid sequence is thereby codon optimised for expression in cells of a desired species, such as in human cells, according to codon optimisation principles known in the art, such as wherein the CAR-P2A-IL15 precursor is encoded by a nucleic acid comprising, consisting essentially of or consisting of a nucleic acid sequence at least 60%, at least 70%, at least 80%, at least 90%, or at least 95% identical to SEQ ID NO: 22 or 31.

Further, by means of an illustration, in certain particularly preferred embodiments, a CAR-P2A-IL21 precursor comprises, consists essentially of, or consists of the amino acid as set forth in SEQ ID NO: 32 below, with the signal sequence in bold font, the extracellular and intramembrane domains of CD27 in standard font, the 4-1BB intracellular co-stimulatory domain in italics, the CD3ζ intracellular activation domain underlined, the GSG-P2A self-cleaving peptide in bold italics, and the IL-21 bold underlined:

(SEQ ID NO: 32)
MARPHPWWLCVLGTLVGLSATPAPKSCPERHYWAQGKLCCQMCEPGTFLVKDC
DQHRKAAQCDPCIPGVSFSPDHHTRPHCESCRHCNSGLLVRNCTITANAECAC
RNGWQCRDKECTECDPLPNPSLTARSSQALSPHPQPTHLPYVSEMLEARTAGH
MQTLADFRQLPARTLSTHWPPQRSLCSSDFIRILVIFSGMFLVFTLAGALFLH
KRGRKKLLYIFKQPFMRPVQTTQEEDGCSCRFPEEEEGGCELRVKFSRSADAP
AYKQGQNQLYNELNLGRREEYDVLDKRRGRDPEMGGKPRRKNPQEGLYNELQK
DKMAEAYSEIGMKGERRRGKGHDGLYQGLSTATKDTYDALHMQALPPREF [symbol] [symbol]
MRSSPGNMERIVICLMVIFLGTLVHKSSSQGQDR
HMIRMRQLIDIVDQLKNYVNDLVPEFLPAPEDVETNCEWSAFSCFQKAQLKSA
NTGNNERIINVSIKKLKRKPPSTNAGRRQKHRLTCPSCDSYEKKPPKEFLERF
KSLLQKMIHQHLSSRTHGSEDSEF.

In certain embodiments the CAR-P2A-IL21 precursor comprises, consists essentially of or consists of an amino acid sequence at least 80% identical, preferably at least 85% identical, more preferably at least 90% identical, even more preferably at least 95% identical, such as particularly preferably at least 96%, at least 97%, at least 98% or at least 99% or 100% identical to SEQ ID NO: 32.

In certain embodiments, the CAR-P2A-IL21 precursor form as taught herein, such as in particular the one set forth in SEQ ID NO: 32, may be encoded by a nucleic acid comprising, consisting essentially of or consisting of the nucleic acid sequence set forth in SEQ ID NO: 33, with the ATG start and TAA stop codons of the CAR-P2A-IL21 coding sequence in bold, an SpeI restriction site underlined, GCCACC Kozak sequence in italics, and an XhoI restriction site double underlined, and the EcoRI restriction site flanking the IL-21 cassette in bold italics (evidently, other restriction sites may be included, or none, depending on experimental convenience):

(SEQ ID NO: 33)
ACTAGTGCCACCATGGCCAGACCTCATCCTTGGTGGCTGTGTGTGCTGGGCACACTCGT
TGGCCTGTCTGCTACACCAGCTCCTAAGAGCTGCCCCGAGAGACACTATTGGGCCCAG
GGCAAGCTGTGCTGCCAGATGTGTGAACCTGGCACCTTCCTGGTCAAGGACTGCGACC
AGCACAGAAAGGCCGCTCAGTGCGATCCTTGTATCCCCGGCGTGTCCTTCTCTCCCGAC
CACCACACAAGACCTCACTGCGAGAGCTGCAGACACTGCAATTCTGGACTGCTCGTGC
GGAACTGCACCATCACAGCCAATGCCGAGTGCGCCTGCAGAAATGGCTGGCAGTGCC
GGGACAAAGAATGTACCGAGTGCGACCCTCTGCCTAATCCTAGCCTGACCGCCAGAAG
CAGCCAGGCTTTGTCTCCTCATCCTCAGCCTACACATCTGCCCTACGTGTCCGAGATGC
TGGAAGCCAGAACAGCCGGCCATATGCAGACCCTGGCCGACTTTAGACAGCTGCCCGC
CAGAACACTGAGCACCCATTGGCCTCCACAGAGAAGCCTGTGCAGCAGCGACTTCATC
CGGATCCTCGTGATCTTCAGCGGCATGTTCCTGGTGTTCACACTGGCTGGCGCCCTGTT
TCTGCATAAGCGCGGTCGGAAGAAGCTGCTGTACATCTTTAAGCAACCCTTCATGAGG
CCTGTGCAGACTACTCAAGAGGAGGACGGCTGTTCATGCCGGTTCCCAGAGGAGGAG
GAAGGCGGCTGCGAACTGCGCGTGAAATTCAGCCGCAGCGCAGATGCTCCAGCCTAC
AAGCAGGGGCAGAACCAGCTCTACAACGAACTCAATCTTGGTCGGAGAGAGGAGTAC
GACGTGCTGGACAAGCGGAGAGGACGGGACCCAGAAATGGGCGGGAAGCCGCGCAG
AAAGAATCCCCAAGAGGGCCTGTACAACGAGCTCCAAAAGGATAAGATGGCAGAAGC
CTATAGCGAGATTGGTATGAAAGGGGAACGCAGAAGAGGCAAAGGCCACGACGGACT
GTACCAGGGACTCAGCACCGCCACCAAGGACACCTATGACGCTCTTCACATGCAGGCC
CTGCCGCCTCGG [symbol] GGATCTGGCGCCACCAATTTCAGCCTGCTGAAACAGGCTG
GCGACGTGGAAGAGAACCCTGGACCTATGAGATCCAGTCCTGGCAACATGGAGAGGA
TTGTCATCTGTCTGATGGTCATCTTCTTGGGGACACTGGTCCACAAATCAAGCTCCCAA
GGTCAAGATCGCCACATGATTAGAATGCGTCAACTTATAGATATTGTTGATCAGCTGA
AAAATTATGTGAATGACTTGGTCCCTGAATTTCTGCCAGCTCCAGAAGATGTAGAGAC
AAACTGTGAGTGGTCAGCTTTTTCCTGCTTTCAGAAGGCCCAACTAAAGTCAGCAAAT
ACAGGAAACAATGAAAGGATAATCAATGTATCAATTAAAAAGCTGAAGAGGAAACCA
CCTTCCACAAATGCAGGGAGAAGACAGAAACACAGACTAACATGCCCTTCATGTGATT
CTTATGAGAAAAAACCACCCAAAGAATTCCTAGAAAGATTCAAATCACTTCTCCAAAA
GATGATTCATCAGCATCTGTCCTCTAGAACACACGGAAGTGAAGATTCC [symbol] TAA
CTCGAG,

optionally wherein any codon in SEQ ID NO: 33 may each independently be replaced by another codon encoding the same amino acid, preferably wherein the nucleic acid sequence is thereby codon optimised for expression in cells of a desired species, such as in human cells, according to codon optimisation principles known in the art, such as wherein the CAR-P2A-IL21 precursor is encoded by a nucleic acid comprising, consisting essentially of or consisting of a nucleic acid sequence at least 60%, at least 70%, at least 80%, at least 90%, or at least 95% identical to SEQ ID NO: 33.

Further, by means of an illustration, in certain particularly preferred embodiments, a CAR-P2A-IL12 precursor comprises, consists essentially of, or consists of the amino acid as set forth in SEQ ID NO: 34 below, with the signal sequence in bold font, the extracellular and intramembrane domains of CD27 in standard font, the 4-1BB intracellular co-stimulatory domain in italics, the CD3ζ intracellular activation domain underlined, the GSG-P2A self-cleaving peptide in bold italics, and the IL-12A and 12B (in that order, and separated by a GSG-P2A peptide) bold underlined:

(SEQ ID NO: 34)
MARPHPWWLCVLGTLVGLSATPAPKSCPERHYWAQGKLCCQMCEPGTFLVKDCDQHR
KAAQCDPCIPGVSFSPDHHTRPHCESCRHCNSGLLVRNCTITANAECACRNGWQCRDKEC
TECDPLPNPSLTARSSQALSPHPQPTHLPYVSEMLEARTAGHMQTLADFRQLPARTLSTHW
PPQRSLCSSDFIRILVIFSGMFLVFTLAGALFLHKRGRKKLLYIFKQPFMRPVQTTQEEDGCSC
RFPEEEEGGCELRVKFSRSADAPAYKQGQNQLYNELNLGRREEYDVLDKRRGRDPEMGG
KPRRKNPQEGLYNELQKDKMAEAYSEIGMKGERRRGKGHDGLYQGLSTATKDTYDALH
MQALPPREF [symbol] [symbol] MCPARSLLLVATLVLLDHLSLARNLPVA
TPDPGMFPCLHHSQNLLRAVSNMLQKARQTLEFYPCTSEEIDHEDITKDKTSTVEACL
PLELTKNESCLNSRETSFITNGSCLASRKTSFMMALCLSSIYEDLKMYQVEFKTMNAK
LLMDPKRQIFLDQNMLAVIDELMQALNFNSETVPQKSSLEEPDFYKTKIKLCILLHAF
RIRAVTIDRVMSYLNAS [symbol] [symbol] MCHQQLVISWFSLVFLASPL
VAIWELKKDVYVVELDWYPDAPGEMVVLTCDTPEEDGITWTLDQSSEVLGSGKTLTI
QVKEFGDAGQYTCHKGGEVLSHSLLLLHKKEDGIWSTDILKDQKEPKNKTFLRCEA
KNYSGRFTCWWLTTISTDLTFSVKSSRGSSDPQGVTCGAATLSAERVRGDNKEYEYS
VECQEDSACPAAEESLPIEVMVDAVHKLKYENYTSSFFIRDIIKPDPPKNLQLKPLKNS
RQVEVSWEYPDTWSTPHSYFSLTFCVQVQGKSKREKKDRVFTDKTSATVICRKNASIS
VRAQDRYYSSSWSEWASVPCSEF.

In certain embodiments the CAR-P2A-IL12 precursor comprises, consists essentially of or consists of an amino acid sequence at least 80% identical, preferably at least 85% identical, more preferably at least 90% identical, even more preferably at least 95% identical, such as particularly preferably at least 96%, at least 97%, at least 98% or at least 99% or 100% identical to SEQ ID NO: 34.

In certain embodiments, the CAR-P2A-IL12 precursor form as taught herein, such as in particular the one set forth in SEQ ID NO: 34, may be encoded by a nucleic acid comprising, consisting essentially of or consisting of the nucleic acid sequence set forth in SEQ ID NO: 35, with the ATG start and TAA stop codons of the CAR-P2A-IL12A-P2A-IL12B coding sequence in bold, an SpeI restriction site underlined, GCCACC Kozak sequence in italics, and an XhoI restriction site double underlined, and the EcoRI restriction site flanking the IL-12 cassette in bold italics (evidently, other restriction sites may be included, or none, depending on experimental convenience):

(SEQ ID NO: 35)
ACTAGTGCCACCATGGCCAGACCTCATCCTTGGTGGCTGTGTGTGCTGGGCACACTCGT
TGGCCTGTCTGCTACACCAGCTCCTAAGAGCTGCCCCGAGAGACACTATTGGGCCCAG
GGCAAGCTGTGCTGCCAGATGTGTGAACCTGGCACCTTCCTGGTCAAGGACTGCGACC
AGCACAGAAAGGCCGCTCAGTGCGATCCTTGTATCCCCGGCGTGTCCTTCTCTCCCGAC
CACCACACAAGACCTCACTGCGAGAGCTGCAGACACTGCAATTCTGGACTGCTCGTGC
GGAACTGCACCATCACAGCCAATGCCGAGTGCGCCTGCAGAAATGGCTGGCAGTGCC
GGGACAAAGAATGTACCGAGTGCGACCCTCTGCCTAATCCTAGCCTGACCGCCAGAAG
CAGCCAGGCTTTGTCTCCTCATCCTCAGCCTACACATCTGCCCTACGTGTCCGAGATGC
TGGAAGCCAGAACAGCCGGCCATATGCAGACCCTGGCCGACTTTAGACAGCTGCCCGC
CAGAACACTGAGCACCCATTGGCCTCCACAGAGAAGCCTGTGCAGCAGCGACTTCATC
CGGATCCTCGTGATCTTCAGCGGCATGTTCCTGGTGTTCACACTGGCTGGCGCCCTGTT
TCTGCATAAGCGCGGTCGGAAGAAGCTGCTGTACATCTTTAAGCAACCCTTCATGAGG
CCTGTGCAGACTACTCAAGAGGAGGACGGCTGTTCATGCCGGTTCCCAGAGGAGGAG
GAAGGCGGCTGCGAACTGCGCGTGAAATTCAGCCGCAGCGCAGATGCTCCAGCCTAC
AAGCAGGGGCAGAACCAGCTCTACAACGAACTCAATCTTGGTCGGAGAGAGGAGTAC
GACGTGCTGGACAAGCGGAGAGGACGGGACCCAGAAATGGGCGGGAAGCCGCGCAG
AAAGAATCCCCAAGAGGGCCTGTACAACGAGCTCCAAAAGGATAAGATGGCAGAAGC
CTATAGCGAGATTGGTATGAAAGGGGAACGCAGAAGAGGCAAAGGCCACGACGGACT
GTACCAGGGACTCAGCACCGCCACCAAGGACACCTATGACGCTCTTCACATGCAGGCC
CTGCCGCCTCGG [symbol] GGATCTGGCGCCACCAATTTCAGCCTGCTGAAACAGGCTG
GCGACGTGGAAGAGAACCCTGGACCTATGTGTCCAGCGCGCAGCCTCCTCCTTGTGGC
TACCCTGGTCCTCCTGGACCACCTCAGTTTGGCCAGAAACCTCCCCGTGGCCACTCCAG
ACCCAGGAATGTTCCCATGCCTTCACCACTCCCAAAACCTGCTGAGGGCCGTCAGCAA
CATGCTCCAGAAGGCCAGACAAACTCTAGAATTTTACCCTTGCACTTCTGAAGAGATT
GATCATGAAGATATCACAAAAGATAAAACCAGCACAGTGGAGGCCTGTTTACCATTGG
AATTAACCAAGAATGAGAGTTGCCTAAATTCCAGAGAGACCTCTTTCATAACTAATGG
GAGTTGCCTGGCCTCCAGAAAGACCTCTTTTATGATGGCCCTGTGCCTTAGTAGTATTT
ATGAAGACTTGAAGATGTACCAGGTGGAGTTCAAGACCATGAATGCAAAGCTTCTGAT
GGATCCTAAGAGGCAGATCTTTCTAGATCAAAACATGCTGGCAGTTATTGATGAGCTG
ATGCAGGCCCTGAATTTCAACAGTGAGACTGTGCCACAAAAATCCTCCCTTGAAGAAC
CGGATTTTTATAAAACTAAAATCAAGCTCTGCATACTTCTTCATGCTTTCAGAATTCGG
GCAGTGACTATTGATAGAGTGATGAGCTATCTGAATGCTTCCGGATCTGGCGCCACCA
ATTTCAGCCTGCTGAAACAGGCTGGCGACGTGGAAGAGAACCCTGGACCTATGTGTCA
CCAGCAGTTGGTCATCTCTTGGTTTTCCCTGGTTTTTCTGGCATCTCCCCTCGTGGCCAT
ATGGGAACTGAAGAAAGATGTTTATGTCGTAGAATTGGATTGGTATCCGGATGCCCCT
GGAGAAATGGTGGTCCTCACCTGTGACACCCCTGAAGAAGATGGTATCACCTGGACCT
TGGACCAGAGCAGTGAGGTCTTAGGCTCTGGCAAAACCCTGACCATCCAAGTCAAAGA
GTTTGGAGATGCTGGCCAGTACACCTGTCACAAAGGAGGCGAGGTTCTAAGCCATTCG
CTCCTGCTGCTTCACAAAAAGGAAGATGGAATTTGGTCCACTGATATTTTAAAGGACC
AGAAAGAACCCAAAAATAAGACCTTTCTAAGATGCGAGGCCAAGAATTATTCTGGAC
GTTTCACCTGCTGGTGGCTGACGACAATCAGTACTGATTTGACATTCAGTGTCAAAAG
CAGCAGAGGCTCTTCTGACCCCCAAGGGGTGACGTGCGGAGCTGCTACACTCTCTGCA
GAGAGAGTCAGAGGGGACAACAAGGAGTATGAGTACTCAGTGGAGTGCCAGGAGGAC
AGTGCCTGCCCAGCTGCTGAGGAGAGTCTGCCCATTGAGGTCATGGTGGATGCCGTTC
ACAAGCTCAAGTATGAAAACTACACCAGCAGCTTCTTCATCAGGGACATCATCAAACC
TGACCCACCCAAGAACTTGCAGCTGAAGCCATTAAAGAATTCTCGGCAGGTGGAGGTC
AGCTGGGAGTACCCTGACACCTGGAGTACTCCACATTCCTACTTCTCCCTGACATTCTG
CGTTCAGGTCCAGGGCAAGAGCAAGAGAGAAAAGAAAGATAGAGTCTTCACGGACAA
GACCTCAGCCACGGTCATCTGCCGCAAAAATGCCAGCATTAGCGTGCGGGCCCAGGAC
CGCTACTATAGCTCATCTTGGAGCGAATGGGCATCTGTGCCCTGCAGT [symbol] TAACT
CGAG,

optionally wherein any codon in SEQ ID NO: 35 may each independently be replaced by another codon encoding the same amino acid, preferably wherein the nucleic acid sequence is thereby codon optimised for expression in cells of a desired species, such as in human cells, according to codon optimisation principles known in the art, such as wherein the CAR-P2A-IL21 precursor is encoded by a nucleic acid comprising, consisting essentially of or consisting of a nucleic acid sequence at least 60%, at least 70%, at least 80%, at least 90%, or at least 95% identical to SEQ ID NO: 35.

A further aspect of the invention provides a method for producing the NK cell as taught herein, comprising introducing a nucleic acid encoding the CAR as defined herein in an expressible format, and optionally a nucleic acid encoding one or more immunostimulatory cytokines in an expressible format, into a starting population of NK cells.

In certain embodiments, the nucleic acid(s) introduced into the cells may be DNA expression cassette(s) or vector(s), from which mRNA(s) encoding the CAR and optionally the cytokine, optionally within a single ORF as explained above, can be transcribed using the cell's transcription machinery. In other embodiments, the nucleic acid(s) introduced into the cells may be mRNA(s) encoding the CAR and optionally the cytokine, optionally within a single ORF as explained above, which can be translated by the cell's protein translation machinery. In certain preferred embodiments, the nucleic acid(s) such as mRNA(s) may be introduced into the starting population of NK cells by electroporation.

Optionally but not necessarily the method may further comprise selecting and/or expanding NK cells which comprise said nucleic acid or nucleic acids and which are capable of expressing the CAR and optionally the one or more immunostimulatory cytokine. Methods of selecting transfected or transduced cells are routine in the art and may use established positive selection markers, such as genes conferring resistance to blasticidin, geneticin, hygromycin B, puromycin or zeocin.

In certain particularly preferred embodiments, the method may involve transient transfection of the NK cells, such as with a DNA construct or an mRNA molecule, preferably an mRNA molecule. In certain embodiments, the transient transfection may not require any selection of the transfected cells. In certain embodiments, the method may involve stable transfection of the NK cells, in which selection of the engineered cells may be desired.

A further aspect provides a pharmaceutical composition comprising the engineered NK cell as taught herein and a pharmaceutically acceptable carrier.

The term “pharmaceutically acceptable” as used herein is consistent with the art and means compatible with the other ingredients of a pharmaceutical composition and not deleterious to the recipient thereof.

As used herein, “carrier” or “excipient” includes any and all solvents, diluents, buffers (e.g., neutral buffered saline or phosphate buffered saline), solubilizers, colloids, dispersion media, vehicles, fillers, chelating agents (e.g., EDTA or glutathione), amino acids (e.g., glycine), proteins, disintegrants, binders, lubricants, wetting agents, emulsifiers, sweeteners, colorants, flavourings, aromatisers, thickeners, agents for achieving a depot effect, coatings, antifungal agents, preservatives, stabilisers, antioxidants, tonicity controlling agents, absorption delaying agents, and the like. The use of such media and agents for pharmaceutical active substances is well known in the art. Such materials should be non-toxic and should not interfere with the activity of the cells.

The precise nature of the carrier or excipient or other material will depend on the route of administration. For example, the composition may be in the form of a parenterally acceptable aqueous solution, which is pyrogen-free and has suitable pH, isotonicity and stability. For general principles in medicinal formulation, the reader is referred to Cell Therapy: Stem Cell Transplantation, Gene Therapy, and Cellular Immunotherapy, by G. Morstyn & W. Sheridan eds., Cambridge University Press, 1996; and Hematopoietic Stem Cell Therapy, E. D. Ball, J. Lister & P. Law, Churchill Livingstone, 2000.

Liquid pharmaceutical compositions may generally include a liquid carrier such as water or a pharmaceutically acceptable aqueous solution. For example, physiological saline solution, tissue or cell culture media, dextrose or other saccharide solution or glycols such as ethylene glycol, propylene glycol or polyethylene glycol may be included.

The composition may include one or more cell protective molecules, cell regenerative molecules, growth factors, anti-apoptotic factors or factors that regulate gene expression in the cells. Such substances may render the cells independent of its environment.

Such pharmaceutical compositions may contain further components ensuring the viability of the cells therein. For example, the compositions may comprise a suitable buffer system (e.g., phosphate or carbonate buffer system) to achieve desirable pH, more usually near neutral pH, and may comprise sufficient salt to ensure isosmotic conditions for the cells to prevent osmotic stress. For example, suitable solution for these purposes may be phosphate-buffered saline (PBS), sodium chloride solution, Ringer's Injection or Lactated Ringer's Injection, as known in the art. Further, the composition may comprise a carrier protein, e.g., albumin (e.g., bovine or human albumin), which may increase the viability of the cells.

Further suitably pharmaceutically acceptable carriers or additives are well known to those skilled in the art and for instance may be selected from proteins such as collagen or gelatine, carbohydrates such as starch, polysaccharides, sugars (dextrose, glucose and sucrose), cellulose derivatives like sodium or calcium carboxymethylcellulose, hydroxypropyl cellulose or hydroxypropylmethyl cellulose, pregeletanized starches, pectin agar, carrageenan, clays, hydrophilic gums (acacia gum, guar gum, arabic gum and xanthan gum), alginic acid, alginates, hyaluronic acid, polyglycolic and polylactic acid, dextran, pectins, synthetic polymers such as water-soluble acrylic polymer or polyvinylpyrrolidone, proteoglycans, calcium phosphate and the like.

In an embodiment, the pharmaceutical cell preparation as defined above may be administered in a form of liquid composition. In embodiments, the cells or pharmaceutical composition comprising such can be administered systemically, topically, within an organ, at a site of organ dysfunction or lesion or at a site of tissue lesion.

Preferably, the pharmaceutical compositions may comprise a therapeutically effective amount of the desired cells. The term “therapeutically effective amount” refers to an amount which can elicit a biological or medicinal response in a tissue, system, animal or human that is being sought by a researcher, veterinarian, medical doctor or other clinician, and in particular can prevent or alleviate one or more of the local or systemic symptoms or features of a disease or condition being treated. Appropriate therapeutically effective amounts may be determined by a qualified physician with due regard to the nature of the desired cells, the disease condition and severity, and the age, size and condition of the subject.

Also provided are methods of producing said pharmaceutical compositions by admixing the cells of the invention with one or more additional components as described above as well as with one or more pharmaceutical excipients as described above.

Also disclosed is an arrangement or kit of parts comprising a surgical instrument or device for administration of the cells as taught herein or the pharmaceutical compositions as defined herein to a subject, such as for example systemically, for example, by injection, and further comprising the cells as taught herein or the pharmaceutical compositions as defined herein.

In an embodiment, the pharmaceutical composition as define above may be administered in a form of a liquid composition.

The quantity of cells to be administered will vary for the subject being treated. In certain embodiments, the quantity of cells to be administered is between 10² to 10¹⁰ or between 10² to 10⁹, or between 10³ to 10¹⁰ or between 10³ to 10⁹, or between 10⁴ to 10¹⁰ or between 10⁴ to 10⁹, such as between 10⁴ and 10⁸, or between 10⁵ and 10⁷, e.g., about 1×10⁵, about 5×10⁵, about 1×10⁶, about 5×10⁶, about 1×10⁷, about 5×10⁷, about 1×10⁸, about 5×10⁸, about 1×10⁹, about 5×10⁹, or about 1×10¹ cells can be administered to a human subject. For example, such administration may be suitably distributed over one or more doses (e.g., distributed over 2, 3, 4, 5, 6, 7, 8 9 or 10 or more doses) administered over one or more days (e.g., over 1, 2, 3, 4 or 5 or more days). However, the precise determination of a therapeutically effective dose may be based on factors individual to each patient, including their size, age, tissue damage, and can be readily ascertained by those skilled in the art from this disclosure and the knowledge in the art. Suitably but without limitation, in a composition to be administered, cells may be present at a concentration between about 10⁴/ml to about 10⁹/ml, preferably between about 10⁵/ml and about 10⁸/ml, yet more preferably between about 1×10⁶/ml and about 1×10⁸/ml.

A further aspect provides the engineered NK cells or the pharmaceutical composition as taught herein for use in therapy. Particularly provided are the engineered NK cells or the pharmaceutical composition as taught herein for use in a method of treating a neoplastic disease. Particularly provided are the engineered NK cells or the pharmaceutical composition as taught herein for use in a method of treating cancer.

Reference to “therapy” or “treatment” broadly encompasses both curative and preventative treatments, and the terms may particularly refer to the alleviation or measurable lessening of one or more symptoms or measurable markers of a pathological condition such as a disease or disorder. The terms encompass primary treatments as well as neo-adjuvant treatments, adjuvant treatments and adjunctive therapies. Measurable lessening includes any statistically significant decline in a measurable marker or symptom. Generally, the terms encompass both curative treatments and treatments directed to reduce symptoms and/or slow progression of the disease. The terms encompass both the therapeutic treatment of an already developed pathological condition, as well as prophylactic or preventative measures, wherein the aim is to prevent or lessen the chances of incidence of a pathological condition. In certain embodiments, the terms may relate to therapeutic treatments. In certain other embodiments, the terms may relate to preventative treatments. Treatment of a chronic pathological condition during the period of remission may also be deemed to constitute a therapeutic treatment. The term may encompass ex vivo or in vivo treatments as appropriate in the context of the present invention.

The terms “subject”, “individual” or “patient” are used interchangeably throughout this specification, and typically and preferably denote humans, but may also encompass reference to non-human animals, preferably warm-blooded animals, even more preferably non-human mammals. Particularly preferred are human subjects including both genders and all age categories thereof. In other embodiments, the subject is an experimental animal or animal substitute as a disease model. The term does not denote a particular age or sex. Thus, adult and newborn subjects, as well as fetuses, whether male or female, are intended to be covered. The term subject is further intended to include transgenic non-human species.

The term “subject in need of treatment” or similar as used herein refers to subjects diagnosed with or having a disease as recited herein and/or those in whom said disease is to be prevented.

The term “neoplastic disease” generally refers to any disease or disorder characterised by neoplastic cell growth and proliferation, whether benign (not invading surrounding normal tissues, not forming metastases), pre-malignant (pre-cancerous), or malignant (invading adjacent tissues and capable of producing metastases). The term neoplastic disease generally includes all transformed cells and tissues and all cancerous cells and tissues. Neoplastic diseases or disorders include, but are not limited to abnormal cell growth, benign tumors, premalignant or precancerous lesions, malignant tumors, and cancer. Examples of neoplastic diseases or disorders are benign, pre-malignant, or malignant neoplasms located in any tissue or organ, such as in the prostate, colon, abdomen, bone, breast, digestive system, liver, pancreas, peritoneum, endocrine glands (adrenal, parathyroid, pituitary, testicles, ovary, thymus, thyroid), eye, head and neck, nervous (central and peripheral), lymphatic system, pelvic, skin, soft tissue, spleen, thoracic, or urogenital tract.

As used herein, the terms “tumor” or “tumor tissue” refer to an abnormal mass of tissue that results from excessive cell division. A tumor or tumor tissue comprises tumor cells which are neoplastic cells with abnormal growth properties and no useful bodily function. Tumors, tumor tissue and tumor cells may be benign, pre-malignant or malignant, or may represent a lesion without any cancerous potential. A tumor or tumor tissue may also comprise tumor-associated non-tumor cells, e.g., vascular cells which form blood vessels to supply the tumor or tumor tissue. Non-tumor cells may be induced to replicate and develop by tumor cells, for example, the induction of angiogenesis in a tumor or tumor tissue.

As used herein, the term “cancer” refers to a malignant neoplasm characterised by deregulated or unregulated cell growth. The term “cancer” includes primary malignant cells or tumors (e.g., those whose cells have not migrated to sites in the subject's body other than the site of the original malignancy or tumor) and secondary malignant cells or tumors (e.g., those arising from metastasis, the migration of malignant cells or tumor cells to secondary sites that are different from the site of the original tumor). The term “metastatic” or “metastasis” generally refers to the spread of a cancer from one organ or tissue to another non-adjacent organ or tissue. The occurrence of the neoplastic disease in the other non-adjacent organ or tissue is referred to as metastasis.

Examples of cancer include but are not limited to carcinoma, lymphoma, blastoma, sarcoma, and leukemia or lymphoid malignancies. More particular examples of such cancers include without limitation: squamous cell cancer (e.g., epithelial squamous cell cancer), lung cancer including small-cell lung cancer, non-small cell lung cancer, adenocarcinoma of the lung, squamous carcinoma of the lung and large cell carcinoma of the lung, cancer of the peritoneum, hepatocellular cancer, gastric or stomach cancer including gastrointestinal cancer, pancreatic cancer, glioma, glioblastoma, cervical cancer, ovarian cancer, liver cancer, bladder cancer, hepatoma, breast cancer, colon cancer, rectal cancer, colorectal cancer, endometrial cancer or uterine carcinoma, salivary gland carcinoma, kidney or renal cancer, prostate cancer, vulvar cancer, thyroid cancer, hepatic carcinoma, anal carcinoma, penile carcinoma, as well as CNS cancer, melanoma, head and neck cancer, bone cancer, bone marrow cancer, duodenum cancer, esophageal cancer, thyroid cancer, or hematological cancer.

Other non-limiting examples of cancers or malignancies include, but are not limited to: Acute Childhood Lymphoblastic Leukemia, Acute Lymphoblastic Leukemia, Acute Lymphocytic Leukemia, Acute Myeloid Leukemia, Adrenocortical Carcinoma, Adult (Primary) Hepatocellular Cancer, Adult (Primary) Liver Cancer, Adult Acute Lymphocytic Leukemia, Adult Acute Myeloid Leukemia, Adult Hodgkin's Disease, Adult Hodgkin's Lymphoma, Adult Lymphocytic Leukemia, Adult Non-Hodgkin's Lymphoma, Adult Primary Liver Cancer, Adult Soft Tissue Sarcoma, AIDS-Related Lymphoma, AIDS-Related Malignancies, Anal Cancer, Astrocytoma, Bile Duct Cancer, Bladder Cancer, Bone Cancer, Brain Stem Glioma, Brain Tumors, Breast Cancer, Cancer of the Renal Pelvis and Urethra, Central Nervous System (Primary) Lymphoma, Central Nervous System Lymphoma, Cerebellar Astrocytoma, Cerebral Astrocytoma, Cervical Cancer, Childhood (Primary) Hepatocellular Cancer, Childhood (Primary) Liver Cancer, Childhood Acute Lymphoblastic Leukemia, Childhood Acute Myeloid Leukemia, Childhood Brain Stem Glioma, Glioblastoma, Childhood Cerebellar Astrocytoma, Childhood Cerebral Astrocytoma, Childhood Extracranial Germ Cell Tumors, Childhood Hodgkin's Disease, Childhood Hodgkin's Lymphoma, Childhood Hypothalamic and Visual Pathway Glioma, Childhood Lymphoblastic Leukemia, Childhood Medulloblastoma, Childhood Non-Hodgkin's Lymphoma, Childhood Pineal and Supratentorial Primitive Neuroectodermal Tumors, Childhood Primary Liver Cancer, Childhood Rhabdomyosarcoma, Childhood Soft Tissue Sarcoma, Childhood Visual Pathway and Hypothalamic Glioma, Chronic Lymphocytic Leukemia, Chronic Myelogenous Leukemia, Colon Cancer, Cutaneous T-Cell Lymphoma, Endocrine Pancreas Islet Cell Carcinoma, Endometrial Cancer, Ependymoma, Epithelial Cancer, Esophageal Cancer, Ewing's Sarcoma and Related Tumors, Exocrine Pancreatic Cancer, Extracranial Germ Cell Tumor, Extragonadal Germ Cell Tumor, Extrahepatic Bile Duct Cancer, Eye Cancer, Female Breast Cancer, Gallbladder Cancer, Gastric Cancer, Gastrointestinal Carcinoid Tumor, Gastrointestinal Tumors, Germ Cell Tumors, Gestational Trophoblastic Tumor, Hairy Cell Leukemia, Head and Neck Cancer, Hepatocellular Cancer, Hodgkin's Disease, Hodgkin's Lymphoma, Hypergammaglobulinemia, Hypopharyngeal Cancer, Intestinal Cancers, Intraocular Melanoma, Islet Cell Carcinoma, Islet Cell Pancreatic Cancer, Kaposi's Sarcoma, Kidney Cancer, Laryngeal Cancer, Lip and Oral Cavity Cancer, Liver Cancer, Lung Cancer, Lymphoproliferative Disorders, Macroglobulinemia, Male Breast Cancer, Malignant Mesothelioma, Malignant Thymoma, Medulloblastoma, Melanoma, Mesothelioma, Metastatic Occult Primary Squamous Neck Cancer, Metastatic Primary Squamous Neck Cancer, Metastatic Squamous Neck Cancer, Multiple Myeloma, Multiple Myeloma/Plasma Cell Neoplasm, Myelodysplastic Syndrome, Myelogenous Leukemia, Myeloid Leukemia, Myeloproliferative Disorders, Nasal Cavity and Paranasal Sinus Cancer, Nasopharyngeal Cancer, Neuroblastoma, Non-Hodgkin's Lymphoma During Pregnancy, Non-melanoma Skin Cancer, Non-Small Cell Lung Cancer, Occult Primary Metastatic Squamous Neck Cancer, Oropharyngeal Cancer, Osteo-/Malignant Fibrous Sarcoma, Osteosarcoma/Malignant Fibrous Histiocytoma, Osteosarcoma/Malignant Fibrous Histiocytoma of Bone, Ovarian Epithelial Cancer, Ovarian Germ Cell Tumour, Ovarian Low Malignant Potential Tumor, Pancreatic Cancer, Paraproteinemias, Purpura, Parathyroid Cancer, Penile Cancer, Pheochromocytoma, Pituitary Tumor, Plasma Cell Neoplasm/Multiple Myeloma, Primary Central Nervous System Lymphoma, Primary Liver Cancer, Prostate Cancer, Rectal Cancer, Renal Cell Cancer, Renal Pelvis and Urethra Cancer, Retinoblastoma, Rhabdomyosarcoma, Salivary Gland Cancer, Sarcoidosis Sarcomas, Sezary Syndrome, Skin Cancer, Small Cell Lung Cancer, Small Intestine Cancer, Soft Tissue Sarcoma, Squamous Neck Cancer, Stomach Cancer, Supratentorial Primitive Neuroectodermal and Pineal Tumors, T-Cell Lymphoma, Testicular Cancer, Thymoma, Thyroid Cancer, Transitional Cell Cancer of the Renal Pelvis and Urethra, Transitional Renal Pelvis and Urethra Cancer, Trophoblastic Tumours, Urethra and Renal Pelvis Cell Cancer, Urethral Cancer, Uterine Cancer, Uterine Sarcoma, Vaginal Cancer, Visual Pathway and Hypothalamic Glioma, Vulvar Cancer, Waldenstrom's Macroglobulinemia, or Wilms' Tumour.

In certain embodiments, the cancer is a haematological malignancy. In certain embodiments, the cancer is Burkitt lymphoma. In certain embodiments, the cancer is a solid malignancy (solid tumor). In certain embodiments, the cancer is colorectal cancer or pancreatic cancer. In certain embodiments, where the cancer is haematological malignancy, the NK cell need not be, but may be, engineered to further express the one or more immunostimulatory cytokine. In certain embodiments, where the cancer is solid tumor, the NK cell need not be, but preferably is, engineered to further express the one or more immunostimulatory cytokine, such as one or more immunostimulatory interleukin, such as IL-15 and/or IL-21, such as particularly preferably at least IL-15 or only IL-15. The inventors have demonstrated that co-expression of the immunostimulatory interleukin(s) with the CAR in the NK cell greatly improves the ability of the NK cell to target cells of solid tumors, including cancer cells and cells of the tumor environment, such as CAFs.

In certain embodiments, the neoplastic disease such as cancer comprises CD70-positive cancerous cells. Cells such as tumor cells or cells of the tumor microenvironment as disclosed herein may in the context of the present specification be said to “comprise the expression” or conversely to “not express” one or more markers, such as one or more genes, polypeptides or proteins, such as CD70, or be described as “positive” (+) or conversely as “negative” (−) for one or more markers, such as one or more genes, polypeptides or proteins, such as CD70.

Such terms are commonplace and well-understood by the skilled person when characterising cell phenotypes. By means of additional guidance, when a cell is said to be positive for or to express or comprise expression of a given marker, such as a given gene, polypeptide or protein, such as CD70, a skilled person would conclude the presence or evidence of a distinct signal for the marker when carrying out a measurement capable of detecting or quantifying the marker in or on the cell. Suitably, the presence or evidence of the distinct signal for the marker would be concluded based on a comparison of the measurement result obtained for the cell to a result of the same measurement carried out for a negative control (for example, a cell known to not express the marker) and/or a positive control (for example, a cell known to express the marker). Where the measurement method allows for a quantitative assessment of the marker, a positive cell may generate a signal for the marker that is at least 1.5-fold higher than a signal generated for the marker by a negative control cell or than an average signal generated for the marker by a population of negative control cells, e.g., at least 2-fold, at least 4-fold, at least 10-fold, at least 20-fold, at least 30-fold, at least 40-fold, at least 50-fold higher or even higher. Further, a positive cell may generate a signal for the marker that is 3.0 or more standard deviations, e.g., 3.5 or more, 4.0 or more, 4.5 or more, or 5.0 or more standard deviations, higher than an average signal generated for the marker by a population of negative control cells.

Any existing, available or conventional separation, detection and/or quantification methods may be used to measure the presence or absence (e.g., readout being present vs. absent; or detectable amount vs. undetectable amount) and/or quantity (e.g., readout being an absolute or relative quantity) of CD70 positive cells in a biological sample from a subject. By means of an example, standard immunological assay methods may be employed, including without limitation immunohistochemistry, immunocytochemistry, flow cytometry, mass cytometry, fluorescence activated cell sorting (FACS), fluorescence microscopy, fluorescence based cell sorting using microfluidic systems, immunoaffinity adsorption based techniques such as affinity chromatography, magnetic particle separation, magnetic activated cell sorting or bead based cell sorting using microfluidic systems, enzyme-linked immunosorbent assay (ELISA) and ELISPOT based techniques, radioimmunoassay (RIA), Western blot, etc. Anti-CD70 antibodies suitable for use in such techniques are commercially available from a wide variety of vendors.

The terms “sample” or “biological sample” as used throughout this specification include any biological specimen obtained (isolated, removed) from a subject. Samples may include without limitation organ tissue (e.g., primary or metastatic tumor tissue), whole blood, plasma, serum, whole blood cells, red blood cells, white blood cells (e.g., peripheral blood mononuclear cells), saliva, urine, stool (feces), tears, sweat, sebum, nipple aspirate, ductal lavage, tumor exudates, synovial fluid, cerebrospinal fluid, lymph, fine needle aspirate, amniotic fluid, any other bodily fluid, exudate or secretory fluid, cell lysates, cellular secretion products, inflammation fluid, semen and vaginal secretions. Preferably, a sample may be readily obtainable by non-invasive or minimally invasive methods, such as blood collection (liquid biopsy'), urine collection, feces collection, tissue (e.g., tumor tissue) biopsy or fine-needle aspiration, allowing the provision/removal/isolation of the sample from a subject. The term “tissue” as used herein encompasses all types of cells of the human body including cells of organs but also including blood and other body fluids recited above. The tissue may be healthy or affected by pathological alterations, e.g., tumor tissue. The tissue may be from a living subject or may be cadaveric tissue. Particularly useful samples are those known to comprise, or expected or predicted to comprise, or known to potentially comprise, or expected or predicted to potentially comprise tumor cells and/or tumor microenvironment cells.

Any suitable weight or volume of a sample may be removed from a subject for analysis. Without limitation, a liquid sample may have a volume between 1 mL and 20 mL, e.g., 5 mL, 7.5 mL, 10 mL, 15 mL or 20 mL. A solid sample may have a weight of between 1 g and 20 g, e.g., 5 g, 7.5 g, 10 g, 15 g or 20 g.

In certain embodiments, the neoplastic disease such as cancer comprises 10% or more, 20% or more, 30% or more, 40% or more, 50% or more, 60% or more, 70% or more, 80% or more, 90% or more, or up to 100% CD70-positive cancerous cells, the percentage of CD70 positive cancerous cells expressed relative to all cancerous cell in a representative sample of the neoplastic disease lesion. In certain embodiments, the neoplastic disease such as cancer comprises less than 10% or more, such as 8% or less, 6% or less, 4% or less, 2% or less, or down to 0% CD70-positive cancerous cells, the percentage of CD70 positive cancerous cells expressed relative to all cancerous cell in a representative sample of the neoplastic disease lesion. Owing to the ability to target CD70-positive tumor microenvironment (TME) cells, such as cancer associated fibroblasts (CAFs), the engineered NK cells according to embodiments of the present invention can also be therapeutically effective in cancers in which the level of CD70 expression in the cancerous cells is comparatively low.

In certain embodiments, the neoplastic disease such as cancer comprises CD70-positive cancer associated fibroblasts (CAF). The phrases “cancer-associated fibroblasts”, “CAFs”, “tumor-associated fibroblast”, “carcinogenic associated fibroblast”, or “activated fibroblast” refer to a cell type within the tumor microenvironment that has been reported to promote tumorigenic features by initiating the remodelling of the extracellular matrix or by secreting cytokines. CAFs have been reported to be derived from normal fibroblasts, pericytes, smooth muscle cells, fibrocytes or mesenchymal stem cells. According to present knowledge, CAFs may support tumor growth by secreting growth factors, such as Vascular Endothelial Growth Factor (VEGF), Platelet Derived Growth Factor (PDGF) and Fibroblast Growth Factor (FGF), and other chemokines to stimulate angiogenesis, and thereby stimulate the growth of the tumor.

In certain embodiments, the neoplastic disease such as cancer comprises 10% or more, 20% or more, 30% or more, 40% or more, 50% or more, 60% or more, 70% or more, 80% or more, 90% or more, or up to 100% CD70-positive CAFs, the percentage of CD70 positive CAFs expressed relative to all CAFs in a representative sample of the neoplastic disease lesion.

In certain embodiments, the neoplastic disease such as cancer comprises CD70-positive cancerous cells and CD70-positive cancer associated fibroblasts (CAF).

In certain embodiments, the neoplastic disease such as cancer comprises: less than 10%, such as 8% or less, 6% or less, 4% or less, 2% or less, or down to 0% CD70-positive cancerous cells, the percentage of CD70 positive cancerous cells expressed relative to all cancerous cell in a representative sample of the neoplastic disease lesion; and 10% or more, such as 20% or more, 30% or more, 40% or more, 50% or more, 60% or more, 70% or more, 80% or more, 90% or more, or up to 100% CD70-positive CAFs, the percentage of CD70 positive CAFs expressed relative to all CAFs in a representative sample of the neoplastic disease lesion CD70-positive CAF.

In certain embodiments, the neoplastic disease is colorectal cancer (CC), more particularly in certain embodiments, colorectal cancer comprising CD70-positive CAFs. The inventors have characterised CC as frequently having only low number of CD70-positive cancerous cells (e.g., less than 10% or even less than 5%) but at the same time a comparatively sizeable proportion of CD70-positive CAFs. Moreover, the % of CD70-positive CAFs seems to increase with more advanced stage of CC. Hence, preferably the CC is stage T1, more preferably stage T2, even more preferably stage T3, most preferably stage T4.

In certain embodiments, the colorectal cancer comprises 10% or more, 20% or more, 30% or more, 40% or more, 50% or more, 60% or more, 70% or more, 80% or more, 90% or more, or up to 100% CD70-positive CAFs, the percentage of CD70 positive CAFs expressed relative to all CAFs in a representative sample of the colorectal cancer lesion.

In certain embodiments, engineered NK cells as taught herein may be administered as the sole pharmaceutical agent (active pharmaceutical ingredient) or in combination with one or more other pharmaceutical agents where the combination causes no unacceptable adverse effects. For example, the NK cells may be combined with known anti-cancer therapy or therapies, such as for example surgery, radiotherapy, chemotherapy, biological therapy, or combinations thereof. The term “chemotherapy” as used herein is conceived broadly and generally encompasses treatments using chemical substances or compositions. Chemotherapeutic agents may typically display cytotoxic or cytostatic effects. In certain embodiments, a chemotherapeutic agent may be an alkylating agent, a cytotoxic compound, an anti-metabolite, a plant alkaloid, a terpenoid, a topoisomerase inhibitor, or a combination thereof. The term “biological therapy” as used herein is conceived broadly and generally encompasses treatments using biological substances or compositions, such as biomolecules, or biological agents, such as viruses or cells. In certain embodiments, a biomolecule may be a peptide, polypeptide, protein, nucleic acid, or a small molecule (such as primary metabolite, secondary metabolite, or natural product), or a combination thereof. Examples of suitable biomolecules include without limitation interleukins, cytokines, anti-cytokines, tumor necrosis factor (TNF), cytokine receptors, vaccines, interferons, enzymes, therapeutic antibodies, antibody fragments, antibody-like protein scaffolds, or combinations thereof. Examples of suitable biomolecules include but are not limited to aldesleukine, alemtuzumab, atezolizumab, bevacizumab, blinatumomab, brentuximab vedotine, catumaxomab, cetuximab, daratumumab, denileukin diftitox, denosumab, dinutuximab, elotuzumab, gemtuzumab ozogamicin, ⁹⁰Y-ibritumomab tiuxetan, idarucizumab, interferon A, ipilimumab, necitumumab, nivolumab, obinutuzumab, ofatumumab, olaratumab, panitumumab, pembrolizumab, ramucirumab, rituximab, tasonermin, ¹³¹I-tositumomab, trastuzumab, Ado-trastuzumab emtansine, and combinations thereof Examples of suitable oncolytic viruses include but are not limited to talimogene laherparepvec. Further categories of anti-cancer therapy include inter alfa hormone therapy (endocrine therapy), immunotherapy, and stem cell therapy, which are commonly considered as subsumed within biological therapies. Examples of suitable hormone therapies include but are not limited to tamoxifen; aromatase inhibitors, such as atanastrozole, exemestane, letrozole, and combinations thereof; luteinizing hormone blockers such as goserelin, leuprorelin, triptorelin, and combinations thereof; anti-androgens, such as bicalutamide, cyproterone acetate, flutamide, and combinations thereof; gonadotrophin releasing hormone blockers, such as degarelix; progesterone treatments, such as medroxyprogesterone acetate, megestrol, and combinations thereof; and combinations thereof. The term “immunotherapy” broadly encompasses any treatment that modulates a subject's immune system. In particular, the term comprises any treatment that modulates an immune response, such as a humoral immune response, a cell-mediated immune response, or both. Immunotherapy comprises cell-based immunotherapy in which immune cells, such as T cells and/or dendritic cells, are transferred into the patient. The term also comprises an administration of substances or compositions, such as chemical compounds and/or biomolecules (e.g., antibodies, antigens, interleukins, cytokines, or combinations thereof), that modulate a subject's immune system. Examples of cancer immunotherapy include without limitation treatments employing monoclonal antibodies, for example Fc-engineered monoclonal antibodies against proteins expressed by tumor cells, immune checkpoint inhibitors, prophylactic or therapeutic cancer vaccines, adoptive cell therapy, and combinations thereof. Examples of immune checkpoint targets for inhibition include without limitation PD-1 (examples of PD-1 inhibitors include without limitation pembrolizumab, nivolumab, and combinations thereof), CTLA-4 (examples of CTLA-4 inhibitors include without limitation ipilimumab, tremelimumab, and combinations thereof), PD-L1 (examples of PD-L1 inhibitors include without limitation atezolizumab), LAG3, B7-H3 (CD276), B7-H4, TIM-3, BTLA, A2aR, killer cell immunoglobulin-like receptors (KIRs), IDO, and combinations thereof. Another approach to therapeutic anti-cancer vaccination includes dendritic cell vaccines. The term broadly encompasses vaccines comprising dendritic cells which are loaded with antigen(s) against which an immune reaction is desired. Adoptive cell therapy (ACT) can refer to the transfer of cells, most commonly immune-derived cells, such as in particular cytotoxic T cells (CTLs), back into the same patient or into a new recipient host with the goal of transferring the immunologic functionality and characteristics into the new host. If possible, use of autologous cells helps the recipient by minimizing tissue rejection and graft vs. host disease issues. Various strategies may for example be employed to genetically modify T cells by altering the specificity of the T cell receptor (TCR) for example by introducing new TCR α and β chains with selected peptide specificity. Alternatively, chimeric antigen receptors (CARs) may be used in order to generate immunoresponsive cells, such as T cells, specific for selected targets, such as malignant cells, with a wide variety of receptor chimera constructs having been described. Stem cell therapies in cancer commonly aim to replace bone marrow stem cells destroyed by radiation therapy and/or chemotherapy, and include without limitation autologous, syngeneic, or allogeneic stem cell transplantation. The stem cells, in particular hematopoietic stem cells, are typically obtained from bone marrow, peripheral blood or umbilical cord blood. Details of administration routes, doses, and treatment regimens of anti-cancer agents are known in the art, for example as described in “Cancer Clinical Pharmacology” (2005) ed. By Jan H. M. Schellens, Howard L. McLeod and David R. Newell, Oxford University Press. Active components of any combination therapy may be admixed or may be physically separated and may be administered simultaneously or sequentially in any order.

The present application also provides aspects and embodiments as set forth in the following Statements:
Statement 1. A natural killer (NK) cell engineered to express a chimeric antigen receptor (CAR), wherein the CAR comprises the extracellular domain of CD27 or a CD70-binding portion thereof.
Statement 2. The NK cell according to Statement 1, wherein the CAR further comprises the intramembrane domain of CD27.
Statement 3. The NK cell according to Statement 1 or 2, wherein the CAR lacks all or a portion of the intracellular domain of CD27.
Statement 4. The NK cell according to any one of Statements 1 to 3, wherein the CAR lacks all of the intracellular domain of CD27.
Statement 5. The NK cell according to any one of Statements 1 to 4, wherein the intracellular portion of the CAR comprises at least one intracellular activation domain.
Statement 6. The NK cell according to Statement 5, wherein the at least one intracellular activation domain is selected from the group consisting of a CD3ζ activation domain, a FcRγ activation domain, and combinations thereof.
Statement 7. The NK cell according to any one of Statements 1 to 6, wherein the intracellular portion of the CAR comprises at least one intracellular costimulatory domain.
Statement 8. The NK cell according to Statement 7, wherein the at least one intracellular costimulatory domain is selected from the group consisting of a CD28 costimulatory domain, a 4-1BB costimulatory domain, a DAP10 costimulatory domain, a OX40 costimulatory domain, an ICOS costimulatory domain, and combinations thereof.
Statement 9. The NK cell according to any one of Statements 5 to 8, wherein the CAR comprises the CD3ζ intracellular activation domain.
Statement 10. The NK cell according to any one of Statements 5 to 9, wherein the CAR comprises the 4-1BB intracellular co-stimulatory domain.
Statement 11. The NK cell according to any one of Statements 1 to 10, wherein the CAR:
   comprises the extracellular and intramembrane domains of CD27, the CD3ζ intracellular activation domain, and the 4-1BB intracellular co-stimulatory domain;
   consists essentially of the extracellular and intramembrane domains of CD27, the CD3ζ intracellular activation domain, and the 4-1BB intracellular co-stimulatory domain; or
   consists of the extracellular and intramembrane domains of CD27, the CD3ζ intracellular activation domain, and the 4-1BB intracellular co-stimulatory domain.
Statement 12. The NK cell according to any one of Statements 1 to 11, wherein the CAR:
   comprises an amino acid sequence at least 80% identical to SEQ ID NO: 1;
   consists essentially of an amino acid sequence at least 80% identical to SEQ ID NO: 1;
   consists of an amino acid sequence at least 80% identical to SEQ ID NO: 1;
   comprises an amino acid sequence at least 85% identical to SEQ ID NO: 1;
   consists essentially of an amino acid sequence at least 85% identical to SEQ ID NO: 1;
   consists of an amino acid sequence at least 85% identical to SEQ ID NO: 1;
   comprises an amino acid sequence at least 90% identical to SEQ ID NO: 1;
   consists essentially of an amino acid sequence at least 90% identical to SEQ ID NO: 1;
   consists of an amino acid sequence at least 90% identical to SEQ ID NO: 1;
   comprises an amino acid sequence at least 95% identical to SEQ ID NO: 1;
   consists essentially of an amino acid sequence at least 95% identical to SEQ ID NO: 1;
   consists of an amino acid sequence at least 95% identical to SEQ ID NO: 1;
   comprises an amino acid sequence at least 96%, at least 97%, at least 98% or at least 99% identical to SEQ ID NO: 1;
   consists essentially of an amino acid sequence at least 96%, at least 97%, at least 98% or at least 99% identical to SEQ ID NO: 1;
   consists of an amino acid sequence at least 96%, at least 97%, at least 98% or at least 99% identical to SEQ ID NO: 1;
   comprises an amino acid sequence set forth in SEQ ID NO: 1;
   consists essentially of an amino acid sequence set forth in SEQ ID NO: 1; or
   consists of an amino acid sequence set forth in SEQ ID NO: 1.
Statement 13. The NK cell according to any one of Statements 1 to 12, wherein the NK cell has been isolated from a subject.
Statement 14. The NK cell according to any one of Statements 1 to 13, wherein the NK cell has been isolated from cord blood or peripheral blood of a subject.
Statement 15. The NK cell according to Statements 13 or 14, wherein the NK cell has been optionally cultured in vitro.
Statement 16. The NK cell according to any one of Statements 1 to 13, wherein the NK cell has been in vitro differentiated from a hematopoietic stem cell or from an induced pluripotent stem (iPS) cell.
Statement 17. The NK cell according to any one of Statements 1 to 13, wherein the NK cell is from a clonal NK-cell line.
Statement 18. The NK cell according to any one of Statements 1 to 13, wherein the NK cell is an NK-92 cell.
Statement 19. The NK cell according to any one of Statements 1 to 18, wherein the NK cell is engineered to further express one or more immunostimulatory cytokines.
Statement 20. The NK cell according to Statement 19, wherein the one or more immunostimulatory cytokine is one or more immunostimulatory interleukins (IL).
Statement 21. The NK cell according to Statement 20, wherein the immunostimulatory interleukin is IL-15, IL-12, and/or IL-21, preferably human IL-15, IL-12, and/or IL-21, more preferably IL-15 and/or IL-21, even more preferably human IL-15 and/or IL-21, still more preferably IL-15, particularly preferably human IL-15.
Statement 22. The NK cell according to any one of Statements 1 to 21, wherein the expression of the CAR and of the optional one or more immunostimulatory cytokines is each independently constitutive or inducible.
Statement 23. A method for producing the NK cell according to any one of Statements 1 to 22 comprising introducing a nucleic acid encoding the CAR as defined in any one of Statements 1 to 22 in an expressible format, and optionally a nucleic acid encoding one or more immunostimulatory cytokine in an expressible format, into a starting population of NK cells.
Statement 24. The method according to Statement 23, wherein the nucleic acid or nucleic acids are introduced into the starting population of NK cells by electroporation.
Statement 25. The method according to Statement 23 or 24, wherein the nucleic acid or nucleic acids are mRNA.
Statement 26. The method according to any one of Statements 23 to 25, further comprising selecting and/or expanding NK cells which comprise said nucleic acid or nucleic acids and which are capable of expressing the CAR and optionally the one or more immunostimulatory cytokine.
Statement 27. A pharmaceutical composition comprising the NK cell as defined in any one of Statements 1 to 22 and a pharmaceutically acceptable carrier.

Statement 28. The NK cell according to any one of Statements 1 to 22 or the pharmaceutical composition according to Statement 27 for use in therapy.
Statement 29. The NK cell according to any one of Statements 1 to 22 or the pharmaceutical composition according to Statement 27 for use in a method of treating a neoplastic disease.
Statement 30. The NK cell according to any one of Statements 1 to 22 or the pharmaceutical composition according to Statement 27 for use in a method of treating cancer.
Statement 31. A method for treating a subject in need thereof, the method comprising administering to the subject a therapeutically effective amount of the NK cell according to any one of Statements 1 to 22 or the pharmaceutical composition according to Statement 27.
Statement 32. A method for treating a subject having a neoplastic disease, the method comprising administering to the subject a therapeutically effective amount of the NK cell according to any one of Statements 1 to 22 or the pharmaceutical composition according to Statement 27.
Statement 33. A method for treating a subject having cancer, the method comprising administering to the subject a therapeutically effective amount of the NK cell according to any one of Statements 1 to 22 or the pharmaceutical composition according to Statement 27.
Statement 34. The NK cell or the pharmaceutical composition for use according to Statement 29 or 30, or the method according to Statement 32 or 33, wherein the neoplastic disease such as cancer comprises CD70-positive cancerous cells.
Statement 35. The NK cell or the pharmaceutical composition for use according to Statement 34, or the method according to Statement 34, wherein the neoplastic disease such as cancer comprises 10% or more, 20% or more, 30% or more, 40% or more, 50% or more, 60% or more, 70% or more, 80% or more, 90% or more, or up to 100% CD70-positive cancerous cells, the percentage of CD70 positive cancerous cells expressed relative to all cancerous cell in a representative sample of the neoplastic disease lesion.
Statement 36. The NK cell or the pharmaceutical composition for use according to Statement 34, or the method according to Statement 34, wherein the neoplastic disease such as cancer comprises less than 10% or more, such as 8% or less, 6% or less, 4% or less, 2% or less, or down to 0% CD70-positive cancerous cells, the percentage of CD70 positive cancerous cells expressed relative to all cancerous cell in a representative sample of the neoplastic disease lesion.
Statement 37. The NK cell or the pharmaceutical composition for use according to any one of Statements 29, 30 or 34 to 36, or the method according to any one of Statements 32 to 36, wherein the neoplastic disease such as cancer comprises CD70-positive cancer associated fibroblasts (CAFs).
Statement 38. The NK cell or the pharmaceutical composition for use according to Statement 37, or the method according to Statement 37, wherein the neoplastic disease such as cancer comprises 10% or more, 20% or more, 30% or more, 40% or more, 50% or more, 60% or more, 70% or more, 80% or more, 90% or more, or up to 100% CD70-positive CAFs, the percentage of CD70 positive CAFs expressed relative to all CAFs in a representative sample of the neoplastic disease lesion.
Statement 39. The NK cell or the pharmaceutical composition for use according to Statement 29 or 30, or the method according to Statement 32 or 33, wherein the neoplastic disease such as cancer comprises CD70-positive cancerous cells and CD70-positive cancer associated fibroblasts (CAFs).
Statement 40. The NK cell or the pharmaceutical composition for use according to Statement 39, or the method according to Statement 39, wherein the neoplastic disease comprises: less than 10%, such as 8% or less, 6% or less, 4% or less, 2% or less, or down to 0% CD70-positive cancerous cells, the percentage of CD70 positive cancerous cells expressed relative to all cancerous cell in a representative sample of the neoplastic disease lesion; and 10% or more, such as 20% or more, 30% or more, 40% or more, 50% or more, 60% or more, 70% or more, 80% or more, 90% or more, or up to 100% CD70-positive CAFs, the percentage of CD70 positive CAFs expressed relative to all CAFs in a representative sample of the neoplastic disease lesion CD70-positive CAFs.
Statement 41. The NK cell or the pharmaceutical composition for use according to any one of Statements 29, 30 or 34 to 40, or the method according to any one of Statements 32 to 40, wherein the neoplastic disease is colorectal cancer.
Statement 42. The NK cell or the pharmaceutical composition for use according to Statement 41, or the method according to Statement 41, wherein the colorectal cancer comprises CD70-positive CAFs.
Statement 43. The NK cell or the pharmaceutical composition for use according to Statement 42, or the method according to Statement 42, wherein the colorectal cancer comprises 10% or more, 20% or more, 30% or more, 40% or more, 50% or more, 60% or more, 70% or more, 80% or more, 90% or more, or up to 100% CD70-positive CAFs, the percentage of CD70 positive CAFs expressed relative to all CAFs in a representative sample of the colorectal cancer lesion.

While the invention has been described in conjunction with specific embodiments thereof, it is evident that many alternatives, modifications, and variations will be apparent to those skilled in the art in light of the foregoing description. Accordingly, it is intended to embrace all such alternatives, modifications, and variations as follows in the spirit and scope of the appended claims.

The herein disclosed aspects and embodiments of the invention are further supported by the following non-limiting examples.

### EXAMPLES

### Cell Lines:

The NK-92 cell line (purchased from the German Collection of Microorganisms and Cell Cultures

GmbH (DSMZ, Leibniz Institute, Inhoffenstr. 7B, D-38124 Braunschweig, Germany, # ACC 488)) was cultured in α-Minimum Essential Medium (α-MEM, Life Technologies, #32561037) medium supplemented with 12.5% Fetal Bovine Serum (FBS, Life Technologies, #10270106), 12.5% horse serum (Life Technologies; #16050122), 1% Penicillin/Streptomycin (P/S, Life Technologies; #15140122), 2mM L-glutamine (Life Technologies; #25030024) and 150 U/mL recombinant interleukin-2 (IL-2, ImmunoTools, #11340028). Cells were grown in suspension and were maintained in exponential growth in 5% CO₂+95% air in a humidified incubator at 37° C.

Different CD70⁺ target cell lines were used: a Burkitt lymphoma cell line (Raji), a colorectal cancer cell line (LIM2099), a pancreatic ductal carcinoma cell line (PANC-1), and a pancreatic cancer-associated fibroblast (CAF) cell line (RLT-PSC).

The Raji cell line (purchased from DSMZ, # ACC319) and the LIM2099 cell line (purchased from Sigma-Aldrich, # CBA-0164) were cultured in Roswell Park Memorial Institute (RPMI, Life Technologies, #52400025) medium supplemented with 10% FBS, 1% P/S and 2 mM L-glutamine. The PANC-1 cell line was purchased from the ATCC (# CRL-1469) and cultured in Dulbecco Modified Eagle Medium (DMEM, Life Technologies, #10938025) supplemented with 10% FBS, 1% P/S and 2mM L-glutamine. The RLT-PSC cell line (kindly provided by Prof. M. Löhr and R. Jesenofsky, University of Heidelberg, Mannheim, Germany) was cultured in DMEM-F12 (Life Technologies, #11320074) supplemented with 10% FBS, 1% P/S and 2mM L-glutamine. Cells were grown as monolayers and were maintained in exponential growth in 5% CO₂+95% air in a humidified incubator at 37° C.

Generation of CD70-Directed CAR mRNA:

A CD27-CAR construct containing (1) the extracellular and transmembrane part of the CD27 receptor, (2) the 41BB costimulatory domain, (3) the CD3ζ intracellular T cell activation domain and (4) the IL-15 cytokine cassette (as shown in SEQ ID NO: 21, and encoded by SEQ ID NO: 31), was developed using the CellRapeutics™ Chimeric Antigen Receptor Technology platform from Creative Biolabs (Shirley, NY, USA). To generate a construct with and without IL-15 the IL-15 cytokine cassette was cut out using the EcoRI restriction enzyme (Life Technologies, #ER0271).

The CD27-CAR construct containing plasmids (with or without an IL-15 cytokine cassette) were amplified in Escherichia coli bacteria, purified with the NucleoBond Xtra Midi Plus EF kit (Macherey Nagel; #740422.50) and linearized using the compatible Pmel restriction enzyme (Life Technologies; #ER1342). CD27-CAR messenger RNA (mRNA) was generated starting from 1 μg linearized CD27-CAR DNA using the mMESSAGE mMACHINE™ T7 Transcription Kit (Life Technologies, #AM1344) and stored at −80° C. for further usage.

### Development of CD70-Directed CAR-NK-92 Cells:

To optimize electroporation efficiency NK-92 cells were stimulated with 150 U/mL IL-2 24 hours before electroporation, and washed and resuspended in OptiMEM™ medium (Life Technologies, #11058021) right before electroporation. Per condition 5-20×10⁶ NK-92 cells were electroporated in 20 μL OptiMEM medium with 20 μg CAR mRNA using the GenePulser (Bio-Rad, Hercules, CA, USA) applying the time constant protocol with following settings: 300 V, 12 ms and cuvette 4. NK-92 cells electroporated without mRNA (MOCK) were used as negative control. After electroporation the cells were resuspended in α-MEM medium without IL-2 as recovery medium.

### Validation of CD70-Directed CAR-NK-92 Cells:

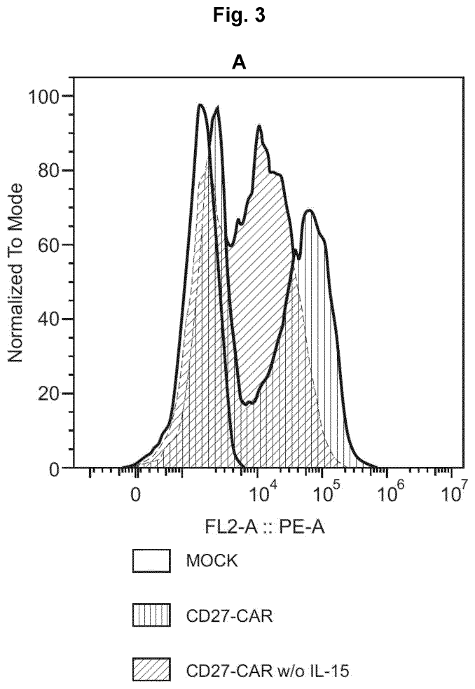

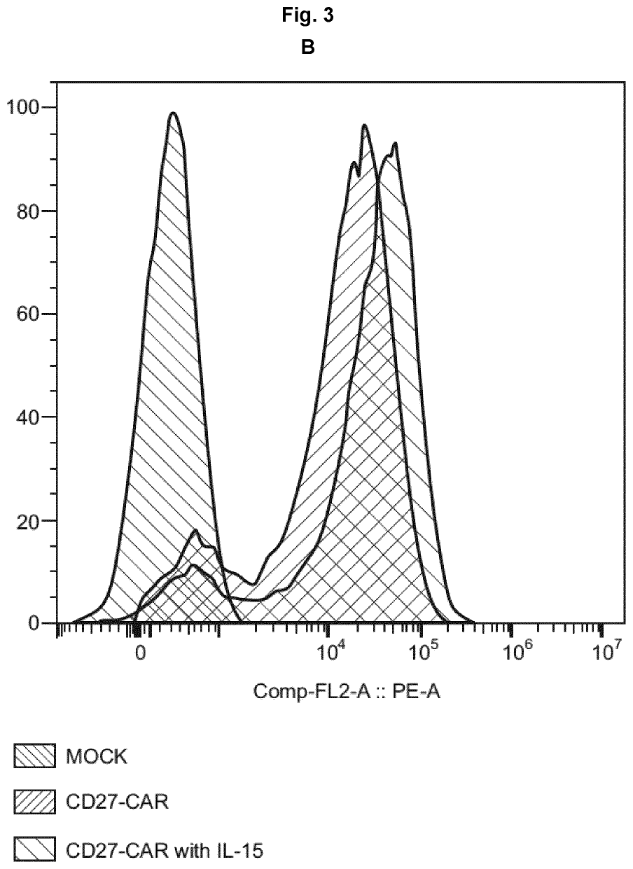

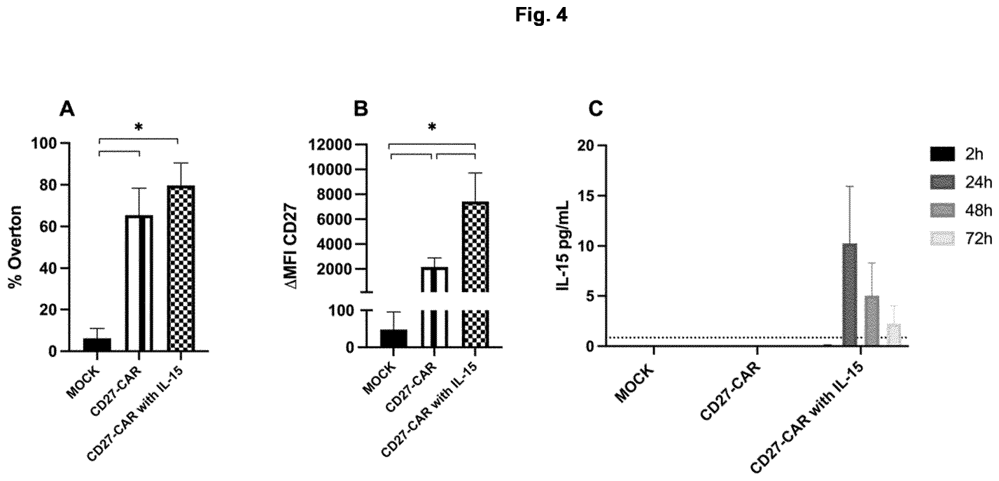

CD27-CAR expression (expression of the extracellular part of the CAR construct, CD27) was determined on the CytoFLEX flow cytometer (Beckman Coulter, Brea, CA, USA) 24 hours after electroporation using the monoclonal PE-conjugated anti-human CD27 antibody (Cell Signaling Technology, Danvers, CA, USA; clone 0323; #55584S) and the corresponding IgG1 isotype control (Cell Signaling Technology; clone MOPC-21; #63630). The 7-AAD (BioLegend, #420403) fluorescent intercalating viability dye staining was used to analyse CD27 expression on live cells. MOCK electroporated NK-92 cells were used as negative control sample. Results are shown in FIGS. 3 and 4.

Validation of the production and secretion of the IL-15 cytokine into the supernatants of the cultures was performed using multiplex electrochemiluminescence (Meso Scale Discovery Inc., Rockville, USA, #K151URK-1).

### In Vitro Cytotoxic Potential of the CD70-Directed CAR-NK-92 Cells:

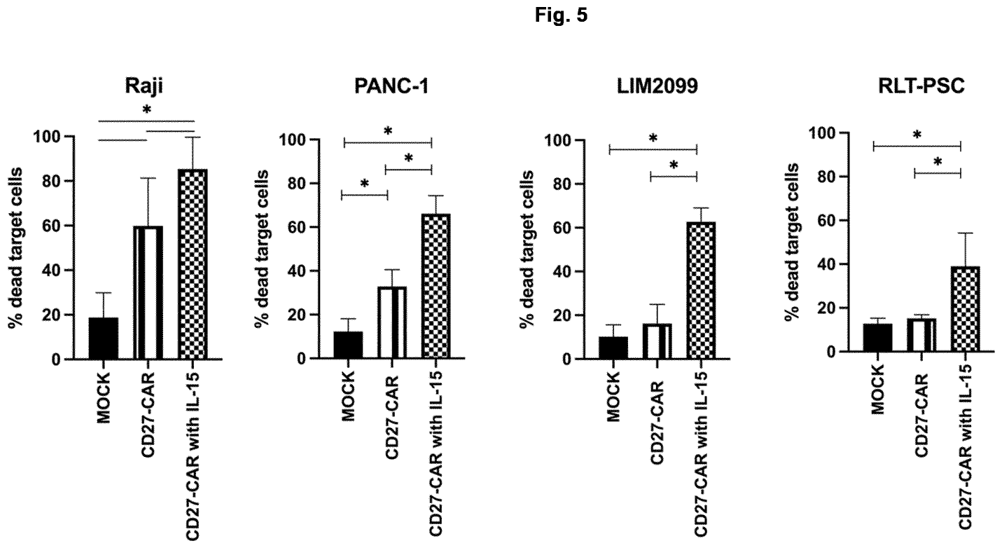

The in vitro killing capacity of the CD70-directed CAR-NK-92 cells was assessed by coculturing the different effector conditions (MOCK, CD27-CAR and CD27-CAR with the IL-15 cytokine cassette, 24 hours after electroporation) together with different CD70⁺ target cell lines: a Burkitt lymphoma cell line (Raji), a colorectal cancer and pancreatic cancer cell line (LIM2099 and PANC-1, respectively) and a pancreatic CAF cell line (RLT-PSC), in a 5:1 effector:target ratio for 4 hours in sterile FACS tubes. After 4 hours of co-culture, cell death of the target cell lines was measured on the CytoFLEX flow cytometer by staining for the viability intercalating fluorescent dye 7-AAD and the apoptotic cell death marker Annexin V (BD Bioscience, #51-65875X). To differentiate effector and target cells, the latter was transiently labeled with a green fluorescent dye PKH67 (Sigma-Aldrich, #MIDI67-KT). Results are show in FIG. 5.

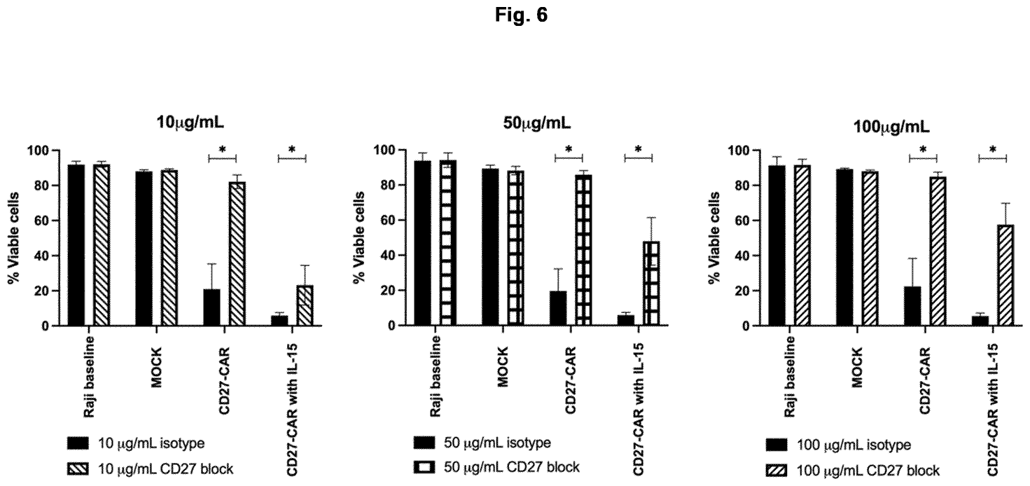

In order to analyze if the observed killing by the CD70-CAR-NK-92 cells is CAR-specific, the extracellular antigen-recognition domain, CD27, was blocked during coculture with the CD70⁺ Raji cell line. The different effector conditions (MOCK, CD27-CAR and CD27-CAR with the IL-15 cytokine cassette) were 6 hours after electroporation incubated overnight with a monoclonal neutralizing anti-CD27 antibody (R&D Systems; #MAB382) and the corresponding IgG1 isotype control (R&D Systems; #MAB002) in three different concentrations (10 μg/mL, 50 μg/mL and 100 μg/mL). These effector cells were cocultured with the Raji cell line 24 hours after electroporation for 4 hours in a 5:1 effector:target ratio in sterile FACS tubes. Raji cells incubated with neutralizing antibody and isotype were used as control condition. Detection of the amount of target cell death was performed as previously explained. Results are show in FIG. 6.

### In Vitro Cytotoxic Potential of the CD70-Directed CAR-NK-92 Cells and Further Cytokines:

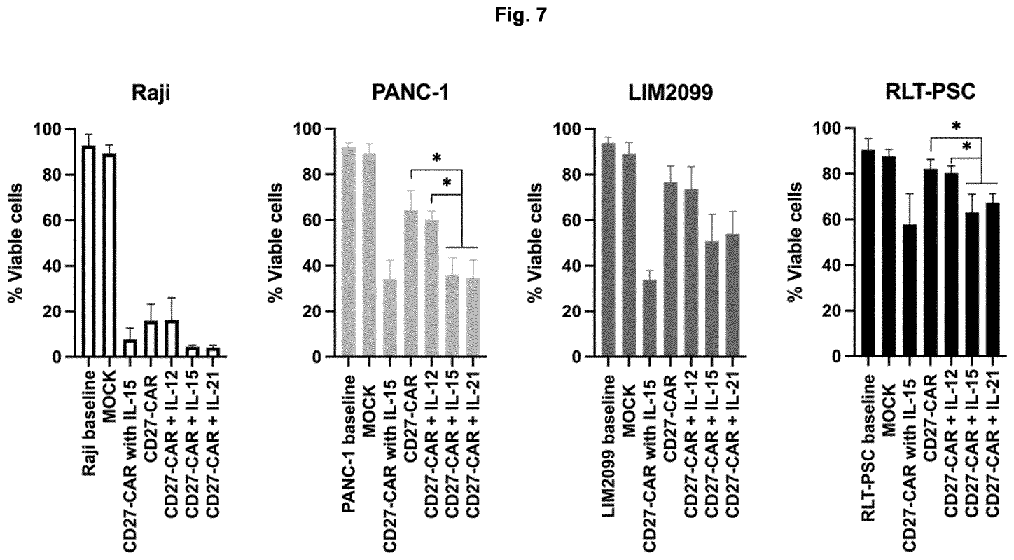

The capacity of the IL-12, IL-15 and IL-21 cytokines in improving the cytotoxic potential of the CD70-directed CAR-NK-92 cells was determined. The CD27-CAR without the IL-15 cytokine cassette was 6 hours after electroporation incubated overnight with an effector dose 50 (ED50; provided on R&D Systems) of the recombinant IL-12 (R&D Systems, #219-IL-005, 0.05 ng/mL), the recombinant IL-15 (R&D Systems, #247-ILB-005, 2.60 ng/mL) and the recombinant IL-21 (R&D Systems, #8879-ILB-010, 8 ng/mL) cytokines, to mimic or emulate the effect of the respective cytokines when co-expressed with the CAR by the NK-92 cells. The three effector conditions (MOCK, CD27-CAR and CD27-CAR with the IL-15 cytokine cassette) and the stimulated CD27-CAR-92 cells were cocultured 24 hours after electroporation together with the different CD70⁺ target cell lines (Raji, LIM2099, PANC-1 and RLT-PSC) in a 5:1 effector:target ratio for 4 hours in sterile FACS tubes. Detection of the amount of target cell death was performed as previously explained. Results are show in FIG. 7.

### Statistical Analysis:

Prism 9.1.2 software (GraphPad) was used for data comparison, graphical data representations and statistical computations. The Kruskal-Wallis test was used to compare means between more than two groups. The Mann-Whitney U test was used to compare means between two groups. All statistical analyses were performed on a minimum of three independent experiments. p-value≤0.05 was considered statistically significant.

1. A natural killer (NK) cell engineered to express a chimeric antigen receptor (CAR), wherein the CAR comprises the extracellular domain of CD27 or a CD70-binding portion thereof.

2. The NK cell according to claim 1, wherein the CAR further comprises the intramembrane domain of CD27 and/or lacks all or a portion of the intracellular domain of CD27, preferably lacks all of the intracellular domain of CD27.

3. The NK cell according to claim 1 or 2, wherein the intracellular portion of the CAR comprises at least one intracellular activation domain, such as a CD3ζ or FcRγ intracellular activation domain, and optionally and preferably at least one intracellular costimulatory domain, such as a CD28, 4-1BB, DAP10, OX40 and/or ICOS intracellular costimulatory domain.

4. The NK cell according to claim 3, wherein the CAR comprises the CD3ζ intracellular activation domain and/or the 4-1BB intracellular co-stimulatory domain.

5. The NK cell according to any one of claims 1 to 4, wherein the CAR comprises, consists essentially of or consists of the extracellular and intramembrane domains of CD27, the CD3ζ intracellular activation domain, and the 4-1BB intracellular co-stimulatory domain.

6. The NK cell according to any one of claims 1 to 5, wherein the CAR comprises, consists essentially of or consists of an amino acid sequence at least 80% identical, preferably at least 90% identical, more preferably at least 95% identical, particularly preferably 100% identical to SEQ ID NO: 1.

7. The NK cell according to any one of claims 1 to 6, wherein the NK cell has been isolated from a subject, such as from cord blood or peripheral blood of a subject, and has been optionally cultured in vitro; or the NK cell has been in vitro differentiated from a hematopoietic stem cell or from an induced pluripotent stem (iPS) cell; or preferably the NK cell is from a clonal NK-cell line, such as particularly preferably is an NK-92 cell.

8. The NK cell according to any one of claims 1 to 7, wherein the NK cell is engineered to further express one or more immunostimulatory cytokine, such as one or more immunostimulatory interleukin (IL), preferably IL-15, IL-12 and/or IL-21, more preferably IL-15 and/or IL-21, particularly preferably human IL-15.

9. The NK cell according to any one of claims 1 to 8, wherein the expression of the CAR and of the optional one or more immunostimulatory cytokine is each independently constitutive or inducible.

10. A method for producing the NK cell according to any one of claims 1 to 9 comprising introducing a nucleic acid encoding the CAR as defined in any one of claims 1 to 9 in an expressible format, and optionally a nucleic acid encoding one or more immunostimulatory cytokine in an expressible format, into a starting population of NK cells, such as optionally by electroporation; and optionally selecting and/or expanding NK cells which comprise said nucleic acid or nucleic acids and which are capable of expressing the CAR and optionally the one or more immunostimulatory cytokine.

11. A pharmaceutical composition comprising the NK cell as defined in any one of claims 1 to 9 and a pharmaceutically acceptable carrier.

12. The NK cell according to any one of claims 1 to 9 or the pharmaceutical composition according to claim 11 for use in therapy.

13. The NK cell according to any one of claims 1 to 9 or the pharmaceutical composition according to claim 11 for use in a method of treating a neoplastic disease, particularly cancer.

14. The NK cell or the pharmaceutical composition for use according to claim 13, wherein the neoplastic disease comprises CD70-positive cancerous cells, such as 10% or more CD70-positive cancerous cells, and/or CD70-positive cancer associated fibroblasts (CAF), such as 10% or more CD70-positive CAF; such as wherein the neoplastic disease comprises less than 10% CD70-positive cancerous cells and 10% or more CD70-positive CAF.

15. The NK cell or the pharmaceutical composition for use according to claim 12 or 13, wherein the neoplastic disease is colorectal cancer comprising CD70-positive CAF.


SEQUENCE LISTING (35 sequences)

SEQ ID NO: 1 - protein, 347 aa, Artificial Sequence
  CD27-CAR polypeptide
ATPAPKSCPERHYWAQGKLCCQMCEPGTFLVKDCDQHRKAAQCDPCIPGVSFSPDHHTRPHCESCRHCNSGLLVRNCTITANAECACRNGWQCRDKECTECDPLPNPSLTARSSQALSPHPQPTHLPYVSEMLEARTAGHMQTLADFRQLPARTLSTHWPPQRSLCSSDFIRILVIFSGMFLVFTLAGALFLHKRGRKKLLYIFKQPFMRPVQTTQEEDGCSCRFPEEEEGGCELRVKFSRSADAPAYKQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 2 - protein, 260 aa, Homo sapiens
MARPHPWWLCVLGTLVGLSATPAPKSCPERHYWAQGKLCCQMCEPGTFLVKDCDQHRKAAQCDPCIPGVSFSPDHHTRPHCESCRHCNSGLLVRNCTITANAECACRNGWQCRDKECTECDPLPNPSLTARSSQALSPHPQPTHLPYVSEMLEARTAGHMQTLADFRQLPARTLSTHWPPQRSLCSSDFIRILVIFSGMFLVFTLAGALFLHQRRKYRSNKGESPVEPAEPCHYSCPREEEGSTIPIQEDYRKPEPACSP

SEQ ID NO: 3 - protein, 172 aa, Homo sapiens
ATPAPKSCPERHYWAQGKLCCQMCEPGTFLVKDCDQHRKAAQCDPCIPGVSFSPDHHTRPHCESCRHCNSGLLVRNCTITANAECACRNGWQCRDKECTECDPLPNPSLTARSSQALSPHPQPTHLPYVSEMLEARTAGHMQTLADFRQLPARTLSTHWPPQRSLCSSDFIR

SEQ ID NO: 4 - protein, 21 aa, Homo sapiens
ILVIFSGMFLVFTLAGALFLH

SEQ ID NO: 5 - protein, 48 aa, Homo sapiens
QRRKYRSNKGESPVEPAEPCHYSCPREEEGSTIPIQEDYRKPEPACSP

SEQ ID NO: 6 - protein, 112 aa, Homo sapiens
RVKFSRSADAPAYKQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 7 - protein, 42 aa, Homo sapiens
KRGRKKLLYIFKQPFMRPVQTTQEEDGCSCRFPEEEEGGCEL

SEQ ID NO: 8 - protein, 19 aa, Homo sapiens
MARPHPWWLCVLGTLVGLS

SEQ ID NO: 9 - protein, 366 aa, Artificial Sequence
  CD27-CAR polypeptide
MARPHPWWLCVLGTLVGLSATPAPKSCPERHYWAQGKLCCQMCEPGTFLVKDCDQHRKAAQCDPCIPGVSFSPDHHTRPHCESCRHCNSGLLVRNCTITANAECACRNGWQCRDKECTECDPLPNPSLTARSSQALSPHPQPTHLPYVSEMLEARTAGHMQTLADFRQLPARTLSTHWPPQRSLCSSDFIRILVIFSGMFLVFTLAGALFLHKRGRKKLLYIFKQPFMRPVQTTQEEDGCSCRFPEEEEGGCELRVKFSRSADAPAYKQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 10 - protein, 193 aa, Homo sapiens
ATPAPKSCPERHYWAQGKLCCQMCEPGTFLVKDCDQHRKAAQCDPCIPGVSFSPDHHTRPHCESCRHCNSGLLVRNCTITANAECACRNGWQCRDKECTECDPLPNPSLTARSSQALSPHPQPTHLPYVSEMLEARTAGHMQTLADFRQLPARTLSTHWPPQRSLCSSDFIRILVIFSGMFLVFTLAGALFLH

SEQ ID NO: 11 - DNA, 579 bp, Homo sapiens
gctacaccagctcctaagagctgccccgagagacactattgggcccagggcaagctgtgctgccagatgtgtgaacctggcaccttcctggtcaaggactgcgaccagcacagaaaggccgctcagtgcgatccttgtatccccggcgtgtccttctctcccgaccaccacacaagacctcactgcgagagctgcagacactgcaattctggactgctcgtgcggaactgcaccatcacagccaatgccgagtgcgcctgcagaaatggctggcagtgccgggacaaagaatgtaccgagtgcgaccctctgcctaatcctagcctgaccgccagaagcagccaggctttgtctcctcatcctcagcctacacatctgccctacgtgtccgagatgctggaagccagaacagccggccatatgcagaccctggccgactttagacagctgcccgccagaacactgagcacccattggcctccacagagaagcctgtgcagcagcgacttcatccggatcctcgtgatcttcagcggcatgttcctggtgttcacactggctggcgccctgtttctgcat

SEQ ID NO: 12 - DNA, 336 bp, Homo sapiens
cgcgtgaaattcagccgcagcgcagatgctccagcctacaagcaggggcagaaccagctctacaacgaactcaatcttggtcggagagaggagtacgacgtgctggacaagcggagaggacgggacccagaaatgggcgggaagccgcgcagaaagaatccccaagagggcctgtacaacgagctccaaaaggataagatggcagaagcctatagcgagattggtatgaaaggggaacgcagaagaggcaaaggccacgacggactgtaccagggactcagcaccgccaccaaggacacctatgacgctcttcacatgcaggccctgccgcctcgg

SEQ ID NO: 13 - DNA, 126 bp, Homo sapiens
aagcgcggtcggaagaagctgctgtacatctttaagcaacccttcatgaggcctgtgcagactactcaagaggaggacggctgttcatgccggttcccagaggaggaggaaggcggctgcgaactg

SEQ ID NO: 14 - DNA, 1098 bp, Artificial Sequence
  CD27-CAR construct
atggccagacctcatccttggtggctgtgtgtgctgggcacactcgttggcctgtctgctacaccagctcctaagagctgccccgagagacactattgggcccagggcaagctgtgctgccagatgtgtgaacctggcaccttcctggtcaaggactgcgaccagcacagaaaggccgctcagtgcgatccttgtatccccggcgtgtccttctctcccgaccaccacacaagacctcactgcgagagctgcagacactgcaattctggactgctcgtgcggaactgcaccatcacagccaatgccgagtgcgcctgcagaaatggctggcagtgccgggacaaagaatgtaccgagtgcgaccctctgcctaatcctagcctgaccgccagaagcagccaggctttgtctcctcatcctcagcctacacatctgccctacgtgtccgagatgctggaagccagaacagccggccatatgcagaccctggccgactttagacagctgcccgccagaacactgagcacccattggcctccacagagaagcctgtgcagcagcgacttcatccggatcctcgtgatcttcagcggcatgttcctggtgttcacactggctggcgccctgtttctgcataagcgcggtcggaagaagctgctgtacatctttaagcaacccttcatgaggcctgtgcagactactcaagaggaggacggctgttcatgccggttcccagaggaggaggaaggcggctgcgaactgcgcgtgaaattcagccgcagcgcagatgctccagcctacaagcaggggcagaaccagctctacaacgaactcaatcttggtcggagagaggagtacgacgtgctggacaagcggagaggacgggacccagaaatgggcgggaagccgcgcagaaagaatccccaagagggcctgtacaacgagctccaaaaggataagatggcagaagcctatagcgagattggtatgaaaggggaacgcagaagaggcaaaggccacgacggactgtaccagggactcagcaccgccaccaaggacacctatgacgctcttcacatgcaggccctgccgcctcgg

SEQ ID NO: 15 - protein, 162 aa, Homo sapiens
MRISKPHLRSISIQCYLCLLLNSHFLTEAGIHVFILGCFSAGLPKTEANWVNVISDLKKIEDLIQSMHIDATLYTESDVHPSCKVTAMKCFLLELQVISLESGDASIHDTVENLIILANNSLSSNGNVTESGCKECEELEEKNIKEFLQSFVHIVQMFINTS

SEQ ID NO: 16 - DNA, 486 bp, Homo sapiens
atgagaatcagcaagccccacctgagatccatcagcatccagtgctacctgtgcctgctgctgaacagccactttctgacagaggccggcatccacgtgttcatcctgggctgtttttctgccggcctgcctaagaccgaggccaactgggttaacgtgatcagcgacctgaagaagatcgaggacctgatccagagcatgcacatcgacgccacactgtacaccgagagcgacgtgcaccctagctgtaaagtgaccgccatgaagtgctttctgctggaactgcaagtgatcagcctggaaagcggcgacgccagcatccacgacaccgtggaaaacctgatcatcctggccaacaacagcctgagcagcaacggcaatgtgaccgagtccggctgcaaagagtgcgaggaactggaagagaagaatatcaaagagttcctgcagagcttcgtgcacatcgtgcagatgttcatcaacaccagc

SEQ ID NO: 17 - protein, 18 aa, Artificial Sequence
  T2A peptide
EGRGSLLTCGDVEENPGP

SEQ ID NO: 18 - protein, 19 aa, Artificial Sequence
  P2A peptide
ATNFSLLKQAGDVEENPGP

SEQ ID NO: 19 - protein, 20 aa, Artificial Sequence
  E2A peptide
QCTNYALLKLAGDVESNPGP

SEQ ID NO: 20 - protein, 22 aa, Artificial Sequence
  F2A peptide
VKQTLNFDLLKLAGDVESNPGP

SEQ ID NO: 21 - protein, 554 aa, Artificial Sequence
  CAR-P2A-IL15 polypeptide
MARPHPWWLCVLGTLVGLSATPAPKSCPERHYWAQGKLCCQMCEPGTFLVKDCDQHRKAAQCDPCIPGVSFSPDHHTRPHCESCRHCNSGLLVRNCTITANAECACRNGWQCRDKECTECDPLPNPSLTARSSQALSPHPQPTHLPYVSEMLEARTAGHMQTLADFRQLPARTLSTHWPPQRSLCSSDFIRILVIFSGMFLVFTLAGALFLHKRGRKKLLYIFKQPFMRPVQTTQEEDGCSCRFPEEEEGGCELRVKFSRSADAPAYKQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPREFGSGATNFSLLKQAGDVEENPGPMRISKPHLRSISIQCYLCLLLNSHFLTEAGIHVFILGCFSAGLPKTEANWVNVISDLKKIEDLIQSMHIDATLYTESDVHPSCKVTAMKCFLLELQVISLESGDASIHDTVENLIILANNSLSSNGNVTESGCKECEELEEKNIKEFLQSFVHIVQMFINTSEF

SEQ ID NO: 22 - DNA, 1683 bp, Artificial Sequence
  CAR-P2A-IL15 construct
actagtgccaccatggccagacctcatccttggtggctgtgtgtgctgggcacactcgttggcctgtctgctacaccagctcctaagagctgccccgagagacactattgggcccagggcaagctgtgctgccagatgtgtgaacctggcaccttcctggtcaaggactgcgaccagcacagaaaggccgctcagtgcgatccttgtatccccggcgtgtccttctctcccgaccaccacacaagacctcactgcgagagctgcagacactgcaattctggactgctcgtgcggaactgcaccatcacagccaatgccgagtgcgcctgcagaaatggctggcagtgccgggacaaagaatgtaccgagtgcgaccctctgcctaatcctagcctgaccgccagaagcagccaggctttgtctcctcatcctcagcctacacatctgccctacgtgtccgagatgctggaagccagaacagccggccatatgcagaccctggccgactttagacagctgcccgccagaacactgagcacccattggcctccacagagaagcctgtgcagcagcgacttcatccggatcctcgtgatcttcagcggcatgttcctggtgttcacactggctggcgccctgtttctgcataagcgcggtcggaagaagctgctgtacatctttaagcaacccttcatgaggcctgtgcagactactcaagaggaggacggctgttcatgccggttcccagaggaggaggaaggcggctgcgaactgcgcgtgaaattcagccgcagcgcagatgctccagcctacaagcaggggcagaaccagctctacaacgaactcaatcttggtcggagagaggagtacgacgtgctggacaagcggagaggacgggacccagaaatgggcgggaagccgcgcagaaagaatccccaagagggcctgtacaacgagctccaaaaggataagatggcagaagcctatagcgagattggtatgaaaggggaacgcagaagaggcaaaggccacgacggactgtaccagggactcagcaccgccaccaaggacacctatgacgctcttcacatgcaggccctgccgcctcgggaattcggttccggagccacgaacttctctctgttaaagcaagcaggagacgtggaagaaaaccccggtcccatgagaatcagcaagccccacctgagatccatcagcatccagtgctacctgtgcctgctgctgaacagccactttctgacagaggccggcatccacgtgttcatcctgggctgtttttctgccggcctgcctaagaccgaggccaactgggttaacgtgatcagcgacctgaagaagatcgaggacctgatccagagcatgcacatcgacgccacactgtacaccgagagcgacgtgcaccctagctgtaaagtgaccgccatgaagtgctttctgctggaactgcaagtgatcagcctggaaagcggcgacgccagcatccacgacaccgtggaaaacctgatcatcctggccaacaacagcctgagcagcaacggcaatgtgaccgagtccggctgcaaagagtgcgaggaactggaagagaagaatatcaaagagttcctgcagagcttcgtgcacatcgtgcagatgttcatcaacaccagcgaattctaactcgag

SEQ ID NO: 23 - protein, 162 aa, Homo sapiens
MRSSPGNMERIVICLMVIFLGTLVHKSSSQGQDRHMIRMRQLIDIVDQLKNYVNDLVPEFLPAPEDVETNCEWSAFSCFQKAQLKSANTGNNERIINVSIKKLKRKPPSTNAGRRQKHRLTCPSCDSYEKKPPKEFLERFKSLLQKMIHQHLSSRTHGSEDS

SEQ ID NO: 24 - protein, 153 aa, Homo sapiens
MRSSPGNMERIVICLMVIFLGTLVHKSSSQGQDRHMIRMRQLIDIVDQLKNYVNDLVPEFLPAPEDVETNCEWSAFSCFQKAQLKSANTGNNERIINVSIKKLKRKPPSTNAGRRQKHRLTCPSCDSYEKKPPKEFLERFKSLLQKVSTLSFI

SEQ ID NO: 25 - DNA, 486 bp, Homo sapiens
atgagatccagtcctggcaacatggagaggattgtcatctgtctgatggtcatcttcttggggacactggtccacaaatcaagctcccaaggtcaagatcgccacatgattagaatgcgtcaacttatagatattgttgatcagctgaaaaattatgtgaatgacttggtccctgaatttctgccagctccagaagatgtagagacaaactgtgagtggtcagctttttcctgctttcagaaggcccaactaaagtcagcaaatacaggaaacaatgaaaggataatcaatgtatcaattaaaaagctgaagaggaaaccaccttccacaaatgcagggagaagacagaaacacagactaacatgcccttcatgtgattcttatgagaaaaaaccacccaaagaattcctagaaagattcaaatcacttctccaaaagatgattcatcagcatctgtcctctagaacacacggaagtgaagattcc

SEQ ID NO: 26 - protein, 253 aa, Homo sapiens
MWPPGSASQPPPSPAAATGLHPAARPVSLQCRLSMCPARSLLLVATLVLLDHLSLARNLPVATPDPGMFPCLHHSQNLLRAVSNMLQKARQTLEFYPCTSEEIDHEDITKDKTSTVEACLPLELTKNESCLNSRETSFITNGSCLASRKTSFMMALCLSSIYEDLKMYQVEFKTMNAKLLMDPKRQIFLDQNMLAVIDELMQALNFNSETVPQKSSLEEPDFYKTKIKLCILLHAFRIRAVTIDRVMSYLNAS

SEQ ID NO: 27 - protein, 219 aa, Homo sapiens
MCPARSLLLVATLVLLDHLSLARNLPVATPDPGMFPCLHHSQNLLRAVSNMLQKARQTLEFYPCTSEEIDHEDITKDKTSTVEACLPLELTKNESCLNSRETSFITNGSCLASRKTSFMMALCLSSIYEDLKMYQVEFKTMNAKLLMDPKRQIFLDQNMLAVIDELMQALNFNSETVPQKSSLEEPDFYKTKIKLCILLHAFRIRAVTIDRVMSYLNAS

SEQ ID NO: 28 - protein, 328 aa, Homo sapiens
MCHQQLVISWFSLVFLASPLVAIWELKKDVYVVELDWYPDAPGEMVVLTCDTPEEDGITWTLDQSSEVLGSGKTLTIQVKEFGDAGQYTCHKGGEVLSHSLLLLHKKEDGIWSTDILKDQKEPKNKTFLRCEAKNYSGRFTCWWLTTISTDLTFSVKSSRGSSDPQGVTCGAATLSAERVRGDNKEYEYSVECQEDSACPAAEESLPIEVMVDAVHKLKYENYTSSFFIRDIIKPDPPKNLQLKPLKNSRQVEVSWEYPDTWSTPHSYFSLTFCVQVQGKSKREKKDRVFTDKTSATVICRKNASISVRAQDRYYSSSWSEWASVPCS

SEQ ID NO: 29 - DNA, 657 bp, Homo sapiens
atgtgtccagcgcgcagcctcctccttgtggctaccctggtcctcctggaccacctcagtttggccagaaacctccccgtggccactccagacccaggaatgttcccatgccttcaccactcccaaaacctgctgagggccgtcagcaacatgctccagaaggccagacaaactctagaattttacccttgcacttctgaagagattgatcatgaagatatcacaaaagataaaaccagcacagtggaggcctgtttaccattggaattaaccaagaatgagagttgcctaaattccagagagacctctttcataactaatgggagttgcctggcctccagaaagacctcttttatgatggccctgtgccttagtagtatttatgaagacttgaagatgtaccaggtggagttcaagaccatgaatgcaaagcttctgatggatcctaagaggcagatctttctagatcaaaacatgctggcagttattgatgagctgatgcaggccctgaatttcaacagtgagactgtgccacaaaaatcctcccttgaagaaccggatttttataaaactaaaatcaagctctgcatacttcttcatgctttcagaattcgggcagtgactattgatagagtgatgagctatctgaatgcttcc

SEQ ID NO: 30 - DNA, 984 bp, Homo sapiens
atgtgtcaccagcagttggtcatctcttggttttccctggtttttctggcatctcccctcgtggccatatgggaactgaagaaagatgtttatgtcgtagaattggattggtatccggatgcccctggagaaatggtggtcctcacctgtgacacccctgaagaagatggtatcacctggaccttggaccagagcagtgaggtcttaggctctggcaaaaccctgaccatccaagtcaaagagtttggagatgctggccagtacacctgtcacaaaggaggcgaggttctaagccattcgctcctgctgcttcacaaaaaggaagatggaatttggtccactgatattttaaaggaccagaaagaacccaaaaataagacctttctaagatgcgaggccaagaattattctggacgtttcacctgctggtggctgacgacaatcagtactgatttgacattcagtgtcaaaagcagcagaggctcttctgacccccaaggggtgacgtgcggagctgctacactctctgcagagagagtcagaggggacaacaaggagtatgagtactcagtggagtgccaggaggacagtgcctgcccagctgctgaggagagtctgcccattgaggtcatggtggatgccgttcacaagctcaagtatgaaaactacaccagcagcttcttcatcagggacatcatcaaacctgacccacccaagaacttgcagctgaagccattaaagaattctcggcaggtggaggtcagctgggagtaccctgacacctggagtactccacattcctacttctccctgacattctgcgttcaggtccagggcaagagcaagagagaaaagaaagatagagtcttcacggacaagacctcagccacggtcatctgccgcaaaaatgccagcattagcgtgcgggcccaggaccgctactatagctcatcttggagcgaatgggcatctgtgccctgcagt

SEQ ID NO: 31 - DNA, 1683 bp, Artificial Sequence
  CAR-p2A-IL15 construct
actagtgccaccatggccagacctcatccttggtggctgtgtgtgctgggcacactcgttggcctgtctgctacaccagctcctaagagctgccccgagagacactattgggcccagggcaagctgtgctgccagatgtgtgaacctggcaccttcctggtcaaggactgcgaccagcacagaaaggccgctcagtgcgatccttgtatccccggcgtgtccttctctcccgaccaccacacaagacctcactgcgagagctgcagacactgcaattctggactgctcgtgcggaactgcaccatcacagccaatgccgagtgcgcctgcagaaatggctggcagtgccgggacaaagaatgtaccgagtgcgaccctctgcctaatcctagcctgaccgccagaagcagccaggctttgtctcctcatcctcagcctacacatctgccctacgtgtccgagatgctggaagccagaacagccggccatatgcagaccctggccgactttagacagctgcccgccagaacactgagcacccattggcctccacagagaagcctgtgcagcagcgacttcatccggatcctcgtgatcttcagcggcatgttcctggtgttcacactggctggcgccctgtttctgcataagcgcggtcggaagaagctgctgtacatctttaagcaacccttcatgaggcctgtgcagactactcaagaggaggacggctgttcatgccggttcccagaggaggaggaaggcggctgcgaactgcgcgtgaaattcagccgcagcgcagatgctccagcctacaagcaggggcagaaccagctctacaacgaactcaatcttggtcggagagaggagtacgacgtgctggacaagcggagaggacgggacccagaaatgggcgggaagccgcgcagaaagaatccccaagagggcctgtacaacgagctccaaaaggataagatggcagaagcctatagcgagattggtatgaaaggggaacgcagaagaggcaaaggccacgacggactgtaccagggactcagcaccgccaccaaggacacctatgacgctcttcacatgcaggccctgccgcctcgggaattcggatctggcgccaccaatttcagcctgctgaaacaggctggcgacgtggaagagaaccctggacctatgagaatcagcaagccccacctgagatccatcagcatccagtgctacctgtgcctgctgctgaacagccactttctgacagaggccggcatccacgtgttcatcctgggctgtttttctgccggcctgcctaagaccgaggccaactgggttaacgtgatcagcgacctgaagaagatcgaggacctgatccagagcatgcacatcgacgccacactgtacaccgagagcgacgtgcaccctagctgtaaagtgaccgccatgaagtgctttctgctggaactgcaagtgatcagcctggaaagcggcgacgccagcatccacgacaccgtggaaaacctgatcatcctggccaacaacagcctgagcagcaacggcaatgtgaccgagtccggctgcaaagagtgcgaggaactggaagagaagaatatcaaagagttcctgcagagcttcgtgcacatcgtgcagatgttcatcaacaccagcgaattctaactcgag

SEQ ID NO: 32 - protein, 554 aa, Artificial Sequence
  CAR-P2A-IL21 polypeptide
MARPHPWWLCVLGTLVGLSATPAPKSCPERHYWAQGKLCCQMCEPGTFLVKDCDQHRKAAQCDPCIPGVSFSPDHHTRPHCESCRHCNSGLLVRNCTITANAECACRNGWQCRDKECTECDPLPNPSLTARSSQALSPHPQPTHLPYVSEMLEARTAGHMQTLADFRQLPARTLSTHWPPQRSLCSSDFIRILVIFSGMFLVFTLAGALFLHKRGRKKLLYIFKQPFMRPVQTTQEEDGCSCRFPEEEEGGCELRVKFSRSADAPAYKQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPREFGSGATNFSLLKQAGDVEENPGPMRSSPGNMERIVICLMVIFLGTLVHKSSSQGQDRHMIRMRQLIDIVDQLKNYVNDLVPEFLPAPEDVETNCEWSAFSCFQKAQLKSANTGNNERIINVSIKKLKRKPPSTNAGRRQKHRLTCPSCDSYEKKPPKEFLERFKSLLQKMIHQHLSSRTHGSEDSEF

SEQ ID NO: 33 - DNA, 1683 bp, Artificial Sequence
  CAR-P2A-IL21 construct
actagtgccaccatggccagacctcatccttggtggctgtgtgtgctgggcacactcgttggcctgtctgctacaccagctcctaagagctgccccgagagacactattgggcccagggcaagctgtgctgccagatgtgtgaacctggcaccttcctggtcaaggactgcgaccagcacagaaaggccgctcagtgcgatccttgtatccccggcgtgtccttctctcccgaccaccacacaagacctcactgcgagagctgcagacactgcaattctggactgctcgtgcggaactgcaccatcacagccaatgccgagtgcgcctgcagaaatggctggcagtgccgggacaaagaatgtaccgagtgcgaccctctgcctaatcctagcctgaccgccagaagcagccaggctttgtctcctcatcctcagcctacacatctgccctacgtgtccgagatgctggaagccagaacagccggccatatgcagaccctggccgactttagacagctgcccgccagaacactgagcacccattggcctccacagagaagcctgtgcagcagcgacttcatccggatcctcgtgatcttcagcggcatgttcctggtgttcacactggctggcgccctgtttctgcataagcgcggtcggaagaagctgctgtacatctttaagcaacccttcatgaggcctgtgcagactactcaagaggaggacggctgttcatgccggttcccagaggaggaggaaggcggctgcgaactgcgcgtgaaattcagccgcagcgcagatgctccagcctacaagcaggggcagaaccagctctacaacgaactcaatcttggtcggagagaggagtacgacgtgctggacaagcggagaggacgggacccagaaatgggcgggaagccgcgcagaaagaatccccaagagggcctgtacaacgagctccaaaaggataagatggcagaagcctatagcgagattggtatgaaaggggaacgcagaagaggcaaaggccacgacggactgtaccagggactcagcaccgccaccaaggacacctatgacgctcttcacatgcaggccctgccgcctcgggaattcggatctggcgccaccaatttcagcctgctgaaacaggctggcgacgtggaagagaaccctggacctatgagatccagtcctggcaacatggagaggattgtcatctgtctgatggtcatcttcttggggacactggtccacaaatcaagctcccaaggtcaagatcgccacatgattagaatgcgtcaacttatagatattgttgatcagctgaaaaattatgtgaatgacttggtccctgaatttctgccagctccagaagatgtagagacaaactgtgagtggtcagctttttcctgctttcagaaggcccaactaaagtcagcaaatacaggaaacaatgaaaggataatcaatgtatcaattaaaaagctgaagaggaaaccaccttccacaaatgcagggagaagacagaaacacagactaacatgcccttcatgtgattcttatgagaaaaaaccacccaaagaattcctagaaagattcaaatcacttctccaaaagatgattcatcagcatctgtcctctagaacacacggaagtgaagattccgaattctaactcgag

SEQ ID NO: 34 - protein, 961 aa, Artificial Sequence
  CAR-P2A-IL12 polypeptide
MARPHPWWLCVLGTLVGLSATPAPKSCPERHYWAQGKLCCQMCEPGTFLVKDCDQHRKAAQCDPCIPGVSFSPDHHTRPHCESCRHCNSGLLVRNCTITANAECACRNGWQCRDKECTECDPLPNPSLTARSSQALSPHPQPTHLPYVSEMLEARTAGHMQTLADFRQLPARTLSTHWPPQRSLCSSDFIRILVIFSGMFLVFTLAGALFLHKRGRKKLLYIFKQPFMRPVQTTQEEDGCSCRFPEEEEGGCELRVKFSRSADAPAYKQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPREFGSGATNFSLLKQAGDVEENPGPMCPARSLLLVATLVLLDHLSLARNLPVATPDPGMFPCLHHSQNLLRAVSNMLQKARQTLEFYPCTSEEIDHEDITKDKTSTVEACLPLELTKNESCLNSRETSFITNGSCLASRKTSFMMALCLSSIYEDLKMYQVEFKTMNAKLLMDPKRQIFLDQNMLAVIDELMQALNFNSETVPQKSSLEEPDFYKTKIKLCILLHAFRIRAVTIDRVMSYLNASGSGATNFSLLKQAGDVEENPGPMCHQQLVISWFSLVFLASPLVAIWELKKDVYVVELDWYPDAPGEMVVLTCDTPEEDGITWTLDQSSEVLGSGKTLTIQVKEFGDAGQYTCHKGGEVLSHSLLLLHKKEDGIWSTDILKDQKEPKNKTFLRCEAKNYSGRFTCWWLTTISTDLTFSVKSSRGSSDPQGVTCGAATLSAERVRGDNKEYEYSVECQEDSACPAAEESLPIEVMVDAVHKLKYENYTSSFFIRDIIKPDPPKNLQLKPLKNSRQVEVSWEYPDTWSTPHSYFSLTFCVQVQGKSKREKKDRVFTDKTSATVICRKNASISVRAQDRYYSSSWSEWASVPCSEF

SEQ ID NO: 35 - DNA, 2904 bp, Artificial Sequence
  CAR-P2A-IL12 construct
actagtgccaccatggccagacctcatccttggtggctgtgtgtgctgggcacactcgttggcctgtctgctacaccagctcctaagagctgccccgagagacactattgggcccagggcaagctgtgctgccagatgtgtgaacctggcaccttcctggtcaaggactgcgaccagcacagaaaggccgctcagtgcgatccttgtatccccggcgtgtccttctctcccgaccaccacacaagacctcactgcgagagctgcagacactgcaattctggactgctcgtgcggaactgcaccatcacagccaatgccgagtgcgcctgcagaaatggctggcagtgccgggacaaagaatgtaccgagtgcgaccctctgcctaatcctagcctgaccgccagaagcagccaggctttgtctcctcatcctcagcctacacatctgccctacgtgtccgagatgctggaagccagaacagccggccatatgcagaccctggccgactttagacagctgcccgccagaacactgagcacccattggcctccacagagaagcctgtgcagcagcgacttcatccggatcctcgtgatcttcagcggcatgttcctggtgttcacactggctggcgccctgtttctgcataagcgcggtcggaagaagctgctgtacatctttaagcaacccttcatgaggcctgtgcagactactcaagaggaggacggctgttcatgccggttcccagaggaggaggaaggcggctgcgaactgcgcgtgaaattcagccgcagcgcagatgctccagcctacaagcaggggcagaaccagctctacaacgaactcaatcttggtcggagagaggagtacgacgtgctggacaagcggagaggacgggacccagaaatgggcgggaagccgcgcagaaagaatccccaagagggcctgtacaacgagctccaaaaggataagatggcagaagcctatagcgagattggtatgaaaggggaacgcagaagaggcaaaggccacgacggactgtaccagggactcagcaccgccaccaaggacacctatgacgctcttcacatgcaggccctgccgcctcgggaattcggatctggcgccaccaatttcagcctgctgaaacaggctggcgacgtggaagagaaccctggacctatgtgtccagcgcgcagcctcctccttgtggctaccctggtcctcctggaccacctcagtttggccagaaacctccccgtggccactccagacccaggaatgttcccatgccttcaccactcccaaaacctgctgagggccgtcagcaacatgctccagaaggccagacaaactctagaattttacccttgcacttctgaagagattgatcatgaagatatcacaaaagataaaaccagcacagtggaggcctgtttaccattggaattaaccaagaatgagagttgcctaaattccagagagacctctttcataactaatgggagttgcctggcctccagaaagacctcttttatgatggccctgtgccttagtagtatttatgaagacttgaagatgtaccaggtggagttcaagaccatgaatgcaaagcttctgatggatcctaagaggcagatctttctagatcaaaacatgctggcagttattgatgagctgatgcaggccctgaatttcaacagtgagactgtgccacaaaaatcctcccttgaagaaccggatttttataaaactaaaatcaagctctgcatacttcttcatgctttcagaattcgggcagtgactattgatagagtgatgagctatctgaatgcttccggatctggcgccaccaatttcagcctgctgaaacaggctggcgacgtggaagagaaccctggacctatgtgtcaccagcagttggtcatctcttggttttccctggtttttctggcatctcccctcgtggccatatgggaactgaagaaagatgtttatgtcgtagaattggattggtatccggatgcccctggagaaatggtggtcctcacctgtgacacccctgaagaagatggtatcacctggaccttggaccagagcagtgaggtcttaggctctggcaaaaccctgaccatccaagtcaaagagtttggagatgctggccagtacacctgtcacaaaggaggcgaggttctaagccattcgctcctgctgcttcacaaaaaggaagatggaatttggtccactgatattttaaaggaccagaaagaacccaaaaataagacctttctaagatgcgaggccaagaattattctggacgtttcacctgctggtggctgacgacaatcagtactgatttgacattcagtgtcaaaagcagcagaggctcttctgacccccaaggggtgacgtgcggagctgctacactctctgcagagagagtcagaggggacaacaaggagtatgagtactcagtggagtgccaggaggacagtgcctgcccagctgctgaggagagtctgcccattgaggtcatggtggatgccgttcacaagctcaagtatgaaaactacaccagcagcttcttcatcagggacatcatcaaacctgacccacccaagaacttgcagctgaagccattaaagaattctcggcaggtggaggtcagctgggagtaccctgacacctggagtactccacattcctacttctccctgacattctgcgttcaggtccagggcaagagcaagagagaaaagaaagatagagtcttcacggacaagacctcagccacggtcatctgccgcaaaaatgccagcattagcgtgcgggcccaggaccgctactatagctcatcttggagcgaatgggcatctgtgccctgcagtgaattctaactcgag